(19) United States
(12) Patent Application Publication
(10) Pub. No.: US 2016/0213777 A1
(43) Pub. Date: Jul. 28, 2016

(54) Novel Delivery Compositions and Methods of Using Same

(71) Applicant: THOMAS JEFFERSON UNIVERSITY, Philadelphia, PA (US)

(72) Inventors: Sunday A. SHOYELE, Cherry Hill, NJ (US); Asha R. SRINIVASAN, Mysore, Karnataka State (IN)

(21) Appl. No.: 14/917,491

(22) PCT Filed: Sep. 12, 2014
(86) PCT No.: PCT/US14/55438

§ 371 Date: Mar. 8, 2016

Related U.S. Application Data
(60) Provisional application No. 61/876,969, filed on Sep. 12, 2013.

(51) Int. Cl. A61K 39/395; A61K 31/713; C07K 16/18; C12N 15/113; A61K 9/16; C07K 16/22

(52) U.S. Cl. CPC A61K 39/3955; A61K 9/1641; C07K 16/22; C07K 16/18; C12N 15/113; A61K 31/713; C07K 2317/77; C12N 2310/14; C12N 2320/32; C07K 2317/94; C07K 2317/92

(57) ABSTRACT

This invention provides compositions comprising at least one protein nanoparticle comprising a protein and a non-ionic surfactant. In certain embodiments, the nanoparticle further comprises a therapeutic agent, such as a siRNA. In other embodiments, the nanoparticle further comprises a cell surface receptor ligand. Also included in the invention are methods of preparing the compositions of the present invention, and methods of treating, ameliorating or preventing a disease or disorder in a subject in need thereof using the compositions of the present invention.

### CROSS-REFERENCE TO RELATED APPLICATIONS

The present application claims priority under 35 U.S.C. §119(e) to U.S. Provisional Patent Application No. 61/876,969, filed Sep. 12, 2013, which is incorporated herein by reference in its entirety.

### BACKGROUND OF INVENTION

There is great interest in developing compositions that allow for the delivery of biologically active agents to a cell. Such compositions may be used in therapeutic treatments against chronic or acute diseases or disorders in a subject in need thereof.

Proteins are chief players in cellular metabolism, being regularly used as therapeutic agents in medicine. Native or recombinant proteins, along with monoclonal antibodies (mAbs), are examples of proteins that find use in medicinal applications. However, proteins in general are poorly absorbed across biological membranes, and are thus generally delivered intravenously (Kegan et al., 2011, Pharm Res. DOI 10.1007/s11095-011-0578-3; Roskos et al., 2004, Drug Develop Res. 61:108-120). The parenteral administration route has several disadvantages, including patient discomfort, potential high cost and the risk of needle-stick injuries (Patton, 1997, Chemtech. 27:34-38; Shoyele & Cawthorne, 2006, Adv. Drug Deliv. Rev. 58:1009-1029; Shoyele & Slowey, 2006, Intl. J. Pharm. 314:1-8). The pulmonary administration route offers an excellent alternative for proteins (especially antibodies) that are targeted towards local lung diseases. Monoclonal antibodies such as bevacizumab, anatumomab, benralizumab, enokizumab, mitumomab, oxelumab and palivizumab have gained FDA approval for the treatment of lung diseases, such as asthma, lung cancers and respiratory syncytial virus infection.

Microparticles (rather than nanoparticles) are the particles of choice for delivering drugs, including proteins, to the lung by inhalation, due to the widespread belief that nanoparticles are in a size range not suitable for deep lung delivery (Azarmi et al., 2008, Adv. Drug Deliv. Rev. 60:863-875; Sham et al., 2004, Intl. J. Pharm. 269:457-467). On the other hand, nanoparticles in pulmonary drug delivery may offer advantages such as: (1) the potential to achieve relatively uniform distribution of drug dose among the alveoli; (2) an achievement of enhanced solubility of the drug than its aqueous solubility; (3) decreased incidence of side effects; (4) improved patience compliance; and (5) the potential of drug internalization by cells (Mansour et al., 2009, Intl. J. Nanomed. 4:299-319; Sung et al., 2007, Trends Biotechnol. 25:563-570; Bailey& Berkland, 2009, Med. Res. Rev. 29:196-212).

Monoclonal antibodies (mAbs) currently do not benefit fully from the unique advantages offered by nanosystems in pulmonary drug delivery, mainly because of their labile molecular structure. The higher order structures of proteins, i.e. secondary, tertiary and sometimes quaternary structures, are stabilized by weak physical interactions such as hydrogen bonding, electrostatic attraction, van der Waal force and hydrophobic interaction, rather than the stronger covalent bond (Shoyele & Slowey, 2006, Intl. J. Pharm. 314:1-8). Antibodies are thus susceptible to various stresses involved in nanoparticle fabrication.

Bevacizumab, a humanized mAb against vascular endothelial growth factor (VEGF), has shown encouraging signs in the treatment of non-small cell lung cancer (NSCLC) when used alone or in combination with chemotherapy. The FDA approved it in 2006 for use along with paclitaxel and carboplatin as first-line treatment for those with advanced NSCLC. Cancer cells tend to overexpress VEGF, a potent stimulator of angiogenesis, facilitating cancer growth and metastasis. Internalization of bevacizumab into cancer cells is highly important as intracellular pool of VEGF could be responsible for resistance to bevacizumab in cancer therapy (Inoue et al., 2007, Mol. Ther. 15:287-294; Hamerlik et al., 2012, J. Exp. Med. 209:507-520). To this end, intracellular VEGF provides a compelling target for mAbs in cancer therapy.

RNA interference (RNAi) is a very effective tool in the knockdown of specific oncogenes in cancer cells. siRNA is the most widely studied form of RNAi, and has a promising therapeutic potential in cancer and other diseases such as autoimmune diseases and infectious diseases (Jagani et al., 2011, Arzneimittelforscchung. 61:577-586). Nevertheless, challenges still occur in the development of siRNA as a therapeutic agent due to siRNA's susceptibility to enzymatic degradation in blood, non-specific uptake by cells, and the difficulty involved in its transfection into cells due to its relatively large size and polarity (Kim, 2003, J. Korean Med. Sci. 18:309; Shim & Kwon, 2010, FEBS. J. 277:4814-4827). Clearance by the reticulo-endothelial system (RES) is another limiting factor affecting the possible therapeutic application of siRNA (de Fougerolles et al., 2006, Nat. Rev. Drug Discov. 6:443-453; Kalluri et al., 2009, Angew. Chem. Int. Engl. 48:9668-9671; Xue & Wong, 2011, ACS Nano 5:367-373).

To achieve an efficient knockdown by siRNA, various types of delivery systems have been investigated. While viral vectors have been known to transduce cells efficiently, their use in translational medicine is hampered by the possibility of viral toxicity and immunogenic and inflammatory reactions. Non-viral vectors such as lipid-based nanoparticles and mesoporous silica are being investigated as possible delivery systems for efficient siRNA transfection. In order to achieve an efficient delivery of siRNA, the delivery system must have the following properties: protect siRNA from nuclease degradation during transportation in systemic circulation; have minimal RES uptake, thereby allowing for long blood circulation time; allow for effective endosomal escape following internalization by host cells; and most important, must not elicit immunological and inflammatory reaction. Lipid nanoparticles demonstrate major limitations: siRNA delivery by lipid-based nanoparticles is substantially reduced, because approximately 70% of the internalized siRNA undergoes exocytosis through egress of the lipid nanoparticles from late endosomes and lysosomes. Use of poly (D,L)-lactide-co-glycolide (PLGA) nanoparticles to deliver siRNA is also problematic, because this polymer is negatively charged and interacts minimally with negatively charged siRNA, thus reducing cellular internalization. There is thus a growing need for a smart nanoparticle delivery system for efficient and stable siRNA transfection.

Presently, nanoparticulate systems used in drug delivery include: polymer-based drug carriers (including polymeric nanospheres, polymeric micelles and dendrimers), liposomes, viral nanoparticles, and carbon tubes (Cho et al., 2008, Clin. Cancer Res. 14:1310-1316). The processes involved in the fabrication of these nanoparticles often lead to degradation and sometimes, loss of biological activity in the biological agent (Peterson et al., 2010, Acta Biomater. 6:3873-3881; Kim et al., 2011, Biochem. Biophys. Res. Comm. 408:225-229; Son et al., 2009, Intl. J. Pharm. 368:178-185; Bailey et al., 2008, Langmuir. 24(23):13614-13620). Further, some materials used to formulate these nanoparticles may have toxic effects and may not be viable in therapeutic treatments (Zhang et al., 2008, Bioconj. Chem. 19:1880-1887; Kleemann et al., 2005, J. Cont. Rel. 109:299-316).

There is a need in the art for novel and versatile delivery compositions, which are compatible with biological systems and therapeutic agents. There is a further need for novel compositions and formulations that allow for pulmonary delivery of therapeutically useful proteins, such as native or recombinant proteins or monoclonal antibodies. There is a further need for novel compositions and formulations that allow for efficient and stable siRNA transfection. The present invention satisfies these needs.

### BRIEF SUMMARY OF THE INVENTION

In one aspect, the invention provides a composition comprising at least one protein nanoparticle. In another aspect, the invention provides a method of preparing at least one protein nanoparticle. In yet another aspect, the invention provides a method of treating, ameliorating or preventing a disease or disorder in a subject in need thereof, the method comprising administering to the subject a pharmaceutically effective amount of a composition comprising at least one protein nanoparticle comprising a protein and a non-ionic surfactant. In yet another aspect, the invention provides a kit comprising a composition comprising at least one protein nanoparticle. In yet another aspect, the invention provides a kit comprising a non-ionic surfactant and optionally a protein.

In certain embodiments, the compositions of the present invention have low or minimal uptake by phagocytic cells of the reticulo-endothelial system (RES) in a subject to which the compositions are administered. In other embodiments, the compositions of the present invention cause low or minimal immunostimulation in a subject to which the compositions are administered. In yet other embodiments, the compositions of the present invention have increased in vivo circulation time as compared to the “unformulated” protein and/or therapeutic agent that comprise(s) the compositions (i.e., the protein and/or therapeutic agent that is/are not within the nanoparticles of the present invention). In yet other embodiments, the compositions of the present invention protect the therapeutic agent incorporated therein from nuclease activity upon administration to a subject.

In certain embodiments, the protein nanoparticle is prepared by a method comprising adjusting the pH of a solution comprising a protein and a non-ionic surfactant to about the isoelectric point of the protein, thereby forming a precipitate comprising the protein nanoparticle, wherein the protein nanoparticle comprises at least a fraction of the protein and at least a fraction of the non-ionic surfactant.

In certain embodiments, the protein comprises at least one selected from the group consisting of a hormone, immunomodulator, cytokine, interferon, interleukin, and enzyme. In other embodiments, the protein comprises an antibody. In yet other embodiments, the antibody comprises IgG. In yet other embodiments, the IgG is human. In yet other embodiments, the antibody comprises a monoclonal antibody. In yet other embodiments, the monoclonal antibody comprises at least one selected from the group consisting of bevacizumab, anatumomab, benralizumab, enokizumab, mitumomab, oxelumab, and palivizumab.

In certain embodiments, the solution further comprises at least one therapeutic agent, and the protein nanoparticle comprises at least a fraction of the at least one therapeutic agent. In other embodiments, the at least therapeutic agent is selected from the group consisting of an organic compound, inorganic compound, pharmacological drug, radiopharmaceutical, protein, peptide, polysaccharide, nucleic acid, siRNA, RNAi, short hairpin RNA, antisense nucleic acid, ribozyme and dominant negative mutant. In yet other embodiments, the at least one therapeutic agent comprises a siRNA. In yet other embodiments, the protein comprises IgG and the nanoparticle further comprises a therapeutic agent comprising a siRNA.

In certain embodiments, the solution further comprises at least one cell surface receptor ligand, and the protein nanoparticle comprises at least a fraction of the at least one cell surface receptor ligand. In other embodiments, the at least one ligand binds to at least one selected from the group consisting of neurotensin receptor-1, human epidermal growth factor receptor-2 (HER-2), folate receptor, insulin-like growth receptor (IGF), and epidermal growth factor receptor (EGFR).

In certain embodiments, the non-ionic surfactant comprises at least one selected from the group consisting of an alkyl polyethylene oxide, alkylphenol polyethylene oxide, copolymer of polyethylene oxide and polypropylene oxide, alkyl polyglucoside, fatty alcohol, cocamide MEA, cocamide DEA, and polysorbate. In other embodiments, the alkyl polyethylene oxide comprises at least one selected from the group consisting of a diethylene glycol hexadecyl ether, polyethylene glycol oleyl ether, diethylene glycol octadecyl ether, polyoxyethylene stearyl ether, polyethylene glycol hexadecyl (cetyl) ether, polyethylene glycol dodecyl (lauryl) ether, decaethylene glycol oleyl ether, polyethylene glycol octadecyl ether, and polyethylene glycol octadecyl ether. In yet other embodiments, the non-ionic surfactant comprises a copolymer of polyethylene oxide and polypropylene oxide.

In certain embodiments, the average diameter of the at least one protein nanoparticle ranges from about 1 nm to about 1,000 nm. In other embodiments, the average diameter of the at least one protein nanoparticle ranges from about 100 nm to about 900 nm. In other embodiments, the average diameter of the at least one protein nanoparticle ranges from about 100 nm to about 700 nm.

In certain embodiments, the concentration of the non-ionic surfactant in the solution ranges from about 5% to about 20,000% of the CMC of the surfactant. In other embodiments, the concentration of the non-ionic surfactant in the solution ranges from about 100% to about 20,000% of the CMC of the surfactant. In yet other embodiments, the concentration of the non-ionic surfactant in the solution ranges from about 300% to about 10,000% of the CMC of the surfactant. In yet other embodiments, the concentration of the non-ionic surfactant in the solution ranges from about 300% to about 5,000% of the CMC of the surfactant.

In certain embodiments, the composition of the present invention further comprises a pharmaceutically acceptable carrier.

In certain embodiments, the method of the present invention comprises adjusting the pH of a solution comprising a protein and a non-ionic surfactant to about the isoelectric point of the protein, thereby forming a precipitate comprising the at least one protein nanoparticle; wherein the at least one protein nanoparticle comprises at least a fraction of the protein and at least a fraction of the non-ionic surfactant.

In certain embodiments, the precipitate is further purified to remove protein or non-ionic surfactant that is not associated with the at least one protein nanoparticle, thereby generating a composition comprising the at least one protein nanoparticle. In other embodiments, the composition comprising at least one protein nanoparticle is further lyophilized.

In certain embodiments, wherein the at least one protein nanoparticle is precipitated from a solution comprising the protein and the non-ionic surfactant, further wherein the pH of the solution is equal to about the isoelectric point of the protein, and further wherein the concentration of the non-ionic surfactant in the solution ranges from about 5% to about 20,000% of the CMC of the surfactant.

In certain embodiments, the composition is administered to the subject by an intrapulmonary, intrabronchial, inhalational, intranasal, intratracheal, intravenous, intramuscular, subcutaneous, topical, transdermal, oral, buccal, rectal, pleural, peritoneal, vaginal, epidural, otic, intraocular, or intrathecal route. In other embodiments, the composition is administered to the subject by an intrapulmonary, intrabronchial, inhalational, intranasal, intratracheal, intravenous, intramuscular, subcutaneous or topical route.

In certain embodiments, the disease or disorder is selected from the group consisting of colon cancer, rectum cancer, lung cancer, glioblastoma, renal cell cancer, non-small cell lung cancer, small cell lung cancer, asthma, respiratory syncytial virus (RSV) infection, and any combinations thereof. In other embodiments, the disease or disorder comprises a cancer comprising a KRAS mutation.

In certain embodiments, the kit further comprises an applicator. In other embodiments, the kit further comprises an instructional material for the use of the kit. In yet other embodiments, the instruction material comprises instructions for treating, ameliorating or preventing a disease or disorder in a subject in need thereof. In yet other embodiments, the instruction material comprises instructions for preparing a protein nanoparticle comprising at least a fraction of the non-ionic surfactant and at least a fraction of the protein.

In certain embodiments, the subject is a mammal. In other embodiments, the mammal is human.

### BRIEF DESCRIPTION OF THE DRAWINGS

For the purpose of illustrating the invention, there are depicted in the drawings certain embodiments of the present invention. However, the invention is not limited to the precise arrangements and instrumentalities of the embodiments depicted in the drawings.

FIG. 1 illustrates a SEM micrograph of the unprocessed IgG particles. Scale bar=20 μm.

FIG. 2 illustrates a SEM micrograph of IgG particles precipitated from 5 mg/ml solution in the absence of any surfactant. Scale bar=1 μm.

FIG. 3 illustrates a SEM micrograph of IgG particles precipitated from 5 mg/ml solution in the presence of 0.1% w/v TWEEN® 80. Scale bar=200 nm.

FIG. 4 illustrates a SEM micrograph of IgG particles precipitated from 5 mg/ml solution in the presence of 0.1% w/v TWEEN® 20. Scale bar=200 nm.

FIG. 5 illustrates a SEM micrograph of IgG particles precipitated from 5 mg/ml solution in the presence of 0.1% w/v BRIJ® 97. Scale bar=200 nm.

FIG. 6 is a bar graph illustrating % specific activity of IgG nanoparticles prepared from TWEEN® 80-containing solutions, as determined by ELISA as fraction of the starting activity prior to processing.

FIG. 7 is a bar graph illustrating % specific activity of IgG nanoparticles prepared from TWEEN® 20-containing solutions, as determined by ELISA as fraction of the starting activity prior to processing.

FIG. 8 is a bar graph illustrating % specific activity of IgG nanoparticles prepared from BRIJ® 97-containing solutions, as determined by ELISA as fraction of the starting activity prior to processing.

FIG. 9 illustrates a SE-HPLC chromatogram of the unprocessed IgG solution (positive control).

FIG. 10 illustrates far UV CD spectra from nanoparticles generated from 7.5 mg/ml IgG solutions in the presence of TWEEN® 80.

FIG. 11, comprising FIGS. 11A-11E, illustrates SEM micrographs of bevacizumab particles. FIG. 11A: Lyophilized bevacizumab from the supplied solution after dialysis to remove any excipients in the solution (scale bar, 20 μm). FIG. 11B: surfactant-free nanoparticles (scale bar, 200 nm). FIG. 11C: 0.1% TWEEN® 80 bevacizumab nanoparticle (scale bar, 200 nm). FIG. 11D: 0.1% TWEEN® 20 bevacizumab nanoparticles (scale bar, 200 nm). FIG. 11E: 0.1% BRIJ® 97 bevacizumab nanoparticles (scale bar, 200 nm).

FIG. 12 is a bar graph illustrating the measurement of HUVEC proliferation effect of various concentrations of VEGF.

FIG. 13 is a set of bar graphs illustrating metabolic activity and cellular proliferation (as measured by fluorescence of Alamar blue dye) of HUVEC cells in the presence of VEGF, which had been pre-incubated with various concentrations of TWEEN® 80-bevacizumab compositions.

FIG. 14 is a bar graph illustrating metabolic activity and cellular proliferation (as measured by fluorescence of Alamar blue dye) of HUVEC cells in the presence of VEGF, which had been pre-incubated with various concentrations of TWEEN® 20-bevacizumab compositions.

FIG. 15 is a bar graph illustrating metabolic activity and cellular proliferation (as measured by fluorescence of Alamar blue dye) of HUVEC cells in the presence of VEGF, which had been pre-incubated with various concentrations of BRIJ® 97-bevacizumab compositions.

FIG. 16 is a bar graph illustrating the cytotoxicity of reconstituted TWEEN® 80-bevacizumab compositions against A549 cell lines, using the MTT assay.

FIG. 17 is a bar graph illustrating the cytotoxicity of reconstituted TWEEN® 20-bevacizumab compositions against A549 cell lines, using the MTT assay.

FIG. 18 is a bar graph illustrating the cytotoxicity of reconstituted BRIJ® 97-bevacizumab compositions against A549 cell lines, using the MTT assay.

FIG. 19 illustrates CD spectra corresponding to secondary structure studies of selected compositions.

FIG. 20 illustrates the design and function of an Anderson cascade impactor.

FIG. 21, comprising FIGS. 21A-21D, is a series of photographs illustrating cells incubated with bevacizumab nanoparticles.

FIG. 22, comprising FIGS. 22A-22D, is a series of photographs illustrating cells incubated with unprocessed bevacizumab nanoparticles.

FIG. 23, comprising FIGS. 23A-23B, is a series of photographs illustrating the fluorescence imaging of the bevacizumab nanoparticles (FIG. 23A) and unprocessed bevacizumab (FIG. 23B) after labeling with FITC. Scale bar=5 μm.

FIG. 24, comprising FIGS. 24A-24D, is a series of photographs illustrating cells incubated with FITC-labeled bevacizumab nanoparticles.

FIG. 25, comprising FIGS. 25A-25D, is a series of fluorescence micrographs of A549 cells after 60 minute incubation at 37° C. with FITC-bevacizumab nanoparticles (illustrating a single cell from FIG. 24). The nucleus was stained with DAPI while the cell membrane was stained with wheat germ agglutinin labeled with Alexa-Fluor-555.

FIG. 26, comprising FIGS. 26A-26D, is a series of photographs illustrating cells incubated with FITC-labeled unprocessed bevacizumab particles. No particles were observed in the cells.

FIG. 27, comprising FIGS. 27A-27D, is a series of photographs illustrating A549 cells incubated with 0.1% TWEEN® in PBS as a control. The cells showed no green staining, confirming that the FITC-labeled particles were responsible for the green stain observed in FIGS. 24-25.

FIG. 28, comprising FIGS. 28A-28D, illustrates MRC-5 cells incubated with labeled bevacizumab nanoparticles after 60 minutes. The nucleus was stained with DAPI while the cell membrane was stained with wheat germ agglutinin labeled with Alexa-Fluor-555 (WGA). Very few nanoparticles could be seen in the cells, indicating that the bevacizumab nanoparticles did not accumulate in the MRC-5 cells. This suggested selectivity between cancer cell and normal cells by the negatively charged nanoparticles.

FIG. 29, comprising FIGS. 29A-29B, is a series of TEM micrographs of A549 cells following incubation with bevacizumab nanoparticles for 15 minutes. FIG. 29A depicts clathrin pits around the nanoparticles (arrow) as they were being internalized. FIG. 29B depicts the nanoparticles in a vesicle (early endosome) in the cell.

FIG. 30, comprising FIGS. 30A-30B, is a series of TEM micrographs of A549 cells following incubation with bevacizumab nanoparticles for 60 minutes. FIG. 30A depicts the cell internalizing a particle by macro-pinocytosis (arrow). FIG. 30B depicts the particles in the vesicle (arrow) gradually undergoing dissolution.

FIG. 31 is a bar graph illustrating the finding that internalization of bevacizumab nanoparticles by A549 cells increased as incubation time increased from 15 minutes to 60 minutes.

FIG. 32 is a bar graph illustrating higher accumulation of nanoparticles in A549 in comparison to MRC-5 cell line, as indicated by the finding that the fluorescence intensity in A549 was 3 times higher when compared to MRC. Unprocessed bevacizumab particles were not internalized by either A549 or MRC-5 cells. Cells were incubated for 60 minutes with particles in all cases.

FIG. 33 is a graph illustrating the overlay of the chromatograms obtained from SE-HPLC analysis of distinct bevacizumab nanoparticles following reconstitution in 0.1 M acetate buffer (pH 5). The main peaks at approximately 11 min suggest the presence of dimers in the reconstituted unprocessed bevacizumab, while the peaks present at approximately 14 min and 15 min indicate the presence of fragments.

FIG. 34 is a bar graph illustrating the experimental results relating to probing the mechanisms of cellular internalization using chemical inhibitors. % internalization was normalized to particle internalization in the absence of inhibitors.

FIG. 35 is a reproduction of a confocal micrograph of A549 cells contacted with anti-survivin nanoparticles of the present invention.

FIG. 36 is a bar graph illustrating the dose-dependent anti-proliferative effect of anti-survivin nanoparticles against A549 cells in vitro.

FIG. 37 is a graph illustrating the in vivo anti-tumor effect of injected anti-survivin nanoparticles in the mouse. Changes in tumor volume were recorded following treatment with anti-survivin mAb nanoparticles. Tumor volume was measured using external calipers after treatments on days 3 and 7 after the establishment of lung tumor.

FIG. 38 is a non-limiting schematic of a siRNA-loaded mAb nanoparticle.

FIG. 39 is a bar graph illustrating the uptake of self-associated mAb nanoparticles by A549 cells, as compared to the uptake of chitosan nanoparticles. A549 cells were incubated with the same concentrations (100 μg/ml) of nanoparticles before flow cytometry analysis.

FIG. 40, comprising FIGS. 40A-40D, illustrate TEM micrographs of (FIG. 40A) nanoparticles at lower magnification, (FIG. 40B) IgG-nanoparticle at higher magnification, (FIG. 40C) siRNA-IP nanoparticle and (FIG. 40D) IP-nanoparticle.

FIG. 41 comprising a series of images illustrating serum stability of siRNA in 50% FBS over a period of time.

FIG. 42 is a graph illustrating in vitro siRNA release profile from siRNA-encapsulated nanoparticles at pH 5.

FIG. 43 is a graph illustrating in vitro siRNA release profile from siRNA-encapsulated nanoparticles at pH 7.2.

FIG. 44 is a set of images illustrating cellular uptake and intracellular trafficking of siGLO-IP-nanoparticles. Cell were fixed at the indicated time points and stained with DAPI (blue/light gray) and Lysotracker (red/dark grey). Images were captured using fluorescence microscopy.

FIG. 45 is an image illustrating western blot analysis of KRAS protein expression in A549 cells. Lipo represents lipofectamine transfection, IgG represents siG12S-IgG nanoparticles, POLO represents siG12S-IP nanoparticles.

FIG. 46 is a bar graph illustrating KRAS expression. KRAS Messenger RNA (mRNA) expression levels were determined in control, scramble RNA and siRNA nanoparticle treated A549 cells using TaqMan gene expression assay system. Histograms illustrate the mean±SEM of quantified mRNA expression levels normalized to GAPDH mRNA levels of each sample.

FIG. 47 is a bar graph illustrating in vitro cytotoxicity of various siRNA formulations in A549 cells. The data is presented as the mean±SD of five independent experiments.

FIG. 48 is a graph illustrating in vitro cytotoxicity of IP-nanoparticles in A549 cells. The data is presented as the mean±SD of five independent experiments.

FIG. 49 is a graph illustrating in vitro cytotoxicity of a combinations of erlotinib+siG12S (50 nM)-loaded IP nanoparticles in A549 cells. The data is presented as the mean±SD of five independent experiments.

FIG. 50, comprising FIGS. 50A-50B, is a set of bar graphs illustrating immuno-stimulatory effects of siG12S-loaded nanoparticles on 24 h murine macrophage production of (FIG. 50A) TNF-α and (FIG. 50B) IL-6.

FIG. 51 is a set of images illustrating phagocytosis of latex beads by RAW 264.7 macrophage cell monitored by fluorescence microscopy.

FIG. 52 is a set of images illustrating phagocytosis of nanoparticles by RAW 264.7 macrophage cell after 4 h monitored by fluorescence microscopy. IP-nanoparticles are in the upper panel, while IgG-nanoparticles are in the lower panel.

### DETAILED DESCRIPTION OF INVENTION

The present invention relates in part to the unexpected discovery of a novel method of producing protein nanoparticles. In one aspect, the methods of the present invention allow for careful control of the size and shape of the nanoparticles, thus enhancing their overall drug delivery properties. In certain embodiments, the nanoparticles of the present invention have improved aerosolization properties as compared to irregularly shaped (>20 μm) unprocessed particles. In other embodiments, the nanoparticles of the present invention comprise at least one therapeutic agent, wherein the at least one therapeutic agent within the nanoparticles has improved pharmacokinetics as compared to the “unformulated” therapeutic agent (i.e., the therapeutic agent that is not within the nanoparticles of the present invention). In yet other embodiments, the nanoparticles of the present invention further comprise a cell surface receptor ligand, which allows for the nanoparticles to recognize and bind to a cell that displays such cell surface receptor.

In certain embodiments, the protein comprises a native or recombinant therapeutically useful protein, such as hormones like insulin, glucagon, and somatropin; immunomodulators like cyclosporine; cytokines like interleukins, erythropoietin, and filgrastim; interferons; interleukins; enzymes like blood clotting factors, adenosine deaminase, alphal antitryptin; and peptide vaccines. In other embodiments, the protein comprises an antibody. In yet other embodiments, the protein comprises an immunoglobulin. In yet other embodiments, the immunoglobulin comprises IgA, IgD, IgE, IgG or IgM. In yet other embodiments, the antibody comprises a monoclonal antibody (mAb).

Potential applications of the mAb nanoparticles of the present invention include, in a non-limiting manner, selective targeting of intracellular oncoproteins in cancer; pulmonary delivery of mAb by dry powder inhalation; as a carrier system for delivering nucleic acids and/or small molecule to cells; and formulation of high concentration mAb dosage forms for various diseases.

In one aspect, as demonstrated herein, self-associated mAb nanoparticles are preferentially taken up by non-small lung cancer cells in comparison to normal cells due to the absence or dysfunction of tight junctions (TJ) in confluent cancer cells and increased permeability of the cancer cell membrane. Accordingly, the internalization of self-associated bevacizumab nanoparticles in NSCLC cell line (A549) was investigated in comparison to normal lung epithelial cells (MRC-5). Further, the internalization pathways of these self-associated bevacizumab nanoparticles were elucidated using transmission electron microscopy (TEM), fluorescence microscopy and flow cytometry. Retained anti-VEGF activity of the bevacizumab nanoparticles was investigated using human umbilical vein endothelial cells (HUVEC), while antiproliferative activity against NSCLC was investigated using A549 cell line. The results disclosed herein indicate that self-associated mAb nanoparticles can be selectively delivered to cancer cells.

In another aspect, the invention provides a novel hybrid nanoparticle delivery system that incorporates the benefits derived from human immunoglobulin G (human IgG) and a polyoxyethylene-polyoxypropylene block copolymer (poloxamer-188) for stable and efficient siRNA delivery. Human IgG, the main antibody isotype found in blood, is the main immunoglobulin that protects the body against infection. Without wishing to be limited by theory, as IgG is part of the body's natural defense mechanism, presence of this immunoglobulin in a nanoparticle can reduce the well documented immunogenic reaction experienced with most nanoparticle formulations. In certain embodiments, a nanoparticle delivery system comprising human IgG (or hybrid, fragment or derivative thereof) and poloxamer not only reduces immunogenic and inflammatory reactions experienced with most nanoparticles used in siRNA delivery, but also reduces macrophageal uptake by phagocytic cells, hence circumventing the RES if used therapeutically. In other embodiments, the double layer provided by these two components in a nanoparticle helps protect the loaded siRNA against endonuclease, hence allowing an efficient and stable delivery of siRNA into cell cytoplasm.

As demonstrated herein, siRNA for mutated KRAS in A549 lung carcinoma cells (siG12S) and siGLO (a fluorescent oligonucleotide that localizes to the nucleus, allowing for visual assessment of uptake into mammalian cells) were loaded into the nanoparticles of the present invention. The siRNA were well protected against serum nuclease by these hybrid nanoparticles. Expression levels of mutant KRAS^{G12S} were effectively reduced following treatment of A549 cells with siG12S-IP-nanoparticles. The treatment of A549 cells with siG12S-IP-nanoparticles led to an increase in chemosensitivity of A549 cells to erlotinib, helping to reduce resistance to EGFR-TKIs in vitro. Internalized siGLO-loaded nanoparticles escaped endocytic recycling, leading to an efficient transfection of the loaded siRNA. The nanoparticles also reduced the immunostimulatory effect of naked siRNA and prevented phagocytosis by macrophages in vitro. Thus, the novel hybrid nanoparticles can serve as an effective non-viral vector for siRNA delivery.

In one aspect, the protein nanoparticles of the present invention can be precipitated from a solution of a protein and a non-ionic surfactant (such as TWEEN® 80, TWEEN® 20 and BRIJ® 97), wherein the concentration of the non-ionic surfactant in the solution ranges from about 5% to about 20,000% of the CMC of the surfactant, when the pH of the present invention is brought to a value equal to about the isoelectric point of the protein (i.e., the pH where the protein has an overall neutral charge and minimum aqueous solubility). Without wishing to be limited by theory, the non-ionic surfactant allows for the formation of the protein nanoparticle, and protects the protein from degradation during the precipitation procedure (e.g., decomposition, aggregation or denaturation promoted by the acid or base added to the solution to bring the pH to about the isoelectric point of the protein) and any subsequent manipulation procedure (such as lyophilization or redissolution). In certain embodiments, the concentration of the surfactant in the solution is several fold higher than its critical micelle concentration. The precipitated protein nanoparticles may be separated from the supernatant by centrifugation or decantation, and further purified by rinsing with appropriate buffers. The protein nanoparticles may be resuspended in an appropriate buffer or lyophilized to yield a dry powder of protein nanoparticles.

In certain embodiments, formation of the protein nanoparticles of the present invention does not require the use of lipids or phospholipids, which are commonly used in the art to encapsulate proteins in nanospheres or liposomes. Unlike the protein nanospheres or protein liposomes known in the art, the protein nanoparticles of the present invention are readily soluble in buffers, are devoid of immunogenic and/or otherwise undesirable excipients, have high protein loading, have greater stability and shelf life, and are useful in delivering proteins to the lung of mammals via dry powder inhalation. Further, the methods of the present invention allows for the isolation of protein nanoparticles in a minimal number of steps, thus reducing the likelihood of protein degradation.

### DEFINITIONS

The definitions used in this application are for illustrative purposes and do not limit the scope used in the practice of the present invention.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, polymer chemistry, and protein chemistry are those well-known and commonly employed in the art.

As used herein, the articles “a” and “an” are used herein to refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody” as used herein refers to an immunoglobulin molecule able to specifically bind to a specific epitope on an antigen. Antibodies can be intact immunoglobulins derived from natural sources or from recombinant sources, and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antigen” or “Ag” is defined as a molecule that provokes an immune response. This immune response may involve either antibody production, or the activation of specific immunologically-competent cells, or both. The skilled artisan will understand that any macromolecule, such as virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. The present invention includes, but is not limited to, the use of partial nucleotide sequences of more than one gene, and these nucleotide sequences are arranged in various combinations to elicit the desired immune response. Moreover, a skilled artisan will understand that an antigen need not be encoded by a “gene” at all. An antigen can be generated, synthesized or derived from a biological sample. Such biological sample can include, but is not limited to, a tissue sample, tumor sample, cell or biological fluid.

As the term is used herein, “applicator” is used to identify any device including, but not limited to, a hypodermic syringe, pipette, nebulizer, vaporizer and the like, for administering the compounds and compositions used in the practice of the present invention.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopts highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

As used herein, the term “BRIJ®” is a trademark that described a non-ionic detergent comprising an oligo- or poly-ethylene glycol mono-derivatized with an aliphatic chain (an alkyl polyethylene oxide). Examples of BRIJ® compounds comprises BRIJ® 52 (polyethylene glycol hexadecyl ether; Mₙ ˜330), BRIJ® 58 (polyethylene glycol hexadecyl ether; Mₙ ˜1,124), BRIJ® 93 (polyethylene glycol oleyl ether; Mₙ ˜357), BRIJ® C10 (polyethylene glycol hexadecyl ether), BRIJ® L4 (tetraethylene glycol dodecyl ether), BRIJ® L23 (tricosethylene glycol dodecyl ether), BRIJ® O10 and BRIJ® O20 (decaethylene glycol oleyl ether), BRIJ® S2 (diethylene glycol octadecyl ether), BRIJ® S10 and BRIJ® S100 (decaethylene glycol octadecyl ether),

As used herein with respect to the compositions of the present invention, “biologically active” means that the compositions elicit a biological response in a mammal that can be monitored and characterized in comparison with an untreated mammal. In certain embodiments, the compositions are administered to the respiratory tract of the mammal. In other embodiments, the compositions are useful for inhalational, nasal, intrapulmonary, intrabronchial, or inhalation administration. In yet other embodiments, the compositions are useful for nasal, inhalational, topical, oral, buccal, rectal, pleural, peritoneal, vaginal, intramuscular, subcutaneous, transdermal, epidural, intratracheal, otic, intraocular, intrathecal or intravenous administration.

As used herein, the term “CMC” refers to critical micelle concentration. The CMC of a surfactant is defined as the solution concentration of the surfactant above which surfactant micelles form spontaneously. In certain embodiments, additional surfactant added to the system beyond the CMC value is incorporated in more micelles.

As used herein, the term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well-known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, ameliorating, or preventing shivering in a subject.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount” or “therapeutically effective amount” or “pharmaceutically effective amount” of a composition are used interchangeably to refer to the amount of the composition that is sufficient to provide a beneficial effect to the subject to which the composition is administered. The term to “treat,” as used herein, means reducing the frequency with which symptoms are experienced by a patient or subject or administering a composition to reduce the severity with which symptoms are experienced. An appropriate therapeutic amount in any individual case may be determined by one of ordinary skill in the art using routine experimentation.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

As used herein, the term “exogenous” refers to any material introduced from or produced outside an organism, cell, tissue or system.

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

“Instructional material,” as that term is used herein, includes a publication, a recording, a diagram, or any other medium of expression which can be used to communicate the usefulness of a composition of the present invention in a kit. The instructional material of the kit may, for example, be affixed to a container that contains a composition of the present invention or be shipped together with a container which contains a composition. Alternatively, the instructional material may be shipped separately from the container with the intention that the recipient uses the instructional material and a composition cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “IP-particle” or “IP-nanoparticle” refers to IgG-poloxamer-188 nanoparticle.

As used herein, the term “medical intervention” means a set of one or more medical procedures or treatments that are required for ameliorating the effects of, delaying, halting or reversing a disease or disorder of a subject. A medical intervention may involve surgical procedures or not, depending on the disease or disorder in question. A medical intervention may be wholly or partially performed by a medical specialist, or may be wholly or partially performed by the subject himself or herself, if capable, under the supervision of a medical specialist or according to literature or protocols provided by the medical specialist.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

As used herein, the terms “peptide” and “polypeptide” and “protein” are used interchangeably, and refer to a compound comprised of amino acid residues covalently linked by peptide bonds. A protein or peptide must contain at least two amino acids, and no limitation is placed on the maximum number of amino acids that can comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs and fusion proteins, among others. The polypeptides include natural peptides, recombinant peptides, synthetic peptides or a combination thereof. A peptide that is not cyclic has a N-terminus and a C-terminus. The N-terminus has an amino group, which can be free (i.e., as a NH₂ group) or appropriately protected (for example, with a BOC or a Fmoc group). The C-terminus has a carboxylic group, which can be free (i.e., as a COOH group) or appropriately protected (for example, as a benzyl or a methyl ester). A cyclic peptide does not necessarily have free N- or C-termini, since they are covalently bonded through an amide bond to form the cyclic structure.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, a “pharmaceutically acceptable carrier” means a pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “poloxamer” refers to a non-ionic triblock copolymer composed of a central hydrophobic chain of polyoxypropylene (also known as poly(propylene oxide)) flanked by two hydrophilic chains of polyoxyethylene (also known as poly(ethylene oxide)). Poloxamers are also known by the trade names SYNPERONIC®, PLURONIC®, and KOLLIPHOR®. For the generic term “poloxamer”, these copolymers are commonly named with the letter “P” (for poloxamer) followed by three digits, wherein the first two digits×100 represents the approximate molecular mass of the polyoxypropylene core, and the last digit×10 represents the percentage polyoxyethylene content (e.g., P407 is a poloxamer with a polyoxypropylene molecular mass of 4,000 g/mol and a 70% polyoxyethylene content). For the PLURONIC® and SYNPERONIC® trade names, coding of these copolymers starts with a letter to define its physical form at room temperature [L=liquid, P=paste, F=flake (solid)] followed by two or three digits. The first digit in a two-digit number, or the first two digits in a three-digit number, in the numerical designation, multiplied by 300, represents the approximate molecular weight of the hydrophobe; and the last digit×10 represents the percentage polyoxyethylene content (e.g., L61 indicates a polyoxypropylene molecular mass of 1,800 g/mol and a 10% polyoxyethylene content). In certain embodiments, poloxamer 181 (P181), PLURONIC® L61 and SYNPERONIC® PE/L 61 are interchangeable.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. Disease and disorder are used interchangeably herein.

As used herein, a “prophylactic” or “preventive” treatment is a treatment administered to a subject who does not exhibit signs of a disease or disorder or exhibits only early signs of the disease or disorder for the purpose of decreasing the risk of developing pathology associated with the disease or disorder.

As used herein, the term “RES” refers to reticulo-endothelial system.

By the term “specifically bind” or “specifically binds” as used herein is meant that a first molecule (e.g., an antibody) preferentially binds to a second molecule (e.g., a particular antigenic epitope), but does not necessarily bind only to that second molecule.

A “subject” or “individual” or “patient,” as used therein, can be a human or non-human mammal. Non-human mammals include, for example, livestock and pets, such as ovine, bovine, porcine, canine, feline and murine mammals. Preferably, the subject is human.

By the term “synthetic antibody” as used herein is meant an antibody generated using recombinant DNA technology, such as, for example, an antibody expressed by a bacteriophage as described herein. The term should also be construed to mean an antibody generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, a “therapeutic” treatment is a treatment administered to a subject who exhibits signs of pathology of a disease or disorder for the purpose of diminishing or eliminating those signs.

As used herein, the term “treating” means ameliorating the effects of, or delaying, halting or reversing the progress of a disease or disorder. The word encompasses reducing the severity of a symptom of a disease or disorder and/or the frequency of a symptom of a disease or disorder.

As used herein, the term “TWEEN®” is a trademark that refers to a polysorbate surfactant, which comprises a polyoxyethylene derivative of sorbitan fatty acid ester, wherein the length of the polyoxyethylene chain and the nature of the fatty acid may vary. Sorbitan is a mixture usually comprising 1,4-anhydrosorbitol, 1,5-anhydrosorbitol and 1,4,3,6-dianhydrosorbitol. Examples of this polysorbate surfactant include TWEEN® 20 and TWEEN® 21 (polyoxyethylene (20) sorbitan monolaurate), TWEEN® 40 (polyoxyethylene sorbitan monopalmitate), TWEEN® 60 and TWEEN® 61 (polyoxyethylene sorbitan monostearate), TWEEN® 65 (polyoxyethylene sorbitan tristearate), TWEEN® 80 (polyoxyethylene (20) sorbitan monooleate), and TWEEN® 85 (polyoxyethylene sorbitan trioleate).

Throughout this disclosure, various aspects of the present invention can be presented in a range format. It should be understood that the description in range format is merely for convenience and brevity and should not be construed as an inflexible limitation on the scope of the present invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

The invention includes a composition comprising at least one protein nanoparticle, wherein the protein nanoparticle is prepared by a method comprising the steps of: providing a solution comprising a protein and a non-ionic surfactant, wherein the concentration of the non-ionic surfactant in the solution ranges from about 5% to about 20,000% of the critical micelle concentration (CMC) of the non-ionic surfactant; and adjusting the pH of the solution to about the isoelectric point of the protein, thereby forming a precipitate comprising the protein nanoparticle, wherein the protein nanoparticle comprises the protein and the non-ionic surfactant.

In certain embodiments, the protein comprises a hormone, immunomodulator, cytokine, interferon, interleukin, or enzyme. In other embodiments, the protein comprises an antibody. In yet other embodiments, the protein comprises an immunoglobulin. In yet other embodiments, the immunoglobulin comprises IgA, IgD, IgE, IgG or IgM. In yet other embodiments, the immunoglobulin comprises IgG. In yet other embodiments, the antibody comprises a monoclonal antibody. In yet other embodiments, the monoclonal antibody comprises bevacizumab, anatumomab, benralizumab, enokizumab, mitumomab, oxelumab, palivizumab or any combinations thereof. In yet other embodiments, the non-ionic surfactant comprises an alkyl polyethylene oxide, an alkylphenol polyethylene oxide, a copolymer of polyethylene oxide and polypropylene oxide, an alkyl polyglucoside, a fatty alcohol, a cocamide MEA, a cocamide DEA, a polysorbate, or any combinations thereof. In yet other embodiments, the alkyl polyethylene oxide comprises diethylene glycol hexadecyl ether, polyethylene glycol oleyl ether, diethylene glycol octadecyl ether, polyoxyethylene stearyl ether, polyethylene glycol hexadecyl (cetyl) ether, polyethylene glycol dodecyl (lauryl) ether, decaethylene glycol oleyl ether, polyethylene glycol octadecyl ether, polyethylene glycol octadecyl ether, or any combinations thereof. In yet other embodiments, the average diameter of the at least one protein nanoparticle ranges from about 1 nm to about 1,000 nm. In yet other embodiments, the average diameter of the at least one protein nanoparticle ranges from about 10 nm to about 900 nm. In yet other embodiments, the average diameter of the at least one protein nanoparticle ranges from about 300 nm to about 600 nm. In yet other embodiments, the average diameter of the at least one protein nanoparticle ranges from about 250 nm to about 700 nm. In yet other embodiments, the concentration of the non-ionic surfactant in the solution ranges from about 5% to about 20,000% of the CMC of the surfactant. In yet other embodiments, the concentration of the non-ionic surfactant in the solution ranges from about 5% to about 100% of the CMC of the surfactant. In yet other embodiments, the concentration of the non-ionic surfactant in the solution ranges from about 10% to about 100% of the CMC of the surfactant. In yet other embodiments, the concentration of the non-ionic surfactant in the solution ranges from about 100% to about 20,000% of the CMC of the surfactant. In yet other embodiments, the concentration of the non-ionic surfactant in the solution ranges from about 300% to about 10,000% of the CMC of the surfactant. In yet other embodiments, the concentration of the non-ionic surfactant in the solution ranges from about 300% to about 5,000% of the CMC of the surfactant. In yet other embodiments, the composition further comprises a pharmaceutically acceptable carrier.

### Surfactants

Non-limiting examples of non-ionic surfactants useful within the compositions and methods of the present invention are alkyl polyethylene oxide (such as, but not limited to, diethylene glycol hexadecyl ether, polyethylene glycol oleyl ether, diethylene glycol octadecyl ether, polyoxyethylene stearyl ether, polyethylene glycol hexadecyl (cetyl) ether, polyethylene glycol dodecyl (lauryl) ether, decaethylene glycol oleyl ether, polyethylene glycol octadecyl ether, and polyethylene glycol octadecyl ether), alkylphenol polyethylene oxide, copolymers of polyethylene oxide and polypropylene oxide (known as poloxamers or poloxamines), alkyl polyglucosides (including octyl glucoside and decyl maltoside), fatty alcohols (including cetyl alcohol and oleyl alcohol), cocamide MEA, cocamide DEA, polysorbates (such as TWEEN® 20, TWEEN® 80 and dodecyl dimethylamine oxide).

### Ligands

In certain embodiments, the nanoparticles of the present invention further comprise at least one cell surface receptor ligand. In certain embodiments, the ligands allow for the nanoparticles of the present invention to recognize and bind to a cell that displays such cell surface receptor.

Non-limiting examples of ligands contemplated within the invention include ligands that bind to at least one of the following receptors: neurotensin receptor-1, human epidermal growth factor receptor-2 (HER-2), folate receptor, insulin-like growth (IGF) receptor, and/or epidermal growth factor receptor (EGFR).

Non-limiting examples of ligands contemplated within the invention include anti-NTSR1-mAb or SR-48692 (also known as 2-[[[1-(7-chloro-4-quinolinyl)-5-(2,6-dimethoxyphenyl)-1H-pyrazol-3-yl]carbonyl]amino]-tricyclo [3.3.1.13,7]decane-2-carboxylic acid), which bind to neurotensin receptor-1: trastuzumab, which binds to HER-2; folic acid (also known as (2S)-2-[[4-[(2-amino-4-oxo-1H-pteridin-6-yl)methylamino]benzoyl]amino]pentanedioic acid, N-(4-{[(2-amino-4-oxo-1,4-dihydropteridin-6-yl)methyl]amino}benzoyl)-L-glutamic acid; pteroyl-L-glutamic acid; Vitamin B9; or folacin), which binds to the folate receptor; anti-IGF-mAb (such as MK-0646, MA5-12247, AVE1642, figitumumab, or IMC-A12), which binds to the IGF receptor; and gefinitib, erlotinib, panitumumab, cetuximab, zalutumumab, nimotuzumab or matuzumab, which bind to the EGFR receptor.

### Antibodies

Antibodies are useful within the compositions and methods of the present invention. In certain embodiments, the antibody comprises IgG, bevacizumab, anatumomab, benralizumab, enokizumab, mitumomab, oxelumab, palivizumab and any combinations thereof within the methods of the present invention. In other embodiments, the antibody is human or humanized. In yet other embodiments, the antibody comprises an antibody selected from a polyclonal antibody, a monoclonal antibody, a humanized antibody, a synthetic antibody, a heavy chain antibody, a human antibody, and a biologically active fragment of an antibody.

Non-limiting examples of antibodies useful within the compositions and methods of the present invention include:
bevacizumab (AVASTIN®): humanized monoclonal antibody that inhibits vascular endothelial growth factor A (VEGF-A) and is used to treat cancers such as colon cancer, rectum cancer, lung cancer, glioblastoma, and renal cell cancer (Los et al., 2007, The Oncologist 12(4):443-50);
anatumomab mafenatox: a mouse monoclonal antibody for the treatment non-small cell lung cancer; a fusion protein of a Fab fragment with an enterotoxin (“mafenatox”) of S. aureus;
benralizumab: a monoclonal antibody for the treatment of asthma, and directed against the alpha-chain of the interleukin-5 receptor (CD125) (Catley, 2010, IDrugs: Invest. Drugs J. 13 (9):601-604);
enokizumab: a humanized monoclonal antibody designed for the treatment of asthma;
mitumomab (BEC-2): a mouse monoclonal antibody investigated for the treatment of small cell lung carcinoma;
oxelumab: human monoclonal antibody designed for the treatment of asthma; and,
palivizumab (SYNAGIS®): a monoclonal antibody used in the prevention of respiratory syncytial virus (RSV) infections; a humanized monoclonal antibody (IgG) directed against an epitope in the A antigenic site of the F protein of RSV.

It will be appreciated by one skilled in the art that an antibody comprises any immunoglobulin molecule, whether derived from natural sources or from recombinant sources, which is able to specifically bind to an epitope present on a target molecule.

In one aspect of the present invention, the target molecule is directly neutralized by an antibody that specifically binds to an epitope on the target molecule. In another aspect of the present invention, the effects of the target molecule are blocked by an antibody that specifically binds to an epitope on a downstream effector. In still another aspect of the present invention, the effects of the target molecule are blocked by an antibody that binds to an epitope of an upstream regulator of the target molecule.

When the antibody to the target molecule used in the compositions and methods of the present invention is a polyclonal antibody (IgG), the antibody is generated by inoculating a suitable animal with a peptide comprising full length target protein, or a fragment thereof, an upstream regulator, or fragments thereof. These polypeptides, or fragments thereof, may be obtained by any methods known in the art, including chemical synthesis and biological synthesis, as described elsewhere herein. Antibodies produced in the inoculated animal that specifically bind to the target molecule, or fragments thereof, are then isolated from fluid obtained from the animal.

Antibodies may be generated in this manner in several non-human mammals such as, but not limited to goat, sheep, horse, camel, rabbit, and donkey. Methods for generating polyclonal antibodies are well known in the art and are described, for example in Harlow et al., 1998, In: Antibodies, A Laboratory Manual, Cold Spring Harbor, N.Y.

Monoclonal antibodies directed against a full length target molecule, or fragments thereof, may be prepared using any well-known monoclonal antibody preparation procedures, such as those described, for example, in Harlow et al. (1998, In: Antibodies, A Laboratory Manual, Cold Spring Harbor, N.Y.) and in Tuszynski et al. (1988, Blood, 72:109-115). Human monoclonal antibodies may be prepared by the method described in U.S. Patent Publication No. 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al., 1992, Critical Rev. Immunol. 12(3,4):125-168, and the references cited therein.

When the antibody used in the methods of the present invention is a biologically active antibody fragment or a synthetic antibody corresponding to antibody to a full length target molecule, or fragments thereof, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology available in the art, and described, for example, in Wright et al., 1992, Critical Rev. in Immunol. 12(3,4):125-168 and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen et al. (U.S. Pat. No. 6,180,370), Wright et al., 1992, Critical Rev. Immunol. 12(3,4):125-168, and in the references cited therein, or in Gu et al., 1997, Thrombosis & Hematocyst 77(4):755-759, or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments typically include an expression control DNA sequence operably linked to humanized immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well-known procedures. Preferably, the human constant region DNA sequences are isolated from immortalized B-cells as described in International Patent Application Publication No. WO 198702671. CDRs useful in producing the antibodies of the present invention may be similarly derived from DNA encoding monoclonal antibodies capable of binding to the target molecule. Such humanized antibodies may be generated using well-known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., 1998, Antibodies: A Laboratory Manual, Cold Spring Harbor, N.Y.

V_{H} proteins isolated from other sources, such as animals with heavy chain disease (Seligmann et al., 1979, Immunological Rev. 48:145-167, incorporated herein by reference in its entirety), are also useful in the compositions and methods of the present invention. The present invention further comprises variable heavy chain immunoglobulins produced from mice and other mammals, as detailed in Ward et al., 1989, Nature 341:544-546 (incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as target molecule depletors in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA that specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., 2001, Molecular Cloning: A Laboratory Manual, Cold Spring Harbor Laboratory Press, Cold Spring Harbor, N.Y.

Bacteriophage that encode the desired antibody may be engineered such that the protein is displayed on the surface thereof in such a manner that it is available for binding to its corresponding binding protein, e.g., the antigen against which the antibody is directed. Thus, when bacteriophage that express a specific antibody are incubated in the presence of a cell which expresses the corresponding antigen, the bacteriophage will bind to the cell. Bacteriophage that do not express the antibody will not bind to the cell. Such panning techniques are well known in the art and are described for example, in Wright et al., 1992, Critical Rev. Immunol. 12(3,4):125-168.

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage that display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used to practice the method of the present invention, or to prepare a pharmaceutical composition useful in practicing the method of the present invention.

The antibodies of the present invention can be assayed for immunospecific binding by any method known in the art. The immunoassays that can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g, Current Protocols in Molecular Biology, (Ausubel et al., eds.), Greene Publishing Associates and Wiley-Interscience, New York (2002)). Exemplary immunoassays are described briefly below (but are not intended to be in any way limiting).

### Therapeutic Agents

In certain embodiments, the nanoparticles of the present invention further comprise at least one therapeutic agent. The at least one therapeutic agent may be a therapeutic, prophylactic, and/or diagnostic agent. Any suitable therapeutic agent may be used within the compositions and methods of the present invention. Non-limiting examples of therapeutic agent contemplated within the invention include organic compounds, inorganic compounds, hydrophobic or hydrophilic pharmacological drugs, radio pharmaceuticals, biologics, proteins, peptides, polysaccharides, nucleic acids, siRNA, RNAis, short hairpin RNAs (shRNAs), antisense nucleic acids), ribozymes, dominant negative mutants, or other materials that can be incorporated into the nanoparticles using standard techniques and/or the methods described herein.

In certain embodiments, the nanoparticles comprise an interfering RNA that reduces translation of at least one cell protein and/or polypeptide in a cell of a subject, wherein the cell protein is associated with a disease or disorder in the subject. An interfering RNA can include a siRNA, a shRNA, and a microRNA. An siRNA polynucleotide is an RNA nucleic acid molecule that interferes with RNA activity that is generally considered to occur via a post-transcriptional gene silencing mechanism. An siRNA polynucleotide preferably comprises a double-stranded RNA (dsRNA) but is not intended to be so limited and may comprise a single-stranded RNA (see, e.g., Martinez et al., 2002 Cell 110:563-74). The siRNA polynucleotide included in the invention may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′- to 3′-phosphodiester linkage). Accordingly, it will be appreciated that certain exemplary sequences disclosed herein as DNA sequences capable of directing the transcription of the siRNA polynucleotides are also intended to describe the corresponding RNA sequences and their complements, given the well-established principles of complementary nucleotide base-pairing.

Preferred siRNA polynucleotides comprise double-stranded polynucleotides of about 18-30 nucleotide base pairs, for example about 18, about 19, about 20, about 21, about 22, about 23, about 24, about 25, about 26, or about 27 base pairs, and in other embodiments about 19, about 20, about 21, about 22 or about 23 base pairs, or about 27 base pairs, whereby the use of “about” indicates that in certain embodiments and under certain conditions the processive cleavage steps that may give rise to functional siRNA polynucleotides that are capable of interfering with expression of a selected polypeptide may not be absolutely efficient. Hence, siRNA polynucleotides may include one or more siRNA polynucleotide molecules that may differ (e.g., by nucleotide insertion or deletion) in length by one, two, three, four or more base pairs as a consequence of the variability in processing, in biosynthesis, or in artificial synthesis of the siRNA. The siRNA polynucleotide of the present invention may also comprise a polynucleotide sequence that exhibits variability by differing (e.g., by nucleotide substitution, including transition or transversion) at one, two, three or four nucleotides from a particular sequence. These differences can occur at any of the nucleotide positions of a particular siRNA polynucleotide sequence, depending on the length of the molecule, whether situated in a sense or in an antisense strand of the double-stranded polynucleotide. The nucleotide difference may be found on one strand of a double-stranded polynucleotide, where the complementary nucleotide with which the substitute nucleotide would typically form hydrogen bond base pairing, may not necessarily be correspondingly substituted. In preferred embodiments, the siRNA polynucleotides are homogeneous with respect to a specific nucleotide sequence.

It should be appreciated that the siRNAs of the present invention may effect silencing of the target polypeptide expression to different degrees. Selection of siRNAs are made therefrom based on the ability of a given siRNA to interfere with or modulate the expression of the target polypeptide. The methods for testing each siRNA and selection of suitable siRNAs for use in the present invention are fully known to those skilled in the art. It is appreciated by one skilled in the art that siRNAs are easily designed and manufactured. Further, effects of siRNA are typically transient in nature, which make them optimal for certain therapies where sustained inhibition is undesired. Another form of an interfering RNA, shRNA polynucleotides utilize the endogenous processing machinery of the cell and are often designed for high potency, sustainable effects, and fewer off-target effects (Rao et al., 2009, Adv Drug Deliv Rev, 61: 746-759). As would be understood by those skilled in the art, the present invention encompasses both siRNA and shRNA polynucleotides, which can be designed and delivered to inhibit one or more cell proteins.

One skilled in the art will appreciate that one way to decrease the mRNA and/or protein levels of a cell protein is by reducing or inhibiting expression of the nucleic acid encoding the cell protein. Thus, the level of the cell protein in a cell can also be decreased using a molecule or compound that inhibits or reduces gene expression such as, for example, an antisense molecule or a ribozyme.

In a preferred embodiment, the modulating sequence is an antisense nucleic acid sequence expressed by a plasmid vector. The antisense expressing vector is used to transfect a mammalian cell or the mammal itself, thereby causing reduced endogenous expression of a desired protein in the cell. However, the invention should not be construed to be limited to inhibiting expression of a protein by transfection of cells with antisense molecules. Rather, the invention encompasses other methods known in the art for inhibiting expression or activity of a protein in the cell including, but not limited to, the use of a ribozyme, the expression of a non-functional protein (i.e. dominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

In another aspect of the present invention, the protein can be inhibited by way of inactivation and/or sequestration. As such, inhibiting the effects of a protein can be accomplished by using a dominant negative mutant. Alternatively an antibody specific for the desired protein, otherwise known as an antagonist to the protein, may be used. In certain embodiments, the antagonist is a protein and/or compound having the desirable property of interacting with a binding partner of the protein and thereby competing with the corresponding wild-type protein. In another embodiments, the antagonist is a protein and/or compound having the desirable property of interacting with the protein and thereby sequestering the protein.

Inhibition of one or more cell proteins can be accomplished using a modified nucleic acid molecule, such as a small interfering RNA (siRNA), short hairpin RNA (shRNA), a microRNA, an antisense nucleic acid, a ribozyme, an expression vector encoding a dominant negative mutant, and the likes. The methods of modifying nucleic acid molecules are known in the art. For example, a number of specific siRNA polynucleotide sequences useful for interfering with target polypeptide expression are known in the art. siRNA polynucleotides may generally be prepared by any method known in the art, including, for example, solid phase chemical synthesis. Modifications in a polynucleotide sequence may also be introduced using standard mutagenesis techniques, such as oligonucleotide-directed site-specific mutagenesis. Further, siRNAs may be chemically modified or conjugated with other molecules to improve their stability and/or delivery properties. Included as one aspect of the present invention are siRNAs as described herein, wherein one or more ribose sugars has been removed therefrom.

Alternatively, siRNA polynucleotide molecules may be generated by in vitro or in vivo transcription of suitable DNA sequences (e.g., polynucleotide sequences encoding a target polypeptide, or a desired portion thereof), provided that the DNA is incorporated into a vector with a suitable RNA polymerase promoter (such as for example, T7, U6, H1, or SP6 although other promoters may be equally useful). In addition, an siRNA polynucleotide may be administered to a mammal, as may be a DNA sequence (e.g., a recombinant nucleic acid construct as provided herein) that supports transcription (and optionally appropriate processing steps) such that a desired siRNA is generated in vivo.

In certain embodiments, an siRNA polynucleotide, wherein the siRNA polynucleotide is capable of interfering with expression of a target polypeptide can be used to generate a silenced cell. Any siRNA polynucleotide that, when contacted with a biological source for a period of time, results in a significant decrease in the expression of the target polypeptide is included in the invention. Preferably the decrease is greater than about 10%, more preferably greater than about 20%, more preferably greater than about 30%, more preferably greater than about 40%, about 50%, about 60%, about 70%, about 75%, about 80%, about 85%, about 90%, about 95% or about 98% relative to the expression level of the target polypeptide detected in the absence of the siRNA. Preferably, the presence of the siRNA polynucleotide in a cell does not result in or cause any undesired toxic effects, for example, apoptosis or death of a cell in which apoptosis is not a desired effect of RNA interference.

Any polynucleotide of the present invention may be further modified to increase its stability in vivo. Possible modifications include, but are not limited to, the addition of flanking sequences at the 5′ and/or 3′ ends; the use of phosphorothioate or 2′-O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

### Methods

The invention includes a method of preparing at least one protein nanoparticle. The method comprises providing a solution comprising a protein and a non-ionic surfactant, wherein the concentration of the non-ionic surfactant in the solution ranges from about 5% to about 20,000% of the critical micelle concentration (CMC) of the non-ionic surfactant. In certain embodiments, the solution further comprises at least one therapeutic agent. In other embodiments, the solution further comprises at least one cell surface receptor ligand. The method further comprises adjusting the pH of the solution to about the isoelectric point of the protein, thereby generating a precipitate comprising the at least one protein nanoparticle comprising the protein and the non-ionic surfactant. In certain embodiments, the precipitate further comprises the at least one therapeutic agent. In other embodiments, the precipitate further comprises the at least one cell surface receptor ligand. In yet other embodiments, the concentration of the non-ionic surfactant in the solution is greater than the critical micelle concentration (CMC) of the non-ionic surfactant.

In certain embodiments, the protein nanoparticle is further purified to remove protein or non-ionic surfactant that is not associated with the protein nanoparticle, to generate a composition comprising the at least one nanoparticle. In other embodiments, the composition comprising at least one nanoparticle is further lyophilized.

The invention further comprises a method of treating, ameliorating or preventing a disease or disorder in a subject in need thereof. The method comprises administering to the subject a pharmaceutically effective amount of a composition comprising at least one protein nanoparticle comprising a protein, a nonionic surfactant, optionally a therapeutic agent and optionally a cell surface receptor ligand, wherein the at least one protein nanoparticle is generated by precipitating the protein from a solution comprising the protein, the non-ionic surfactant, optionally the therapeutic agent and optionally the cell surface receptor ligand, wherein the concentration of the non-ionic surfactant in the solution ranges from about 5% to about 20,000% of the critical micelle concentration (CMC) of the non-ionic surfactant, further wherein the pH of the solution is adjusted to about the isoelectric point of the protein.

In certain embodiments, the composition is administered to the subject by an intrapulmonary, intrabronchial, inhalational, intranasal, intratracheal, intravenous, intramuscular, subcutaneous or topical route.

In certain embodiments, the composition further comprises a pharmaceutically acceptable carrier. In other embodiments, the subject is a mammal. In yet other embodiments, the mammal is human.

In certain embodiments, the protein comprises a hormone, immunomodulator, cytokine, interferon, interleukin, or enzyme. In other embodiments, the protein comprises an antibody. In yet other embodiments, the protein comprises an immunoglobulin. In yet other embodiments, the immunoglobulin comprises IgG. In yet other embodiments, the protein comprises an antibody. In yet other embodiments, the protein comprises an antibody. In yet other embodiments, the antibody comprises a monoclonal antibody. In yet other embodiments, the monoclonal antibody comprises bevacizumab, anatumomab, benralizumab, enokizumab, mitumomab, oxelumab, palivizumab or any combinations thereof. In yet other embodiments, the disease of disorder is selected from the group consisting of colon cancer, rectum cancer, lung cancer, glioblastoma, renal cell cancer, non-small cell lung cancer, small cell lung carcinoma, asthma, respiratory syncytial virus (RSV) infection, and any combinations thereof. In yet other embodiments, the non-ionic surfactant comprises an alkyl polyethylene oxide, an alkylphenol polyethylene oxide, a copolymer of polyethylene oxide and polypropylene oxide, an alkyl polyglucoside, a fatty alcohol, a cocamide MEA, a cocamide DEA, a polysorbate, or any combinations thereof. In yet other embodiments, the alkyl polyethylene oxide comprises diethylene glycol hexadecyl ether, polyethylene glycol oleyl ether, diethylene glycol octadecyl ether, polyoxyethylene stearyl ether, polyethylene glycol hexadecyl (cetyl) ether, polyethylene glycol dodecyl (lauryl) ether, decaethylene glycol oleyl ether, polyethylene glycol octadecyl ether, polyethylene glycol octadecyl ether, or any combinations thereof. In yet other embodiments, the average diameter of the at least one protein nanoparticle ranges from about 1 nm to about 1,000 nm. In yet other embodiments, the average diameter of the at least one protein nanoparticle ranges from about 10 nm to about 900 nm. In yet other embodiments, the average diameter of the at least one protein nanoparticle ranges from about 300 nm to about 600 nm. In yet other embodiments, the concentration of the non-ionic surfactant in the solution ranges from about 5% to about 20,000% of the CMC of the surfactant. In yet other embodiments, the concentration of the non-ionic surfactant in the solution ranges from about 5% to about 100% of the CMC of the surfactant. In yet other embodiments, the concentration of the non-ionic surfactant in the solution ranges from about 10% to about 100% of the CMC of the surfactant. In yet other embodiments, the concentration of the non-ionic surfactant in the solution ranges from about 100% to about 20,000% of the CMC of the surfactant. In yet other embodiments, the concentration of the non-ionic surfactant in the solution ranges from about 300% to about 10,000% of the CMC of the surfactant. In yet other embodiments, the concentration of the non-ionic surfactant in the solution ranges from about 300% to about 5,000% of the CMC of the surfactant.

### Pharmaceutical Compositions and Formulations

The invention also encompasses the use of pharmaceutical compositions of at least one composition of the present invention or a salt thereof to practice the methods of the present invention.

Such a pharmaceutical composition may consist of at least one composition of the present invention or a salt thereof, in a form suitable for administration to a subject, or the pharmaceutical composition may comprise at least one composition of the present invention or a salt thereof, and one or more pharmaceutically acceptable carriers, one or more additional ingredients, or some combination of these. The at least one composition of the present invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the present invention may be administered to deliver a API dose of between 1 ng/kg/day and 100 mg/kg/day, between 1 ng/kg/day and 500 mg/kg/day, or between 1 pg/kg/day and 10 ng/kg/day.

The relative amounts of the active ingredient, the pharmaceutically acceptable carrier, and any additional ingredients in a pharmaceutical composition of the present invention will vary, depending upon the identity, size, and condition of the subject treated and further depending upon the route by which the composition is to be administered.

Pharmaceutical compositions that are useful in the methods of the present invention may be suitably developed for inhalational, pulmonary, intranasal, intratracheal, intravenous, intramuscular, subcutaneous, topical, or another route of administration. Other contemplated formulations include projected nanoparticles, containing the active ingredient, and immunologically-based formulations. The route(s) of administration are readily apparent to the skilled artisan and depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose. The unit dosage form may also be for extended duration administration, such as once weekly or once monthly, depending on the efficacy of the protein formulation and the disease.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the present invention is contemplated include, but are not limited to, humans and other primates, mammals including commercially relevant mammals such as cattle, pigs, horses, sheep, cats, and dogs.

In certain embodiments, the compositions of the present invention are formulated using one or more pharmaceutically acceptable excipients or carriers. In certain embodiments, the pharmaceutical compositions of the present invention comprise a therapeutically effective amount of at least one composition of the present invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition.

Formulations may be employed in admixtures with conventional excipients, i.e., pharmaceutically acceptable organic or inorganic carrier substances suitable for nasal, inhalational, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents. As used herein, “additional ingredients” include, but are not limited to, one or more ingredients that may be used as a pharmaceutical carrier.

The composition of the present invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, and combinations thereof. A particularly preferred preservative is a combination of about 0.5% to 2.0% benzyl alcohol and 0.05% to 0.5% sorbic acid.

The composition preferably includes an antioxidant and a chelating agent that inhibit the degradation of the compound. Preferred antioxidants for some compounds are butylated hydroxytoluene (BHT), butylated hydroxyanisole (BHA), alpha-tocopherol (vitamin E) and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium ethylenediaminetetracetic acid (EDTA)) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

When used in vivo, the compositions of the present invention are preferably administered as a pharmaceutical composition, comprising a mixture, and a pharmaceutically acceptable carrier. The compositions of the present invention may be present in a pharmaceutical composition in an amount from 0.001 to 99.9 wt %, more preferably from about 0.01 to 99 wt %, and even more preferably from 0.1 to 95 wt %.

Administration of the compositions of the present invention to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the activity of the particular composition employed; the time of administration; the rate of excretion of the composition; the duration of the treatment; other drugs, compounds or materials used in combination with the composition; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the present invention is from about 0.01 μg/kg and 10 mg/kg of body weight/per day. One of ordinary skill in the art would be able to study the relevant factors and make the determination regarding the effective amount of the therapeutic composition without undue experimentation.

The composition can be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the present invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the present invention are dictated by and directly dependent on (a) the unique characteristics of the therapeutic composition and the particular therapeutic effect to be achieved, and (b) the limitations inherent in the art of compounding/formulating such a therapeutic composition for the treatment of diseases or disorders in a patient.

In certain embodiments, the compositions of the present invention are administered to the patient in dosages that range from one to five times per day or more. In other embodiments, the compositions of the present invention are administered to the patient in range of dosages that include, but are not limited to, once every day, every two, days, every three days to once a week, and once every two weeks. It will be readily apparent to one skilled in the art that the frequency of administration of the various combination compositions of the present invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compositions of the present invention for administration may be in the range of from about 1 μg to about 1,000 mg, about 2 μg to about 500 mg, about 4 μg to about 250 mg, about 6 μg to about 200 mg, about 8 μg to about 100 mg, about 10 μg to about 50 mg, about 20 μg to about 25 mg, about 40 μg to about 10 mg, about 50 μg to about 5 mg, about 100 μg to about 1 mg, and any and all whole or partial increments thereinbetween.

In some embodiments, the dose of a composition of the present invention is from about 0.5 μg and about 2,000 mg. In some embodiments, a dose of a composition described herein is less than about 2,000 mg, or less than about 1,000 mg, or less than about 500 mg, or less than about 250 mg, or less than about 100 mg, or less than about 50 mg, or less than about 25 mg, or less than about 10 mg, or less than about 5 mg, or less than about 1 mg, and any and all whole or partial increments thereof.

In certain embodiments, the present invention is directed to a packaged pharmaceutical composition comprising a container holding a therapeutically effective amount of a composition of the present invention, alone or in combination with a second pharmaceutical agent; and instructions for using the composition to treat, prevent, or reduce one or more symptoms of a disorder or disease in a patient.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a breathing disorder in a patient.

### Routes of Administration

Routes of administration of any of the compositions of the present invention include intrapulmonary, intrabronchial, inhalational, intranasal, intratracheal, intravenous, intramuscular, subcutaneous, topical, transdermal, oral, buccal, rectal, pleural, peritoneal, vaginal, epidural, otic, intraocular, or intrathecal administration.

Suitable compositions and dosage forms include, for example, suspensions, granules, beads, powders, pellets, and liquid sprays for nasal administration, dry powder or aerosolized formulations for inhalation, and the like. It should be understood that the formulations and compositions that would be useful in the present invention are not limited to the particular formulations and compositions that are described herein. For example, formulations may comprise a powder or an aerosolized or atomized solution or suspension comprising the active ingredient. Such powdered, aerosolized, or aerosolized formulations may further comprise one or more of the additional ingredients described herein. The examples of formulations described herein are not exhaustive and it is understood that the invention includes additional modifications of these and other formulations not described herein, but which are known to those of skill in the art.

### Controlled Release Formulations and Drug Delivery Systems

In certain embodiments, the formulations of the present invention can be, but are not limited to, short-term release or rapid-offset release, as well as controlled release, for example, sustained release, delayed release and pulsatile release formulations.

The term short-term or rapid-offset release is used in its conventional sense to refer to a drug formulation that provides for release of the drug immediately after drug administration.

As used herein, short-term or rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments there between after drug administration after drug administration.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be longer than the time required for the release of the same amount of agent administered in bolus form.

For sustained release, the compounds can be formulated with a suitable polymer or hydrophobic material that provides sustained release properties to the compounds. As such, the compounds of the present invention can be administered in the form of microparticles for example, by injection or in the form of wafers or discs by implantation.

In a preferred embodiment of the present invention, the compositions of the present invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

In a preferred embodiment of the present invention, the compositions of the present invention are administered to a subject, alone or in combination with another pharmaceutical agent, using a delayed release formulation.

The term pulsatile release is used herein in its conventional sense to refer to a drug formulation that provides release of the drug in such a way as to produce pulsed plasma profiles of the drug after drug administration.

In a preferred embodiment of the present invention, the compositions of the present invention are administered to a subject, alone or in combination with another pharmaceutical agent, using a pulsatile release formulation.

### Kits

The invention also includes a kit comprising a composition of the present invention and an instructional material that describes administering the composition to a mammal. As used herein, an “instructional material” includes a publication, a recording, a diagram, or any other medium of expression that can be used to communicate the usefulness of the composition of the present invention in the kit for effecting alleviation of the various diseases or disorders recited herein.

Optionally, or alternatively, the instructional material may describe one or more methods of alleviating the diseases or disorders in a cell or a tissue of a mammal. The instructional material of the kit may, for example, be affixed to a container that contains the invention or be shipped together with a container that contains the invention. Alternatively, the instructional material may be shipped separately from the container with the intention that the instructional material and the compound be used cooperatively by the recipient.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Experimental Details

### Materials & Cell Culture

Human polyclonal Immunoglobulin G1 (IgG1) was supplied in an excipient-free lyophilized form by Equitech-Bio, Texas, USA. Polysorbate 80 (Polyoxyethylene (80) sorbitan monooleate), polysorbate 20 (Polyoxyethylene (20) sorbitan monolaurate), and Brij 97 (polyethylene glycol monooleyl ether) were supplied by Sigma-Aldrich, Saint Louis, Mo. All other excipients and reagents were of reagent grades and were purchased from Fisher Scientific, Pittsburgh, Pa.

Poloxamer-188, 3-(4,5)-dimethylthiazol-3,5-di-phenytetrazoliumromide (MTT), RNase-free water, 4,6-diamidino-2-phenylindole (DAPI) and fetal bovine albumin (FBS) were obtained from Fisher Scientific. LysoTracker® Red, and Fluospheres® Red beads were purchased from Invitrogen.

Murine monoclonal anti-β-actin antibody was purchased from Sigma (MO, USA). siRNA against wild type KRAS was purchased from Santa Cruz Biotechnologies. siRNA against mutated KRAS G12S was designed and purchased from Thermo Scientific (formerly Dharmacon). siG12S sense and antisense sequences are GUUGGAGCUAGUGGCGUAGdTdT (SEQ ID NO:1) and CUACGCCACUAGCUCCAACdTdT (SEQ ID NO:2), respectively. Mouse TNFα ELISA kit was obtained from Thermo Scientific while Mouse IL-6 ELISA kit was obtained from BD Biosciences (USA). Lipopolysaccharide (LPS) from Escherichia coli was purchased from Sigma.

Adenocarcinoma cell line A549, expressing KRAS mutation at G12S and murine macrophage cell line (RAW 264.7) (ATCC TIB 71) were obtained from American Type Culture Collection (ATCC), Rockville, Md. A549 cells were maintained in F12K medium supplemented with 10% FBS and 1% antibiotics. RAW 264.7 cells were maintained in Dulbecco's Modified Eagle Medium (DMEM) supplemented with 10% FBS and 1% antibiotics. Both cells were kept in a humidified air atmosphere with 5% carbon dioxide.

### Methods

### Manufacture of Protein Nanoparticles

Nanoparticles were produced by dissolving different concentrations of the excipient-free human polyclonal IgG1 in 0.01N HCl containing different concentrations of polysorbate 80 (Tween 80), polysorbate 20 (Tween 20) or Brij 97. The concentrations of IgG used were 5, 7.5 and 10 mg/ml respectively. The mixture was then slowly titrated with 0.1N NaOH to bring the pH of the mixture to 7, which is the isoelectric point of human IgG (as provided by the suppliers) while continuously mixing on a magnetic stirrer. At the isoelectric point, the mixture became turbid, suggesting the precipitation of IgG nanoparticles. The colloidal suspension was then centrifuged using a microcentrifuge at 6500 rpm for 5 minutes. The supernatant was decanted and the pellet formed was rinsed with double distilled de-ionized water in order to remove any unprecipitated IgG and unattached surfactant micelles.

Lyophilized samples were prepared by resuspending the nanoparticles in water by vortexing. The suspended particles were then snap-frozen using liquid nitrogen before being loaded into freeze dryer (Labconco Freezone 4.6, Missouri). Lyophilization was performed for 24 hours. As control experiment, various concentrations of the surfactants used in the nanoprecipitation process were dissolved in 0.01N HCl and titrated to pH 7 using 0.1N NaOH. These were then used as controls in all the analytical procedures.

### Percentage Yield of Nanoparticles

The percentage yield of nanoparticles produced from the nanoprecipitation process was determined by taking samples from the supernatant following centrifugation and analyzing for protein content using UV absorption at 280 nm. % yield was calculated as thus:

100×[Total amount of IgG−unprecipitated IgG]/[Total amount of IgG]

### Photon Correlation Spectroscopy (PCS)

Particle size (by intensity) and zeta potential measurements were performed by PCS using Zetasizer Nano ZS (Malvern Instruments, U.K.). The pellets formed after centrifugation at 6,500 rpm for 5 min were thoroughly rinsed and resuspended in deionized water by vortexing. The samples were then sonicated for approximately 5 min. Intensity autocorrelation was measured at a scattering angle (θ) of 173° at 25° C. The Z-average and polydispersity index (PDI) were recorded in triplicate. For zeta potential measurement, the samples were loaded into a universal dip cell (Malvern Instruments, U.K.) before recording the zeta potential in triplicate.

### Particle Size Analysis by Dynamic Light Scattering (DLS)

The particle size analysis (by intensity) of the nanoparticles was performed by dynamic light scattering (DLS) using Zetasizer Nano ZS (Malvern, UK). The pellets formed after centrifugation at 6500 rpm for 5 minutes were thoroughly rinsed and resuspended in deionized water by vortexing. The samples were then sonicated for approximately 5 minutes. Intensity autocorrelation was measured at a scattering angle (θ) of 173 degrees at 25° C. The control samples (surfactants alone) described in the methods section were also analyzed for particle size to determine whether the presence of micelles were interfering with the measurement. The Z-average and polydispersity index (PDI) were recorded in triplicate.

### Scanning Electron Microscopy (SEM)

The morphology of the manufactured nanoparticles and the unprocessed IgG was observed by scanning electron microscopy using the Zeiss Supra 50VP system (Zeiss, Germany). Powders were mounted onto aluminum stubs using double sided adhesive tape and were made electrically conductive by coating in a thin layer of gold. The coated samples were then examined under microscope operated at an acceleration voltage of 5 kV.

### Protein Content by Absorbance at 280 nm

The IgG content in the particles formed was determined by constructing a standard calibration curve with unprocessed IgG with concentrations ranging from 0-3 mg/ml in 0.1M acetate buffer (pH 5). The IgG was confirmed to totally dissolve in the buffer by monitoring for absence of any particles under light microscopy. The UV absorbance of these solutions was measured at 280 nm. 1 mg/ml solutions of the dissolved nanoparticles were prepared and their “actual” concentrations determined by UV spectroscopy using the constructed calibration curve. IgG was determined as a percentage ratio of the “actual” concentrations to the theoretical concentrations (1 mg/ml). The experiment was repeated in triplicate for each sample.

### IgG Specific Binding Activity

ELISA was performed using a human IgG quantitation kit from Bethyl Laboratories Inc (Texas). Briefly, human IgG present in the freeze dried samples (250 ng/mL) was captured by the anti-human IgG antibody which was pre-coated on the surface of the microtiter wells. The system was then incubated for 1 hour. After sample binding, any unbound protein in the plate was washed off four times with the wash buffer. Biotinylated detection antibody was then added to the wells to bind to the captured human IgG and incubated at room temperature. After washing four times, 100 μL of horseradish peroxidase (HRP) is then added to catalyze the colorimetric reaction with the chromogenic substrate 3,3′,5,5′-tetramethylbenzidine (TMB). The reaction produced a blue colored product in dark, which turned yellow when the reaction was terminated by the addition of dilute sulfuric acid. The absorbance of the yellow product was measured at 450 nm using a SpectraMax 340 (Molecular Devices, Sunnyvale, Calif.). IgG standard curve were used in the 500-0.69 ng/mL range and were prepared by diluting the stock solution (500 ng/mL) with the dilution buffer provided by the supplier. Each sample was assayed three times (n=3) and all reactions were carried out at room temperature. The control samples (surfactants alone) described in the methods section were also assayed to determine whether they interfered with the binding of IgG.

### SE-HPLC

SE-HPLC was performed using an Alliance HPLC System, Waters 2695 separation module (Waters, Mass., USA) combined with a Waters 2998 Photo-diode Array Detector. A TSK Gel 3000 SWXL column (300 mm×7.8 mm) (Tosoh Coperation, OH, USA) was used. 20 μl of the nanoparticles, dissolved in 0.1M acetate buffer (pH 5) at 1 mg/ml was injected and separation was performed at a flow rate of 0.5 ml/min using 0.2 M sodium phosphate (pH 6.0) as the mobile phase. UV detection was performed at 214 nm. Chromatograms were recorded using the Empower Pro® software. The control samples (surfactants alone) described in the methods section were also analyzed to see whether they interfere with the UV detection at 214 nm.

### Far-UV Circular Dichroism (CD) Spectroscopy

CD measurements were performed with Jasco J-810 Spectropolarimeter (Jasco, Md., USA) operating at 20° C. using 0.5 mg/ml of reconstituted solutions of IgG nanoparticles in acetate buffer. CD spectra were obtained in the far UV region (260-190 nm) using a quartz cell of 0.1 path length in order to probe the stability of the secondary structure of the manufactured nanoparticles. A scanning speed of 50 nm/min with a 0.5-second response time was applied followed by five accumulations for each sample. The experiment was repeated in triplicate for each sample. Surfactants dissolved in the acetate buffer at corresponding concentrations were used as blanks. Signals from blanks were subtracted from sample signals. The CD signals were converted to mean residue weight ellipticity and the percentage of the secondary structure retained was estimated using the K2D2 software.

### In Vitro Deposition Studies Using the Andersen Cascade Impactor (ACI)

The in vitro deposition patterns of the IgG nanoparticles were assessed using the eightstage ACI (Copley Scientific, Nottingham, UK) as previously described (22). ACI is an eight-stage device wildly used for assessing the lung deposition of aerosols. As illustrated in FIG. 20, it mimics the respiratory tract and gives an indication of how and where the aerosol particles/droplets will be deposited in the respiratory airways. Aerosols with mass median aerodynamic diameter of 0.5-3 μm have been widely reported as optimum for deep lung delivery. The fine particle fraction (FPF) of an aerosol gives an indication of the fraction of the dose of a drug that gets to the peripheral lung (terminal bronchi—alveoli). Most aerosols on the market usually have FPF less than 50%. In this study, two dry powder devices: Handihaler® (used in Spiriva®) and Spinhaler®. These two devices were used because of the differences in their resistance.

The flow rate (Q) through the ACI was set to 30 l/min using a flow meter model DFM2000 (Copley Scientific, Nottingham. UK). The pressure drop was set at 4 kPa. A 41 inspiration volume was achieved by setting the timer so that t=[4*60/Q] s.

Using both Handihaler® (Boehringer Ingelheim, Germany) and Spinhaler® (Fison Pharmaceuticals, UK) as model devices, IgG powders containing an equivalent of 1 mg IgG nanoparticles were filled into the respective capsules before being loaded into the devices. The content of the capsule was then sampled by the ACI before collection of the plates on each stage and washing with 0.1M acetate buffer (pH 5). For each formulation, a total of 6 capsules were sampled. The content of IgG on each plate was quantified by UV absorption at 280 nm.

The recovered dose was calculated as the total amount of drug recovered from the device, capsule and the 8-stage impactor. The analysis was accepted if this fell within 75%-125% of the nominal loaded dose.

A table of cumulative mass % (as % of the total recovered emitted dose) versus effective cut-off diameter for each stage of the impactor was constructed. Fine particle fraction (FPF) was defined as the amount of IgG particles with aerodynamic diameter<5 μm. Analyses were run in triplicate and the data expressed as mean±standard deviation.

### Retained In Vitro Anti-VEGF Activity

The in vitro anti-VEGF activity assay was adapted from Wang et al., 2004, Angiogenesis 7:335-345. Briefly, HUVECs grown at 80% confluence were harvested and seeded in 2×96 well plates at 2×10⁵ cells/well in ice-cold endothelial basal growth medium (serum free) with no growth factors and FBS supplementation. 50 μL of a wide concentration range of rhVEGF (0-1000 ng/mL) was added into designated wells in four replicates. Cells in complete growth medium were used as positive control to assess HUVEC proliferation. For bevacizumab inhibition, distinct concentrations of bevacizumab (0-500 ng/mL) were mixed with 50 ng/mL of rhVEGF and incubated at 37° C. in a humidified air atmosphere with 5% carbon dioxide for 2 h prior to adding the cell suspension. The plate was continuously incubated for 4 days. At the end of incubation, 25 μL of alamarBlue (Sigma Aldrich, Mo.) was added to each well and incubated for an additional 6 h under same conditions. The plate was then read at 530/590 nm excitation/emission on a fluorescence plate reader. The alamarBlue dye is a fluorometric growth indicator based on metabolic activity, which is reflective of extent of cellular proliferation. The control sample contained cells deprived of FBS and growth factors for 3 days. A non-specific human IgG was also tested to exclude any possibility of a non-specific effect mediated by IgG. A control sample consisting of surfactants dissolved in the growth medium was also tested to exclude any anti-VEGF effect from the surfactants.

### Cytotoxicity Assay

Cytotoxic effect of bevacizumab was assessed in adenocarcinoma cells (A549) using MTT assay. Cells (1×10⁴ cells/well) were seeded in 96 well plates and incubated at 37° C. in a humidified air atmosphere with 5% carbon dioxide for 48 h. The cells were treated with varying concentrations of unprocessed bevacizumab particles and reconstituted bevacizumab nanoparticles (0-1000 μg/mL) and incubated for 72 h. At the end of incubation (after 72 h), MTT reagent was added to each well and incubated for 3 h.

The medium was aspirated, and 100 μL of DMSO was added. The plate was read at 560 nm. Cells treated with DMSO and relevant surfactants were used as a control.

### Fluorescence Microscopy

A549 and MRC-5 cells (2×10⁴ cells/well) were seeded in 8 well coated glass slides and incubated for 48 h. PBS washed cells were incubated with FITC-labeled bevacizumab particles suspended in the serum-free medium (100 μg/mL) for 60 min. Cells were washed with PBS, fixed with 2% paraformaldehyde, and incubated at room temperature for 20 min. PBS washed cells were then blocked with 5% BSA for 30 min at room temperature. Cells were stained with AlexaFluoro-555 labeled wheat germ agglutinin (WGA) and 4′,6-diamidino-2-phenylindole (DAPI) to visualize plasma membrane and nucleus respectively. Cells were mounted and observed under a Leica DMI 6000B fluorescence microscope (Leica Microsystems, Exton, Pa.).

### Uptake Experiments

A549 and MRC-5 cells were used to investigate the uptake of self-associated bevacizumab nano-particles. About 1 million cells/well were seeded in a 12 well plate and incubated for 48 h. Cells were then treated with 100 μg/mL the FITC conjugated nanoparticles resuspended in serum-free medium and incubated for 60 min. The cells were trypsinized and centrifuged at 300 g for 5 min, and the pellet was washed and resuspended in 0.4% trypan blue (TB) solution in PBS to quench the extracellular FITC fluorescence. TB, while quenching the FITC fluorescence of non-internalized particles, causes them to fluoresce red whereas an internalized particle will fluoresce green. Cells were then centrifuged; the TB solution was removed before the cell pellets were resuspended in PBS. The samples were then analyzed by flow cytometry (BDFACS caliber). 10,000 cells were measured in each sample.

To elucidate whether the uptake of bevacizumab nanoparticles was due to specific interaction with the cell membrane or not, A549 cells incubated in medium containing 10% FBS were treated with 100,000 μg/mL unlabeled bevacizumab 60 min prior to the treatment with 100 μg/mL FITC-labeled bavacizumab nanoparticles. Mean fluorescence intensity obtained from this experiment was compared to that obtained when the A549 cells were treated with 100 μg/mL FITC-labeled bavacizumab nanoparticles alone.

For the elucidation of the mechanisms of internalization, cells were preincubated for 60 min at 37° C./5% carbon dioxide with 2 μg/mL nocodazole, 0.1% sodium azide/50 mM deoxy glucose, 75 μM dynasore, and 2 μg/mL filipin before being treated with the nanoparticles.

### Transmission Electron Microscopy (TEM)

Visual observation of particle internalization and translocation was gained using TEM. Approximately 5×10⁵ cells (A549) were seeded in 60 mm² polystyrene dishes for 48 h. PBS washed cells were treated with 100 μg/mL bevacizumab nanoparticles and incubated at the same time. However, they were fixed at distinct time points: 15 min, 60 min, and 4 h. At the end of each time point, the cells were fixed in 2% gluteraldehyde with 1% tannic acid in 0.05 M phosphate buffer. The cells were rinsed three times in 0.1 M phosphate buffer. The cells were postfixed in 2% osmium tetroxide in 0.1 M phosphate buffer. After being rinsed in deionized water, the cells were stained with 1% uranyl acetate in deionized water. Cells were spun down in warm agarose, and the cell pellet was dehydrated in graded steps of acetone and infiltrated with Spurr's embedding medium. The blocks were polymerized at 65° C. in a convection oven. The resulting blocks were cut with a Diatome diamond knife on a Leica Ultracut UCT microtome. The thin sections were picked up with copper grids and observed in a FEI Tecnai 12 TEM. Electron micrographs were captured with an AMT XR111 11 megapixel CCD camera.

### Statistical Analysis

Results are expressed as mean±standard deviation, unless otherwise indicated. Statistically significant difference between two groups was determined by two-tailed Student's t test. A p-value of 0.05 was taken as statistically significant.

### Example 1

### Nanoparticles

### Percentage Yield of Nanoparticles

The percentage nanoparticle yield was neither affected by the type nor concentration of the surfactant. The yields from all the preparations were approximately 85% irrespective of the type of the surfactant and the concentration of IgG in the precipitating medium.

### Particle Size Analysis

DLS data in Table 1 revealed that the particles that were produced using this technology were in a size ranging from approximately 90-800 nm. Changes in the sizes of the nanoparticles were observed based on the type and concentration of the surfactant used. Table 1 indicates that the highest diameter (795.7 nm) was produced by nanoparticles precipitated from the surfactant-free 5 mg/ml IgG solution (negative control). Upon the addition of Tween 80 to the precipitating medium, a concentration dependent variation in size was observed. The smallest particles precipitated in the presence of Tween 80 from 5 mg/ml solution was when 0.1% w/v Tween 80 was added to the precipitating solution (181.9 nm) while the biggest particles were produced in the presence of 0.3% w/v Tween 80 (265.3 nm). A more or less concentration-dependent variation in particle size was also observed when Tween 80 was replaced with Tween 20 in the precipitating solution. When 0.1% w/v Tween 20 was added to the precipitating medium, particles 403.5 nm in diameter were produced. In the presence of 0.2% w/v Tween 20 the particle size increased to 493.7 nm. The particle size stayed at 493 nm when the concentration of the Tween 20 added was increased to 0.3% w/v.

When the concentration of the IgG in the precipitating solution was increased to 7.5 mg/ml, a concentration-dependent variation in particle size was also observed when the nanoprecipitation was performed in the presence of Tween 80 (Table 1). Nanoprecipitation from 7.5 mg/ml IgG solution in the presence of 0.1, 0.2 and 0.3% w/v Tween 20 produced nanoparticles 132.5, 151.8 and 187.2 nm in diameter respectively, suggesting an increase in particle size as the concentration of the surfactant in the precipitating medium increased. Further, particles precipitated in the presence of Tween 20 from 7.5 mg/ml solutions had particles size lower than what was observed when the particles were produced from 5 mg/ml IgG solutions.

Table 1 indicated that as the concentration of Tween 20 in the 7.5 mg/ml IgG solution increased, the size of the particles produced increased from 171.6 to 197.5 nm. The size of nanoparticles precipitated from the 10 mg/ml IgG solution is illustrated in Table 1. 1. While the particles precipitated in the presence of Tween 20 showed a similar trend to that of the corresponding particles precipitated from 5 mg/ml and 7.5 mg/ml, i.e., an increasing particles size with an increase in Tween 20 concentration, the particles produced using this concentration were generally smaller than the particles produced from corresponding 5 and 7.5 mg/ml IgG solutions.

TABLE 1
Particle size analysis by DLS for particles precipitated from IgG solutions.

| | 5 mg/ml IgG solution | | 7.5 mg/ml IgG solution | | 10 mg/ml IgG solution | |
|---|---|---|---|---|---|---|
| | Particle | | Particle | | Particle | |
| | Diameter | | Diameter | | Diameter | |
| Sample | (nm)* | PDI* | (nm)* | PDI* | (nm)* | PDI* |
| Particles precipitated with | 181.9 ± 4.9 | 0.1 ± 0.0 | 132.5 ± 4.6 | 0.1 ± 0.1 | 110.8 ± 5.6 | 0.6 ± 0.1 |
| 0.1% Tween 80 | | | | | | |
| Particles precipitated with | 220.7 ± 3.7 | 0.1 ± 0.2 | 151.8 ± 34.2 | 0.1 ± 0.1 | 131.9 ± 4.1 | 0.6 ± 0.0 |
| 0.2% Tween 80 | | | | | | |
| Particles precipitated with | 265.3 ± 2.3 | 0.1 ± 0.3 | 187.2 ± 1.7 | 0.1 ± 0.1 | 188.0 ± 6.9 | 0.5 ± 0.0 |
| 0.3% Tween 80 | | | | | | |
| Particles precipitated with | 403.5 ± 1.4 | 0.2 ± 0.0 | 171.6 ± 6.0 | 0.2 ± 0.1 | 106.5 ± 1.4 | 0.5 ± 0.0 |
| 0.1% Tween 20 | | | | | | |
| Particles precipitated with | 493.7 ± 0.8 | 0.1 ± 0.2 | 183.7 ± 9.0 | 0.1 ± 0.0 | 160.1 ± 8.3 | 0.6 ± 0 1 |
| 0.2%Tween 20 | | | | | | |
| Particles precipitated with | 493.1 ± 4.5 | 0.1 ± 0.0 | 197.5 ± 11.2 | 0.1 ± 0.0 | 185.4 ± 2.4 | 0.5 ± 0.1 |
| 0.3%Tween 20 | | | | | | |
| Particles precipitated with | 492.6 ± 7.9 | 0.2 ± 0.1 | 489.7 ± 1.4 | 0.2 ± 0.0 | 94.2 ± 5.7 | 0.3 ± 0.1 |
| 0.1% Brij 97 | | | | | | |
| Particles precipitated with | 522.1 ± 2.0 | 0.1 ± 0.0 | 558.5 ± 6.5 | 0.1 ± 0.0 | 187.6 ± 0.7 | 0.4 ± 0.1 |
| 0.2% Brij 97 | | | | | | |
| Particles precipitated with | 535.7 ± 3.6 | 0.1 ± 0.1 | 563.9 ± 3.4 | 0.1 ± 0.1 | 243.3 ± 4.0 | 0.7 ± 0.1 |
| 0.3% Brij 97 | | | | | | |
| Particles precipitated | 795.7 ± 8.8 | 0.1 ± 0.1 | 774.9 ± 8.2 | 0.1 ± 0.2 | 741.3 ± 7.0 | 0.4 ± 0.0 |
| without surfactant | | | | | | |
| (negative control) | | | | | | |

*Data presented as Mean ± SD, n = 3

Generally, particles produced in the presence of Brij 97 were bigger than particles produced in the presence of the Tweens from all IgG solutions. The control samples showed no peaks nor reported any particle size after measurement.

### Particle Morphology

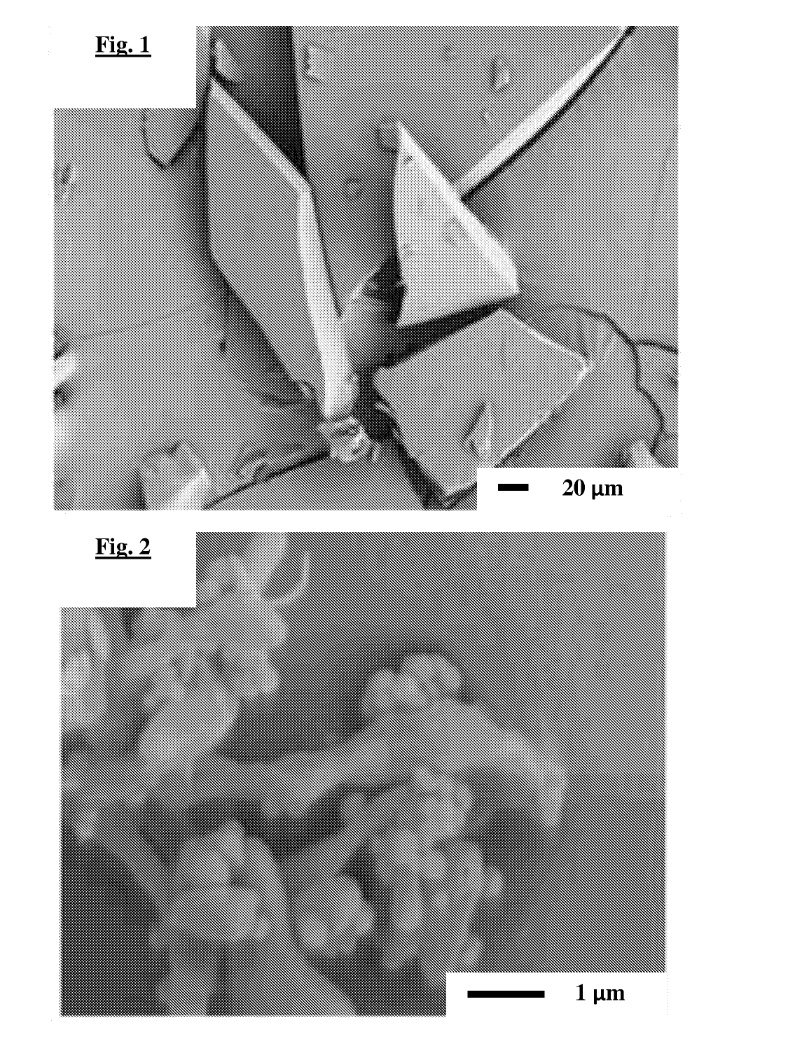

As illustrated in FIG. 1, the unprocessed IgG particles as received from the supplier were irregularly shaped. The scale bar in the micrograph suggests that the particles are many folds bigger than 20 μm.

FIG. 2 illustrates the SEM micrograph of the particles precipitated from the surfactant-free 5 mg/ml IgG solution. The particles were generally spherical in shape. Variations in the morphology of the particles were observed when the precipitation was performed in the presence of different surfactants as illustrated in FIGS. 3-5. By keeping the concentrations of the IgG and surfactants in the solutions at 5 mg/ml and 0.1% w/v respectively, the particles formed different shapes depending on the type of surfactant used.

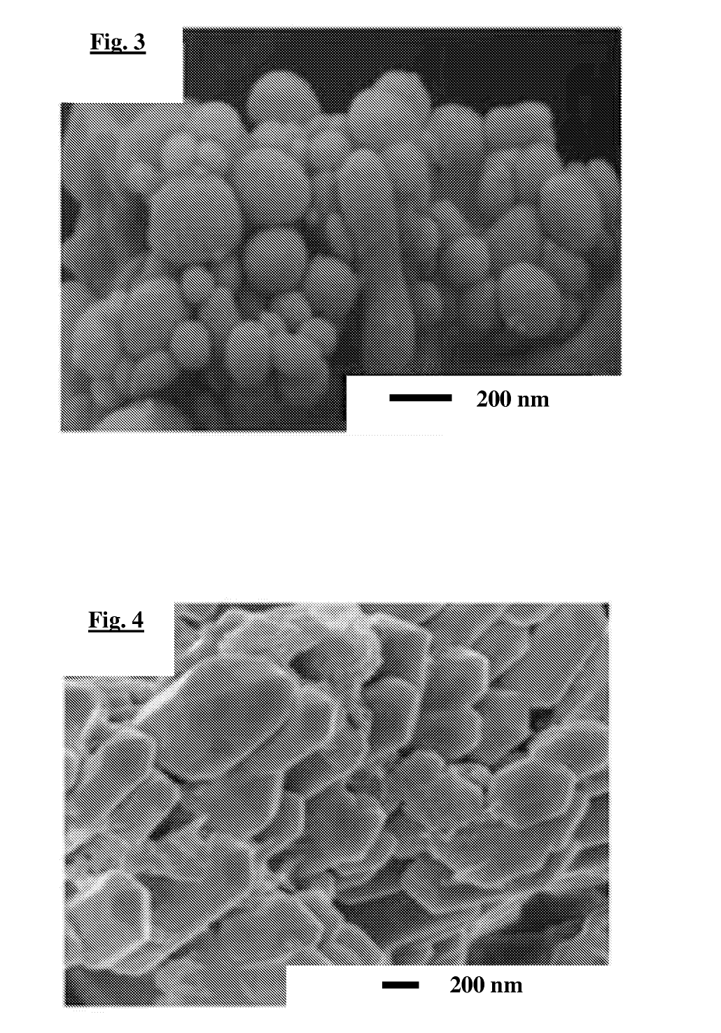

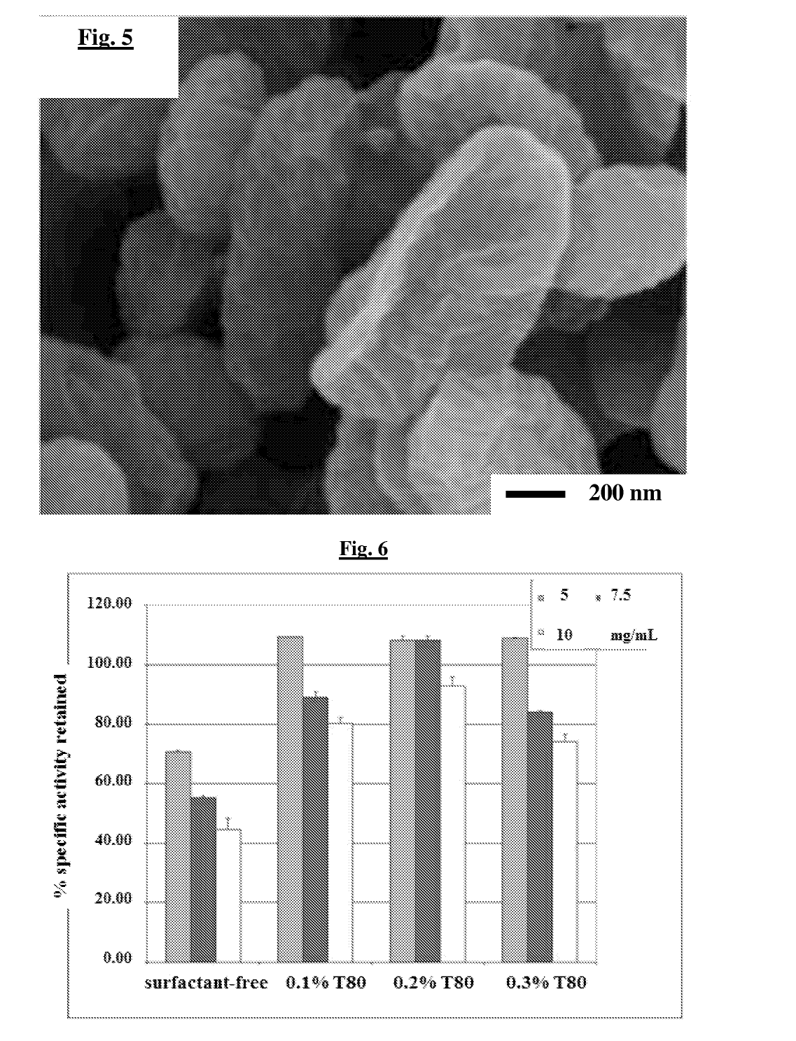

As illustrated in FIG. 3, spherical particles were formed when Tween 80 was added to the precipitating solution. FIG. 4 reveals that particles precipitated from the 5 mg/ml IgG solution in the presence of 0.1% w/v Tween 20 were polyhedral in shape. They also appear agglomerated although the SEM image suggests that they can be easily de-agglomerated with little agitation. Particles precipitated from the 5 mg/ml IgG solution in the presence of 0.1% w/v Brij 97 appeared sponge-like and were de-agglomerated (FIG. 5). A conspicuous effect of the type of surfactant present in the precipitating medium on the shape of the particles produced can be clearly seen in the SEM images in FIGS. 3-5.

### IgG Content in Nanoparticles

The amount of IgG in the nanoparticles as determined by UV absorption at 280 nm was found to vary between 92-101%.

### IgG Specific Binding Activity

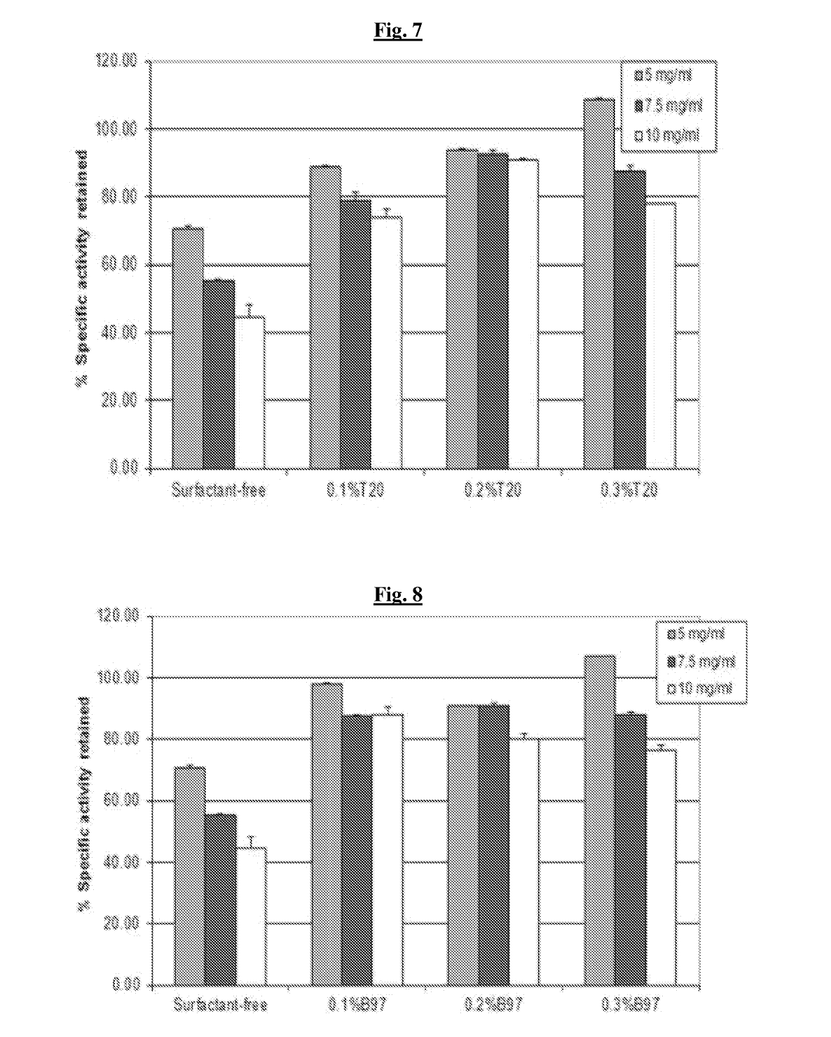

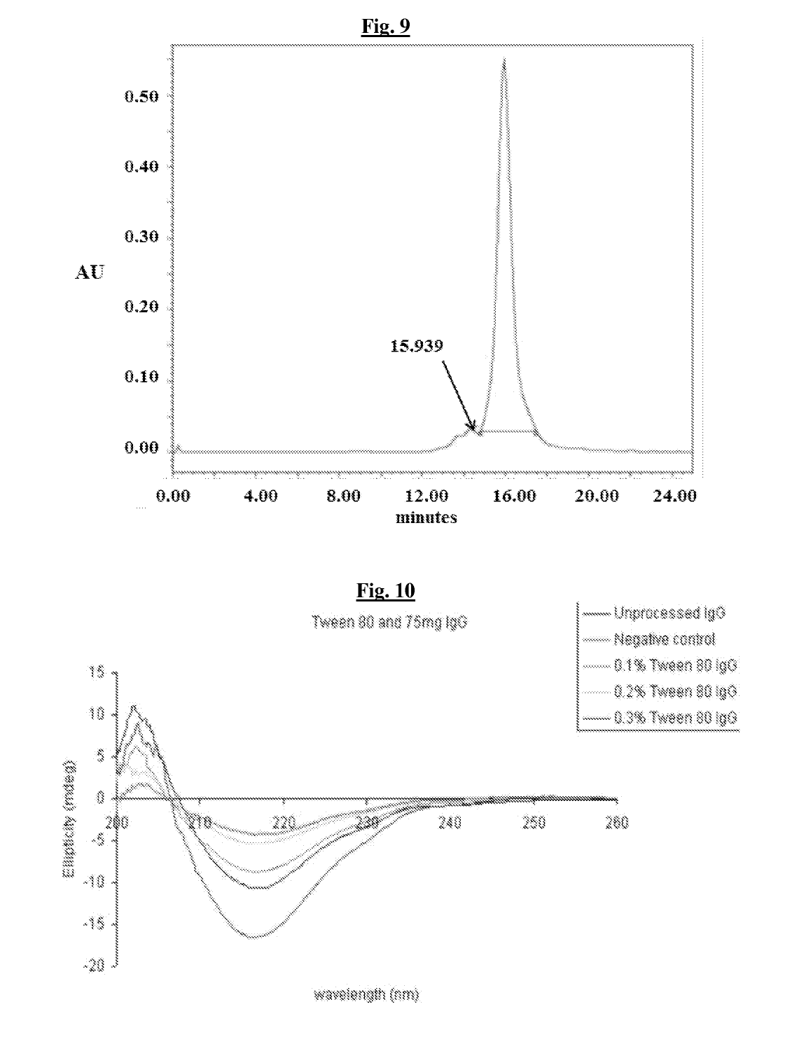

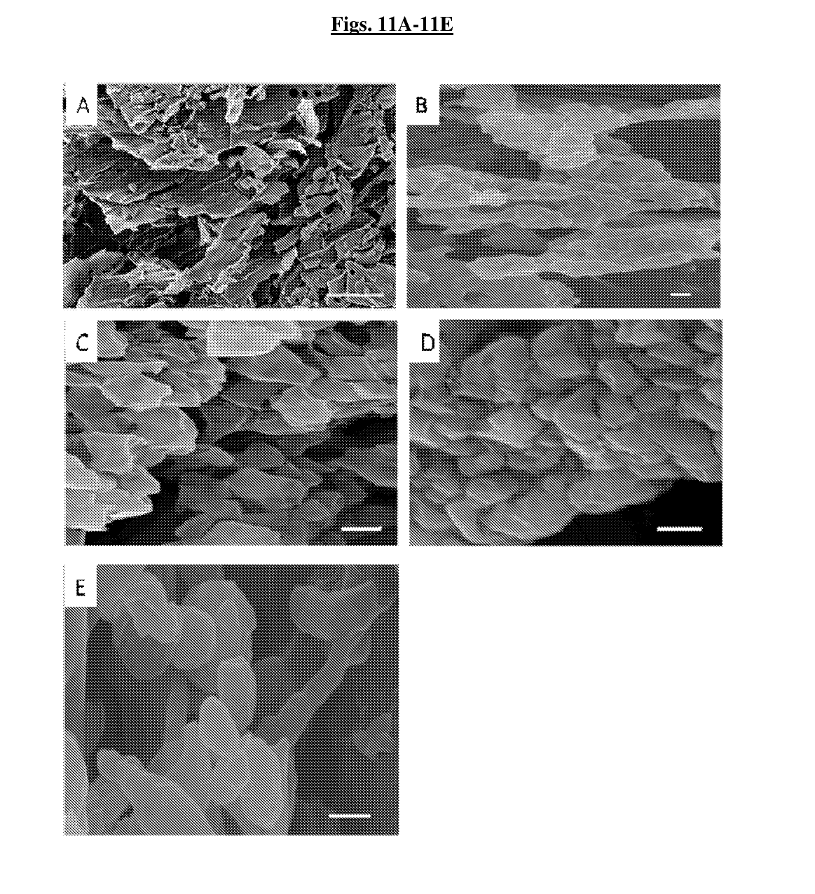

The specific binding affinity of the IgG nanoparticles was determined by ELISA as described elsewhere herein. This procedure was adapted from a previously described method by Dani et al., 2007, J. Pharm. Sci. 96:1504-1517. FIGS. 6-8 illustrate how the percentage specific binding activity retained by the IgG nanoparticles as fraction of the unprocessed IgG. Nanoparticles precipitated from surfactant free IgG solutions retained the lowest binding activity in comparison to nanoparticles precipitated from the corresponding surfactant containing solutions. The binding affinity retained by nanoparticles precipitated from surfactant-free (negative control) 5 mg/ml IgG solution was approximately 70% (FIG. 6). However, nanoparticles precipitated from corresponding IgG solutions containing 0.1, 0.2 and 0.3% Tween 80 retained approximately 109, 108 and 106% binding activity, suggesting that the presence of the Tween 80 in the precipitating solutions contributed to the retention of binding activity in the IgG nanoparticles. Similar trends were observed in the nanoparticles precipitated from solutions containing Tween 20 and Brij 97 (FIGS. 7-8). For all the nanoparticles, irrespective of the concentrations and the type of surfactant used, nanoparticles precipitated from 5 mg/ml generally had superior binding activity in comparison to the corresponding nanoparticles precipitated from 7.5 and 10 mg/ml. In FIG. 6, nanoparticles precipitated from the 5 mg/ml IgG solution containing 0.1% Tween 80 retained bioactivity of approximately 109% while nanoparticles precipitated from corresponding solutions containing 7.5 mg/ml and 10 mg/ml showed a retained binding activity of 90 and 80% respectively. No binding was observed in the control samples.

### SE-HPLC

The presence of monomers, soluble aggregates and fragments in the reconstituted IgG nanoparticles was investigated using SE-HPLC. FIG. 9 shows the SE-HPLC chromatogram of the unprocessed IgG. The peak at retention time of approximately 16 minutes represents the intact IgG monomer. The minor peaks at approximately 13 and 14 minutes suggest the presence of low levels of aggregates in the reconstituted unprocessed IgG. Peaks from all the reconstituted nanoparticles were similar to those seen in the unprocessed IgG. The control samples showed no discernable peaks. The percentage monomer recovered from each nanoparticle formulation following reconstitution was determined and compared to that of the unprocessed IgG. Tables 2-4 illustrate the percentages of the monomer recovered by SE-HPLC analysis. The percent monomer recovered from nanoparticles precipitated from 5 mg/ml IgG solutions ranged from 73 to 98% and 63 to 93% from 7.5 mg/ml solutions, while particles from 10 mg/ml IgG solutions had between 61 and 90% monomer recovery. SE-HPLC data suggest that nanoparticles precipitated from 5 mg/ml IgG solutions had superior monomer recovery in comparison to the corresponding nanoparticles precipitated from 7.5 and 10 mg/ml solutions. Differences in concentrations of the surfactants did not seem to have an obvious effect on the percent monomer retained. However, the total relative recovery generally decreased as the concentration of the IgG solutions from which the nanoparticles were precipitated from increased.

TABLE 2
SE-HPLC analysis of reconstituted IgG nanoparticles produced from 5 mg/ml
solutions.

| | Handihaler | | | | Spinhaler | | | |
|---|---|---|---|---|---|---|---|---|
| | FPF^{a} | MMAD^{b} | | ED^{d} | FPF | MMAD | | |
| Particles | (%) | (μm) | GSD^{c} | (%) | (%) | (μm) | GSD | ED |
| Unprocessed | 20 ± 4 | 8.1 ± 0.4 | 3.2 ± 0.2 | 97 ± 2 | 22 ± 2 | 8.5 ± 0.3 | 2.2 ± 0.3 | 95 ± 3 |
| IgG | | | | | | | | |
| Surfactant | 53 ± 3 | 0.7 ± 0.2 | 3.1 ± 0.1 | 85 ± 3 | 57 ± 4 | 0.7 ± 0.3 | 3.2 ± 0.1 | 89 ± 3 |
| free nano | | | | | | | | |
| 0.1% tween | 55 ± 4 | 1.4 ± 0.4 | 2.4 ± 0.2 | 89 ± 3 | 53 ± 4 | 1.9 ± 0.3 | 2.2 ± 0.3 | 86 ± 2 |
| 80 nano | | | | | | | | |
| 0.1% tween | 68 ± 3 | 0.8 ± 0.2 | 2.7 ± 0.3 | 91 ± 2 | 66 ± 2 | 0.7 ± 0.1 | 3.1 ± 0.2 | 90 ± 4 |
| 20 nano | | | | | | | | |
| 0.1% brij | 67 ± 4 | 0.8 ± 0.3 | 20 ± 0.2 | 89 ± 3 | 65 ± 4 | 0.7 ± 0.3 | 2.4 ± 0.2 | 89 ± 1 |
| 97 nano | | | | | | | | |

^{a}Fine particle fraction
^{b}Mass median aerodynamic diameter fiom cascade impaction
^{c}Geometric standard deviation
^{d}Emitted dose

TABLE 3
SE-HPLC analysis of reconstituted IgG nanoparticles
produced from 7.5 mg/ml solutions.

| | % | % | % | % | % Relative |
|---|---|---|---|---|---|
| Sample | Monomer* | dimer* | HMW* | Fragment* | recovery^{a} |
| Unprocessed IgG | 98.2 ± 0.1 | 0.9 ± 0.1 | 0.9 ± 0.1 | 0.0 | 100 ± 0.0 |
| Particles precipitated | 72.6 ± 0.2 | 8.4 ± 0.3 | 6.1 ± 0.2 | 14.3 ± 0.2 | 89.2 ± 0.4 |
| without surfactant | | | | | |
| (negative control) | | | | | |
| Particles precipitated | 98.4 ± 0.2 | 0.6 ± 0.2 | 0.8 ± 0.2 | 0.2 ± 0.1 | 96.6 ± 0.2 |
| with 0.1% Tween 80 | | | | | |
| Particles precipitated | 96.2 ± 0.3 | 1.2 ± 0.3 | 1.8 ± 0.4 | 0.7 ± 0.2 | 97.1 ± 0.2 |
| with 0.2% Tween 80 | | | | | |
| Particles precipitated | 96.8 ± 0.1 | 1.1 ± 0.2 | 1.7 ± 0.3 | 0.9 ± 0.3 | 96.8 ± 0.3 |
| with 0.3% Tween 80 | | | | | |
| Particles precipitated | 97.3 ± 0.3 | 0.9 ± 0.2 | 1.1 ± 0.2 | 0.7 ± 0.2 | 98.2 ± 0.2 |
| with 0.1% Tween 20 | | | | | |
| Particles precipitated | 96.9 ± 0.2 | 1.0 ± 0.3 | 1.7 ± 0.4 | 0.7 ± 0.3 | 98.2 ± 0.3 |
| with 0.2% Tween 20 | | | | | |
| Particles precipitated | 97.4 ± 0.2 | 0.8 ± 0.1 | 0.9 ± 0.4 | 0.9 ± 0.2 | 98.8 ± 0.4 |
| with 0.3% Tween 20 | | | | | |
| Particles precipitated | 98.2 ± 0.1 | 0.9 ± 0.2 | 0.6 ± 0.2 | 0.6 ± 0.2 | 97.3 ± 0.3 |
| with 0.1% Brij 97 | | | | | |
| Particles precipitated | 96.6 ± 0.3 | 1.1 ± 0.2 | 1.6 ± 0.2 | 0.2 ± 0.3 | 98.1 ± 0.2 |
| with 0.2% Brij 97 | | | | | |
| Particles precipitated | 97.2 ± 0.1 | 1.0 ± 0.2 | 0.9 ± 0.1 | 0.8 ± 0.2 | 97.6 ± 0.2 |
| with 0.3% Brij 97 | | | | | |

*Data presented as Mean ± SD, n = 3
^{a}Relative recovery = area under the curve for dissolved nanoparticle/area under the curve for the unprocessed.

TABLE 4
SE-HPLC analysis of reconstituted IgG nanoparticles produced from 10 mg/ml solutions

| | % | % | % | % | % Relative |
|---|---|---|---|---|---|
| Sample | Monomer* | dimer* | HMW* | Fragment* | recovery^{a} |
| Unprocessed IgG | 98.2 ± 0.1 | 0.9 ± 0.1 | 0.9 ± 0.1 | 0.0 | 100 ± 0.0 |
| Particles precipitated | 63.3 ± 0.2 | 14.9 ± 0.2 | 10.2 ± 0.3 | 12.2 ± 0.2 | 86.1 ± 0.3 |
| without surfactant | | | | | |
| (negative control) | | | | | |
| Particles precipitated | 92.4 ± 0.3 | 4.6 ± 0.3 | 3.2 ± 0.2 | 0.2 ± 0.2 | 93.3 ± 0.3 |
| with 0.1% Tween 80 | | | | | |
| Particles precipitated | 93.2 ± 0.3 | 4.2 ± 0.1 | 3.6 ± 0.3 | 0.2 ± 0.1 | 94.3 ± 0.2 |
| with 0.2% Tween 80 | | | | | |
| Particles precipitated | 93.1 ± 0.2 | 4.0 ± 0.3 | 3.8 ± 0.2 | 0.1 ± 0.3 | 93.7 ± 0.3 |
| with 0.3% Tween 80 | | | | | |
| Particles precipitated | 92.8 ± 0.3 | 3.4 ± 0.2 | 3.0 ± 0.4 | 0.4 ± 0.2 | 94.2 ± 0.4 |
| with 0.1% Tween 20 | | | | | |
| Particles precipitated | 92.3 ± 0.2 | 3.0 ± 0.3 | 3.7 ± 0.3 | 0.5 ± 0.2 | 95.1 ± 0.1 |
| with 0.2% Tween 20 | | | | | |
| Particles precipitated | 92.1 ± 0.3 | 3.2 ± 0.2 | 3.6 ± 0.2 | 0.3 ± 0.2 | 94.2 ± 0.2 |
| with 0.3% Tween 20 | | | | | |
| Particles precipitated | 93.2 ± 0.2 | 4.0 ± 0.3 | 3.2 ± 0.2 | 0.3 ± 0.1 | 93.1 ± 0.3 |
| with 0.1% Brij 97 | | | | | |
| Particles precipitated | 92.1 ± 0.3 | 3.1 ± 0.2 | 3.9 ± 0.2 | 0.7 ± 0.1 | 92.8 ± 0.3 |
| with 0.2% Brij 97 | | | | | |
| Particles precipitated | 91.8 ± 0.2 | 3.9 ± 0.2 | 4.0 ± 0.3 | 0.6 ± 0.3 | 93.1 ± 0.4 |
| with 0.3% Brij 97 | | | | | |

*Data presented as Mean ± SD, n = 3
^{a}Relative recovery = area under the curve for dissolved nanoparticle/area under the curve for the unprocessed.

### Far-UV Circular Dichroism Spectroscopy

Information about the secondary structure of proteins can be obtained in the far-UV region (190-260 nm), which corresponds to the peptide bond absorption (Shoyele et 5 al., 2011, AAPS PharmSciTech. 12:304-311; Hawe et al., 2009, Eur. J. Pharm. Sci. 38:79-87). The far-UV region of the CD spectrum has been explored in this study in order to determine the effect of the surfactants on the secondary structure of the IgG. Further, the effects of the non-ionic surfactants: Tween 80, Tween 20 and Brij 97 on the retention of the secondary structure of the IgG following the nanoprecipitation process were also investigated. FIG. 10 illustrates the finding that the far-UV CD spectrum of the unprocessed IgG was characterized by a negative maximum at a wavelength of 218 nm and a positive maximum at 202 nm. These are characteristics typical for IgG because of their high β-sheet content (Hawe et al., 2009, Eur. J. Pharm. Sci. 38:79-87; Doi & Jirgensons, 1970, Biochemistry 9:1066-1072; Pelton & McClean, 2000, Anal. Biochem. 277:167-176). All the precipitated nanoparticles retained a beta-sheet structure similar to the unprocessed IgG as seen in FIG. 10.

Table 5 illustrates the secondary structural contents of IgG nanoparticles generated from 5, 7.5 and 10 mg/ml IgG solutions. Data in Table 5 indicated that the unprocessed IgG contained approximately 41% beta sheet and 8% alpha helix secondary structures. Following nanoprecipitation, most of the nanoparticles precipitated from 5 mg/ml solutions retained similar secondary structure composition to that of the unprocessed IgG. However, major perturbations were found in nanoparticles precipitated from 7.5 and 10 mg/ml IgG solutions. These results are consistent with the results obtained from SEHPLC analysis and binding activity assay in which nanoparticles precipitated from 7.5 and 10 mg/ml IgG solutions generally contained lesser percentage of monomers and higher binding affinities in comparison to corresponding particles precipitated from both 5 mg/ml.

TABLE 5
Estimated secondary structure contents of IgG nanoparticles as measured by CD

| | % | % | % | % | % Relative |
|---|---|---|---|---|---|
| Sample | Monomer* | dimer* | HMW* | Fragment* | recovery^{a} |
| Unprocessed IgG | 98.2 ± 0.1 | 0.9 ± 0.1 | 0.9 ± 0.1 | 0.0 | 100 ± 0.0 |
| Particles precipitated | 61.2 ± 0.2 | 15.8 ± 0.3 | 11.8 ± 0.3 | 13.5 ± 0.2 | 80.2 ± 0.2 |
| without surfactant | | | | | |
| (negative control) | | | | | |
| Particles precipitated | 89.6 ± 0.2 | 5.8 ± 0.3 | 3.1 ± 0.2 | 1.1 ± 0.2 | 88.3 ± 03 |
| with 0.1% Tween 80 | | | | | |
| Particles precipitated | 90.2 ± 0.1 | 4.2 ± 0.3 | 4.2 ± 0.3 | 1.2 ± 0.1 | 89.1 ± 0.2 |
| with 0.2% Tween 80 | | | | | |
| Particles precipitated | 91.3 ± 0.3 | 4.3 ± 0.2 | 4.4 ± 0.3 | 0.2 ± 0.2 | 88.1 ± 0.3 |
| with 0.3% Tween 80 | | | | | |
| Particles precipitated | 89.3 ± 0.2 | 5.7 ± 0.3 | 4.2 ± 0.2 | 0.9 ± 0.3 | 89.2 ± 0.2 |
| with 0.1% Tween 20 | | | | | |
| Particles precipitated | 90.8 ± 0.3 | 4.2 ± 0.2 | 4.1 ± 0.3 | 0.7 ± 0.2 | 90.1 ± 0.3 |
| with 0.2% Tween 20 | | | | | |
| Particles precipitated | 90.2 ± 0.3 | 4.1 ± 0.3 | 4.4 ± 0.2 | 0.5 ± 0.2 | 91.8 ± 0.2 |
| with 0.3% Tween 20 | | | | | |
| Panicles precipitated | 88.3 ± 0.2 | 5.8 ± 0.2 | 5.5 ± 0.1 | 0.2 ± 0.2 | 90.1 ± 0.3 |
| with 0.1% Brij 97 | | | | | |
| Panicles precipitated | 89.1 ± 0.3 | 5.9 ± 0.3 | 5.4 ± 0.3 | 0.4 ± 0.3 | 92.1 ± 0.2 |
| with 0.2% Brij 97 | | | | | |
| Particies precipitated | 89.2 ± 0.2 | 5.7 ± 0.1 | 5.6 ± 0.2 | 0.2 ± 0.3 | 91.9 ± 0.3 |
| with 0.3% Brij 97 | | | | | |

*Data presented as Mean ± SD, n = 3
^{a}Relative recovery = area under the curve for dissolved nanoparticle/area under the curve for the unprocessed.

TABLE 6A
Summary of the Aerodynamic Particle Size Distribution Metrics for IgG
nanoparticles using Andersen Cascade Impactor (n = 3, Mean ± SD)

| | 5 mg/ml IgG solution | | 7.5 mg/ml IgG solution | | 10 mg/ml IgG solution | |
|---|---|---|---|---|---|---|
| | % Alpha | % Beta | % Alpha | % Beta | % Alpha | % Beta |
| Sample | helix | sheet | helix | Sheet | helix | sheet |
| Unprocessed IgG | 7.7 ± 0.2 | 41.3 ± 9.5 | 7.7 ± 0.2 | 41.3 ± 9.5 | 7.7 ± 0.2 | 41.3 ± 9.5 |
| Particles precipitated with | 7.6 ± 2.9 | 41.2 ± 5.4 | 3.7 ± 0.0 | 44.5 ± 0.0 | 8.4 ± 0.2 | 35.3 ± 1.9 |
| 0.1% Tween 80 | | | | | | |
| Particles precipitated with | 7.7 ± 2.1 | 42.3 ± 0.1 | 11.8 ± 1.5 | 43.9 ± 1.3 | 8.2 ± 0.2 | 23.6 ± 2.5 |
| 0.2% Tween 80 | | | | | | |
| Particles precipitated with | 7.7 ± 1.4 | 42.2 ± 0.1 | 2.8 ± 0.0 | 51.1 ± 0.0 | 8.1 ± 0.1 | 23.4 ± 1.1 |
| 0.3% Tween 80 | | | | | | |
| Particles precipitated with | 7.4 ± 4.8 | 41.5 ± 2.0 | 3.6 ± 0.1 | 45.4 ± 1.5 | 8.16 ± 0.0 | 24.1 ± 0.1 |
| 0.1% Tween 20 | | | | | | |
| Particles precipitated with | 7.0 ± 0.6 | 41.9 ± 2.3 | 9.6 ± 1.4 | 26.8 ± 4.0 | 8.0 ± 0.1 | 22.1 ± 0.1 |
| 0.2% Tween 20 | | | | | | |
| Particles precipitated with | 4.2 ± 0.0 | 45.5 ± 2.7 | 9.6 ± 1.3 | 29.8 ± 7.8 | 13.8 ± 2.9 | 27.3 ± 1.5 |
| 0.3% Tween 20 | | | | | | |
| Particles precipitated with | 7.2 ± 2.0 | 41.1 ± 3.1 | 10.8 ± 0.8 | 38.7 ± 2.3 | 10.5 ± 0.1 | 29.1 ± 0.1 |
| 0.1% Brij 97 | | | | | | |
| Particles precipitated with | 7.8 ± 1.4 | 44 5 ± 4.0 | 9.7 ± 1.3 | 25.6 ± 1.4 | 8.02 ± 0.1 | 22.1 ± 0.1 |
| 0.2% Brij 97 | | | | | | |
| Particles precipitated with | 7.3 ± 3.8 | 41.9 ± 3.5 | 9.8 ± 1.4 | 25.6 ± 1.4 | 8.1 ± 0.1 | 23.4 ± 1.1 |
| 0.3% Brij 97 | | | | | | |
| Particles precipitated | 10.5 ± 0.0 | 29.1 ± 0.0 | 8.6 ± 0.1 | 25.1 ± 1.5 | 8.02 ± 0.0 | 22.1 ± 0.0 |
| without surfactant | | | | | | |
| (negative control) | | | | | | |

TABLE 6B
Summary of Aerodynamic Size Distribution Metrics for IgG1 nanoparticles
using the ACI (n = 3, Mean ± SD)

| | Handihaler | | | | Spinhaler | | | |
|---|---|---|---|---|---|---|---|---|
| | FPF^{a} | MMAD^{b} | | ED^{d} | FPF | MMAD | | ED |
| Particles | (%) | (μm) | GSD^{c} | (%) | (%) | (μm) | GSD | (%) |
| Unprocessed | 20 ± 4 | 8.1 ± 0.4 | 3.2 ± 0.2 | 97 ± 2 | 22 ± 2 | 8.5 ± 0.3 | 2.2 ± 0.3 | 95 ± 3 |
| IgG | | | | | | | | |
| Surfactant free | 53 ± 3 | 0.7 ± 0.2 | 3.1 ± 0.1 | 85 ± 3 | 57 ± 4 | 0.7 ± 0.3 | 3.2 ± 0.1 | 89 ± 3 |
| IgG nano | | | | | | | | |
| 0.1% tween | 55 ± 4 | 1.4 ± 0.4 | 2.4 ± 0.2 | 89 ± 3 | 53 ± 4 | 1.9 ± 0.3 | 2.2 ± 0.3 | 86 ± 2 |
| 80-IgG nano | | | | | | | | |
| 0.1% tween | 68 ± 3 | 0.8 ± 0.2 | 2.7 ± 0.3 | 91 ± 2 | 66 ± 2 | 0.7 ± 0.1 | 3.1 ± 0.2 | 90 ± 4 |
| 20-IgG nano | | | | | | | | |
| 0.1% brij 97- | 67 ± 4 | 0.8 ± 0.3 | 2.0 ± 0.2 | 89 ± 3 | 65 ± 4 | 0.7 ± 0.3 | 2.4 ± 0.2 | 89 ± 1 |
| IgG nano | | | | | | | | |

^{a}Fine particle fraction
^{b}Mass median aerodynamic diameter from cascade impaction
^{c}Geometric standard deviation
^{d}Emitted dose

### Aerosol Performance Test Using ACI

Nanoparticles generated from 5 mg/ml IgG solutions with or without 0.1% surfactant were compared to the unprocessed IgG particles in terms of aerosol performance. Tables 6A-6B illustrates the aerodynamic particle size metrics using both Handihaler® and Spinhaler® DPI devices. The FPF (<4.7 μm) of the unprocessed IgG particles was approximately 20% using handihaler while it was approximately 22% using spinhaler. However, the surfactant free nanoparticles had FPF of 53% using handihaler. The FPF of these nanoparticles using spinhaler (57%) was similar to that of the handihaler, suggesting that the type of device used did not affect the aerosol performance of the nanoparticles. The SEM image of these surfactant-free nanoparticles in FIG. 2 indicated that they are predominantly spherical in shape. The particle size as measured by DLS was 515.7 nm while the MMAD as measured by ACI was approximately 700 nm (0.7 μm) for both devices. Nanoparticles precipitated in the presence of 0.1% Tween 80 having similar spherical shape as the surfactant-free nanoparticles exhibited FPF of 55% using handihaler and FPF of 53% using spinhaler. The MMAD of the nanoparticle precipitated from 0.1 Tween 80 was 1.4 μm using handihaler while it was 1.9 μm using the spinhaler. Nanoparticles precipitated in the presence of 0.1% Tween 80 and Brij 97 exhibited FPF of 68% and 67% respectively using handihaler and 66% and 65% respectively using the spinhaler. The MMAD of these particles were found to be similar as well, with both having MMADs of 0.8 μm using the handihaler and 0.7 μm using the spinhaler. Generally, there were similarities in the aerosol performance of the nanoparticles irrespective of the DPI device used. However, differences were found in the aerosol performance based on the type of nanoparticles.

### Example 2

Results presented above suggest that the nanoprecipitation process was able to produce uncoated IgG nanoparticles by varying the concentration of the IgG in the precipitating solution as well as the type and concentration of the non-ionic surfactants. In this study, Tween 80, Tween 20 and Brij 97 were used for size and shape control during the nanoprecipitation process. They were also applied as protein stabilizing agents to help protect the IgG against the relatively harsh precipitating environment. The concentrations of the surfactants used were many folds higher than their respective critical micelle concentrations (CMC) in water which are 0.0017%, 0.0075% and 0.029% for Tween 80, Tween 20 and Brij 97 respectively.

Data from DLS (Tables 1) illustrated that the size of the particles produced by this technology was in size range 90-800 nm. Generally, a surfactant concentration-dependent increase in particle size was observed for all the nanoparticles. Further, there was a general decrease in the size of the nanoparticles as the concentration of the IgG in the precipitating medium increased from 5 mg/ml to 10 mg/ml. For instance, particles precipitated from 5 mg/ml IgG solution in the presence of 0.2% Tween 80 were 220.7 nm, while particles precipitated from 7.5 mg/ml IgG solution in the presence of same concentration of Tween 80 (0.2%) was 151.8 nm. Increasing the concentration of IgG to 10 mg/ml further decreased the size of the particles to 131.9 nm. Without wishing to be limited by theory, the surfactant concentration-dependent increase in particle size exhibited by the nanoparticles could be attributed to an increasing influence of the surfactant in the self-association of the IgG molecules during the precipitation process. Without wishing to be limited by theory, it is possible that, because surfactants have a natural tendency to self-assemble into micelles at concentrations above the CMC, they probably aid the self-association of the IgG by including the IgG in the self-assembly process, since the surfactants exhibit hydrophobic interaction with the IgG.

SEM micrographs (FIGS. 1-5) illustrated the finding that the morphology (shape) of the particles produced by the nanoprecipitation technology may be influenced by the type of surfactant incorporated in the precipitating solution. By changing the type of surfactant (Tween 80, Tween 20 or Brij 97) and keeping all other conditions constant, i.e., concentration of IgG solution (5 mg/ml) and concentration of surfactant (0.1% w/v), nanoparticles with different shapes were produced. Nanoparticles precipitated in the presence of 0.1% w/v were predominantly spherical in shape (FIG. 3). However, when Tween 80 was replaced with Tween 20, the particles generated were polyhedral in shape while the nanoparticles precipitated in the presence of Brij 97 were sponge-like in shape. This suggested that an adequate control of particle shape could be achieved by varying the type of surfactant in the precipitating medium. The change in particle shape may be attributed to the differences in the molecular structures of the surfactants used. It has been previously reported that the self-assembly pattern of nanostructures are influenced by the molecular structure of surfactants applied in the fabrication process, although the mechanisms through which this occurs is not yet fully understood (Tummala & Alberto, 2009, ACS Nano. 3:595-602; Suttipon et al., 2011, J. Phys. Chem. C. 115:17286-17296). Interestingly, the particle size data as provided by DLS seem marginally different from the size of the particles visually observed from the SEM micrographs. This is due to the fact that the DLS measures the hydrodynamic diameter due to the solvent effect (Liv et al., 2005, J. Agric. Food Chem. 53:437-441), while SEM only gives an estimate of the particle size and is not always accurate. Further, many biological compounds (e.g., liposomes and proteins) and engineered polymers (e.g., dendrimers) are invisible to SEM without heavy-metal staining procedures because these compounds do not deflect an electron beam sufficiently. For these ‘soft’ particles, SEM does not always provide a relevant measure of particle size (Hall et al., 2007, Future Medicine-Namomedicine 2(6):789-803).

The conformational structure of the IgG would be expected to be maintained after the reconstitution of the nanoparticles in a buffer (if needed), or after going into dissolution in the body. It is also expected that these nanoparticles would go into dissolution at an acidic pH due to its isoelectric point (pI). It is well established that major perturbations in the conformational structure of a protein often lead to a reduction or even a loss of the biological activity of the protein (Shoyele & Slowey, 2006, Intl. J. Pharm. 314:1-8, Hawe et al., 2009, Eur. J. Pharm. Sci. 38:79-87, Sviridov et al., 1988, Biokhimiia. 53:61-68; Manning et al., 1989, Pharm. Res. 6:903-18). Further, soluble aggregates in protein formulations are undesirable because of their immunogenicity (Rosenberg, 2006, The AAPS Journal. 8(3): Article 59; Cleland et al., 1993, Crit. Rev. Ther. Drug Carrier Syst. 10:307-377; Hermeling et al., 2003, Pharm Res. 20:1903-1907). SE-HPLC was used to investigate the presence of soluble aggregates as well as fragments in the reconstituted IgG nanoparticles. The nanoparticles were reconstituted at pH 5 and not 7, since proteins have minimum solubility at their isoelectric point (pI) (Ingham, 1990, Precipitation of proteins with polyethylene glycol. In: Deutscher (Ed): Guide to protein purification. San Diego: Academic Press. pp. 301-306; Ries-Kautt & Ducruix, 1992, Phase diagrams. In: Ducruix A., Giege, R (Ed). Crystallization of nucleic acids and proteins. 1^{St} edition. Oxford: Oxford University Press, pp. 195-218). Further, should there be a need for the nanoparticles to be reconstituted before application, slightly acidic buffers would be the solvents of choice due to the pI of the antibody. It is therefore important that the IgG is stable at this pH following reconstitution. As illustrated in FIG. 9, the unprocessed IgG (positive control) showed a prominent peak at approximately 16 minutes indicating that the unprocessed IgG predominantly contained monomeric IgG. Using the ratio of the total peak area of the respective nanoparticles to that of the unprocessed IgG, percentage recovery was determined for each reconstituted nanoparticles as illustrated in Tables 2-4.

Nanoparticles precipitated form 10 mg/ml solutions generally had lower monomer contents in comparison to the nanoparticles precipitated from corresponding 5 mg/ml and 7.5 mg/ml solutions. Without wishing to be limited by theory, this could be attributed to the relatively higher concentration of IgG in the precipitating solution. Increased concentration-induced aggregation probably occurs because of increased intermolecular proximity of the IgG molecules. Due to the increased proximity, partially unfolded IgG molecules would have increased tendency to aggregate.

The decrease in the amount of monomers recovered from the nanoparticles precipitated from the surfactant-free IgG solutions was not only due to aggregation but also due to fragment formation during the nanoprecipitation process. The surfactant-free IgG particles precipitated from 5 mg/ml solution had 14% fragment (Table 2) in comparison to nanoparticles precipitated in the presence of surfactants having <1% fragments. Fragments formation may be attributed to the hydrolysis of the peptide bonds by the HCl that was used to precipitate the nanoparticles (Schrier et al., 1993, Pharm Res. 10:933-944; Kenley & Warne, 1994, Pharm Res. 11:72-76; Bond et al., 2010, J. Pharm. Sci. 99:2582-2597). Following the addition of the non-ionic surfactant to the precipitating solution, the nanoparticles formed were more stable (Tables 2-4) in comparison to the particles precipitated from the surfactant-free solution. These results suggest that the non-ionic surfactants included in the precipitating solutions may help to stabilize the IgG during the precipitation process. The stabilization may be attributed to the hydrophobic interaction with protein molecules (Bam et al., 1998, J. Pharm. Sci. 87:1554-9; Tandon & Horowitz, 1987, J. Biol. Chem. 262:4486-91 41). It is also possible that the surfactants form micelles around the IgG molecules during the precipitation process, thereby protecting them from the harsh precipitating environment.

Binding activity data presented in FIGS. 6-9 are consistent with the SE-HPLC data. FIG. 6 illustrates the finding that Tween 80 seems to improve the bioactivity of the nanoparticles in comparison to the nanoparticles precipitated from the surfactant free IgG solutions. Further, nanoparticles from 5 mg/ml solutions had superior binding activity to those precipitated from corresponding 7.5 and 10 mg/ml solutions. For instance, nanoparticles precipitated from 5 mg/ml solution in the presence of 0.1% Tween 80 retained approximately 109% bioactivity while corresponding nanoparticles from 7.5 mg/ml and 10 mg/ml solutions only retained approximately 90% and 80% binding activity respectively. This trend was consistent for the other surfactants as illustrated in FIGS. 7-8. The specific binding activity results suggest that the monomer contents of the reconstituted nanoparticles had a great impact on their binding activity.

Data in Table 5 suggest that the surfactants contributed to the retention of the secondary structure in the IgG nanoparticles. The nanoparticles precipitated from surfactant-free solutions, irrespective of the concentration of the IgG, showed major perturbations in their secondary structures. While the unprocessed IgG contained approximately 40% beta-sheet, the surfactant-free nanoparticles from 5, 7.5 and 10 mg/ml IgG solutions contained approximately 29, 25 and 22% beta sheet respectively. Following the addition of 0.1% Tween 80 to the precipitating solution, the nanoparticles produced from the 5 and 7.5 mg/ml solutions had similar percentages of beta sheet to the unprocessed IgG, which were 41 and 45% respectively. Although the % beta sheet content of the corresponding nanoparticles precipitated from 10 mg/ml IgG solution was slightly lower (35%), this was relatively higher than the negative control (surfactant-free). Generally, nanoparticles precipitated from 5 mg/ml IgG solutions had higher beta-sheet contents in comparison to the corresponding nanoparticles precipitated from both 7.5 and 10 mg/ml solutions. Without wishing to be limited by theory, this may be caused by the higher ratio of IgG to surfactants in these solutions of higher protein concentrations in comparison to 5 mg/ml solution, which obviously had lower ratio of IgG to surfactants in the respective solutions. Due to the relatively lower ratio of IgG to surfactants in the 5 mg/ml, more molecules of IgG were probably better protected by the surfactants than in the corresponding 7.5 mg/ml and 10 mg/ml solutions suggesting that protein concentration is an important variable for ameliorating aggregation in this technology.

Table 4 illustrates the in vitro deposition patterns of IgG nanoparticles as analyzed by ACI using both Handihaler® and Spinhaler® DPI devices. Similar trends were observed for the aerosol deposition patterns of both devices. However, the surfactant-free IgG nanoparticles, IgG nanoparticles precipitated in the presence of 0.1% Tween 80, 0.1% Tween 20 and Brij 97 appear to have superior aerosol performance in comparison to the unprocessed IgG. Comparing the SEM micrographs of the nanoparticles (FIGS. 1-5) to their respective aerosol performance allows to assess the impact of particle shape on aerosol performance of the nanoparticles. For instance, nanoparticles precipitated in the presence of 0.1% Tween 80 were polyhedral in shape while nanoparticles precipitated in the presence of Brij 97 were sponge-like in shape. These nanoparticles exhibited FPFs of approximately 66% using both DPI devices. However, nanoparticles precipitated in the presence of 0.1% Tween 80 were spherical similar to the surfactant-free nanoparticles. While the surfactant-free nanoparticles exhibited FPF of 53 and 57% in both handihaler and spinhaler respectively, nanoparticles precipitated from 0.1% Tween 80 (with similar spherical shape) had FPF of approximately 55%. Elongated and pollen-shaped particles tend to exhibit better flowability and higher FPF than spherical particles, because elongated particles have longer suspended time in the air and can travel further in the lung airways (Hassan & Lau, 2009, AAPSPharmSciTech. 10:1252-1262). Further, the sponge-like particles exhibited rough surfaces and particles with rough surfaces tend to have improved flowability due to reduced van Der Waal forces because of reduced inter-particulate contact area (Hassan & Lau, 2009, AAPSPharmSciTech. 10:1252-1262). This improved flowability ultimately leads to enhanced aerosol performance (Hassan & Lau, 2009, AAPSPharmSciTech. 10:1252-1262).

The present work has demonstrated a method of producing nanoparticles that could potentially be used for pulmonary delivery of monoclonal antibodies. Stable IgG nanoparticles were produced by using non-ionic surfactants as stabilizers and for shape and size control. Careful control of the shape can improve the aerosol performance of antibody nanoparticles. Elongated and sponge shaped nanoparticles were found to exhibit superior aerosol performance in comparison to spherical nanoparticles.

### Example 3

### Bevacizumab

Bevacizumab is a monoclonal antibody against VEGF, a potent angiogenetic factor. The biological activity of bevacizumab is based on its anti-angiogenetic effect by blocking VEGF.

### Particle Characterization

The percentage yield of bevacizumab nanoparticles as determined by UV absorption at 280 nm was found to vary between 90% and 103%.

The size, zeta potential, and morphology of the self-associated bevacizumab nanoparticles were measured by PCS (Zetasizer) and SEM. Table 7 illustrates the influence of non-ionic surfactants on the particle size and charge density on the surface of self-associated bevacizumab nanoparticles. The surfactants seemed to have a concentration-dependent influence on the size and zeta potential of the nanoparticles. The size of the unprocessed bevacizumab particles could not be measured using photon correlation spectroscopy (PCS) because the instrument can only be used for particles between 0.3 nm and 10 μm. According to the Manufacturer's Manual, PCS is based on Brownian motion and particles with size above 10 μm tend to quickly sediment. In certain embodiments, under these circumstances, size recorded by the instrument may be due to artifact. However, the size of the unprocessed bevacizumab as estimated from the SEM measurement was approximately 20 μm. For surfactant controls, distinct concentrations of the surfactants used in the nanoprecipitation process were dissolved in 0.01 N HCl and titrated with 0.1 N NaOH up to pH 8.4. The zeta potential of the control surfactants was measured, and the result is presented in Table 8. In the case of nanoparticles, an increase in size was observed as the concentration of the non-ionic surfactant in the precipitation medium increased. For instance, bevacizumab nanoparticles precipitated from a precipitation medium containing 0.1%, 0.2%, and 0.3% w/v Tween 80 measured 395.4±2.2, 552.8±0.8, and 668.4±1.1 nm respectively. The same trend was observed with the zeta potential measurement.

Generally, an increase in particle size was observed as the concentration of the surfactant increased. Without wishing to be bound by theory, it may be caused by increased interaction of the surfactants with the protein molecules, since non-ionic surfactants are known to interact with protein through hydrophobic interaction. As a proposed mechanism by which the surfactants affect the size of the nanoparticles, the surfactants may self-assemble above their CMC, and the protein molecules are included in the self-assembling process. The particle size data showed similar trends for both IgG1 and bevacizumab, suggesting that the nanoprecipitation technology could be applied to antibodies in general.

FIGS. 11A-11E illustrate SEM micrographs of bevacizumab particles obtained under different conditions. The SEM micrograph in FIG. 11A revealed that the unprocessed bevacizumab (lyophilized bevacizumab from the supplied solution after dialysis to remove any excipients in the solution) was platelike and irregularly shaped. However, the surfactant-free bevacizumab nanoparticles (FIG. 11B) were spherical but agglomerated. 0.1% Tween 80-containing bevacizumab nanoparticles (FIG. 11C) appeared similar to the nanoparticles containing 0.1% w/v Tween 20 (FIG. 11D). However, 0.1% w/v Brij 97-containing nanoparticles appeared spongelike in shape (FIG. 11E).

TABLE 7

| | 5 mg/ml bevacizumab solution | | |
|---|---|---|---|
| | Particle | | Zeta* |
| | Diameter | | potential |
| Sample | (nm)* | PDI* | (mV) |
| Unprocessed bevacizumab | N/A | N/A | −4.6 ± 0.3 |
| Particles precipitated | 395.4 ± 2.2 | 0.2 ± 0.0 | −13.7 ± 0.7 |
| with 0.1% Tween 80 | | | |
| Particles precipitated | 552.8 ± 0.8 | 0.1 ± 0.0 | −18.1 ± 0.8 |
| with 0.2% Tween 80 | | | |
| Particles precipitated | 668.4 ± 1.1 | 0.2 ± 0.0 | −25.6 ± 1.5 |
| with 0.3% Tween 80 | | | |
| Particles precipitated | 334.4 ± 2.6 | 0.2 ± 0.0 | −10.5 ± 0.5 |
| with 0.1% Tween 20 | | | |
| Particles precipitated | 530.9 ± 5.9 | 0.2 ± 0.0 | −12.0 ± 0.1 |
| with 0.2% Tween 20 | | | |
| Particles precipitated | 634.9 ± 9.5 | 0.2 ± 0.0 | −13.5 ± 0.3 |
| with 0.3% Tween 20 | | | |
| Particles precipitated | 365.6 ± 4.1 | 0.1 ± 0.1 | −9.7 ± 0.4 |
| with 0.1% Brij 97 | | | |
| Particles precipitated | 596.5 ± 1.5 | 0.1 ± 0.0 | −11.7 ± 0.6 |
| with 0.2% Brij 97 | | | |
| Particles precipitated | 774.2 ± 0.9 | 0.1 ± 0.0 | −13.3 ± 0.2 |
| with 0.3% Brij 97 | | | |
| Particles precipitated | 239.2 ± 1.0 | 0.1 ± 0.0 | −5.1 ± 0.7 |
| without surfactant | | | |
| (negative control) | | | |

*Data presented as Mean ± SD.

TABLE 8
Zeta Potential of Surfactant Controls Measured by PCS

| Surfactant control | Zeta potential (mV) |
|---|---|
| 0.1% Tween 80 | −12.9 ± 0.8 |
| 0.2% Tween 80 | −13.1 ± 1.2 |
| 0.3% Tween 80 | −13.4 ± 1.5 |

### Retained Biological Activity

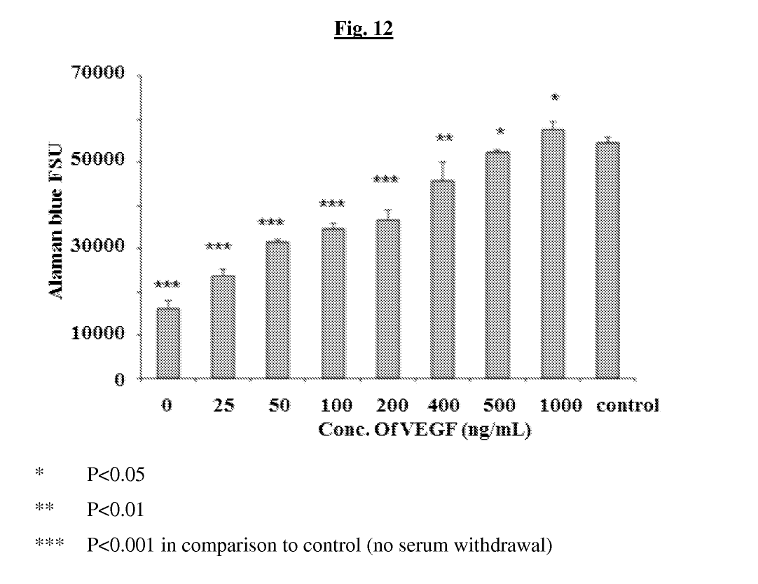

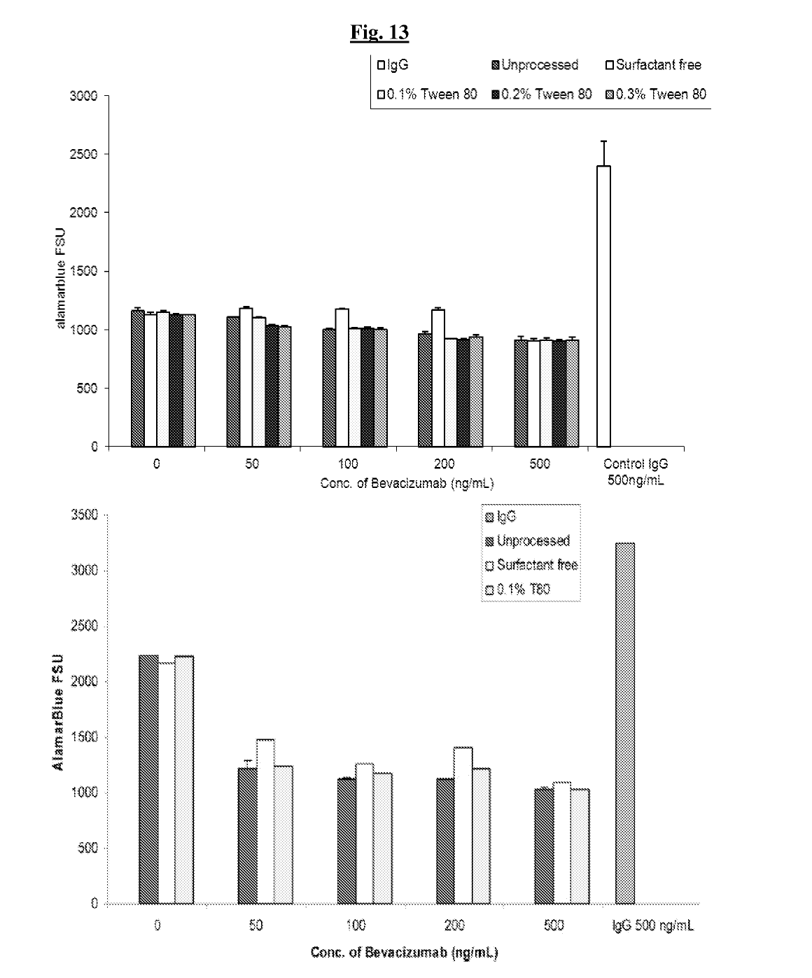

Firstly, to measure proliferation, HUVEC were incubated with various concentrations of rhVEGF (0-1000 ng/ml). This experiment had the objective of showing the importance of VEGF in the growth of HUVEC following serum withdrawal. As control, HUVEC with full growth factors including serum. Data are presented in FIG. 12.

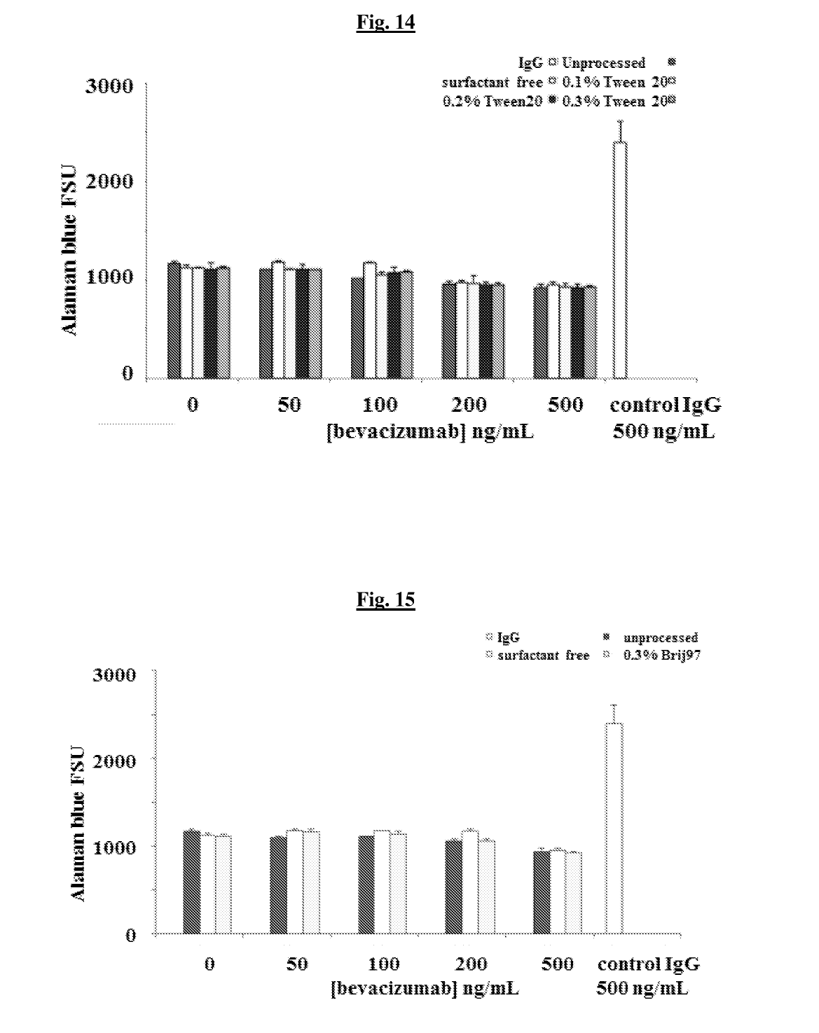

For bevacizumab inhibition of VEGF, various concentrations of bevacizumab were mixed with 50 ng/ml of VEGF at 37° C. for 2 hours before the addition of HUVEC. The plate was then incubated at 37° C. with 5% CO₂ for days. At the end of the incubation, 25 μl of alamar blue was added to each well and the plate was incubated for an additional 6 h under the same conditions. The plate was then read at 530/590 nm on a fluorescence plate reader. Alamar blue dye is a growth indicator based on metabolic activity, which is reflective of the extent of cellular proliferation. IgG1 and dissolved surfactants were used as controls. N=4 for all experiments. Results are summarized in FIGS. 14-16.

The surfactant-free nanoparticles did not show any effect until the maximum concentration of 500 ng/ml, suggesting loss of stability without the surfactant acting as a stabilizer. However, Tween 80 containing nanoparticles showed a concentration dependent anti-proliferation effect. Students T test showed no significant difference (P>0.05) between the activity of the surfactant-containing nanoparticles and the unprocessed bevacizumab. However, surfactant-free nanoparticles were significantly different (P<0.05) from the unprocessed at all concentrations, except 500 ng/ml.

### MIT Assay Using A549 Cell Line

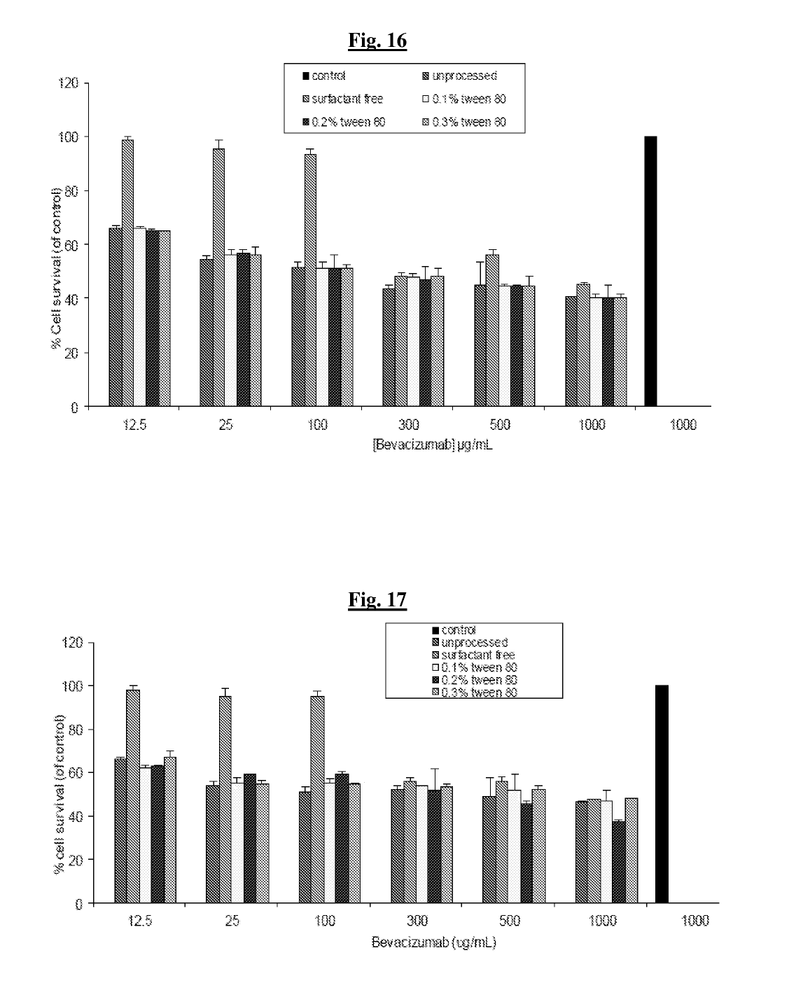

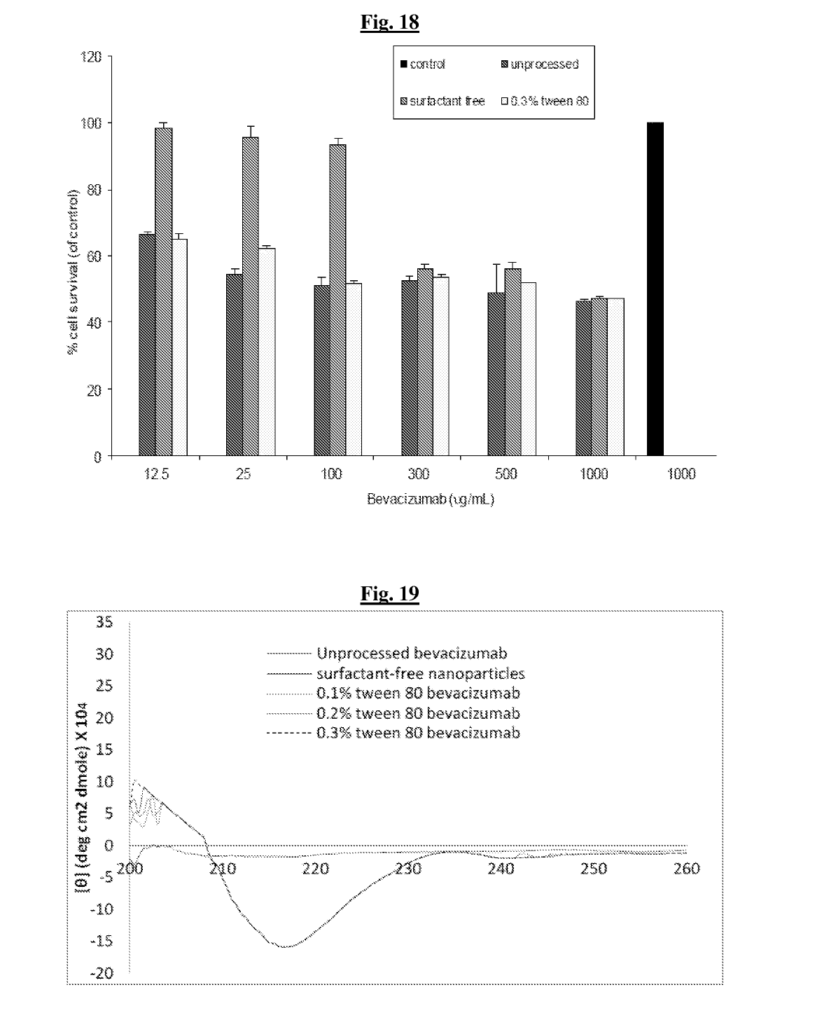

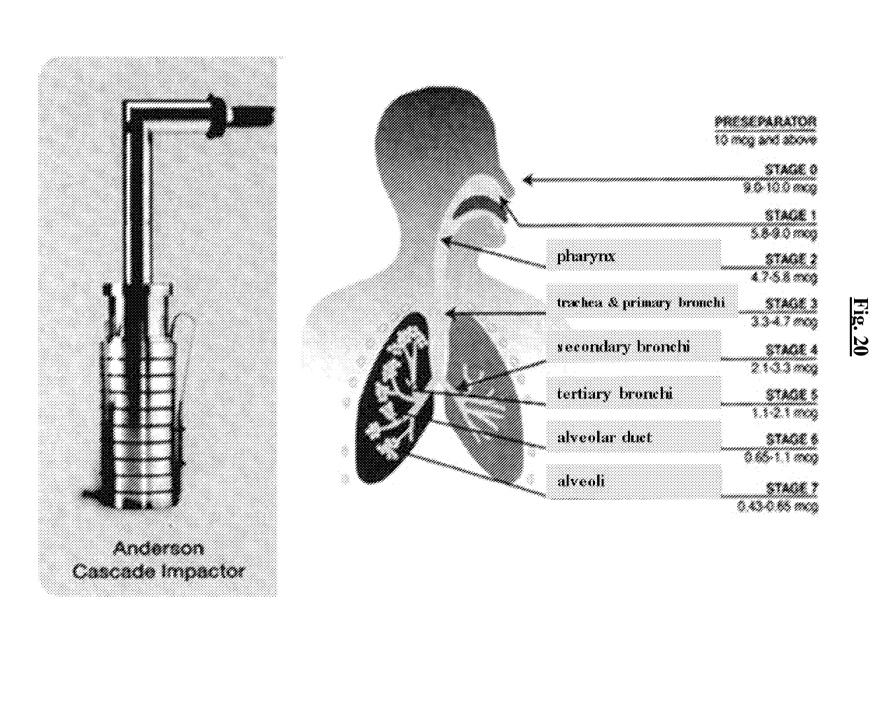

The cytotoxicity of the reconstituted bevacizumab nanoparticles against A549 cell line (a non-small lung cancer cell line) was investigated using MTT assay. The control contained the appropriate concentrations of the relevant surfactant. (FIGS. 16-18). The MTT assay revealed that the reconstituted nanoparticles generally have similar cytotoxicity to that of the unprocessed bevacizumab, except for the surfactant-free nanoparticles that were significantly different (P>0.05) from the unprocessed bavacizumab across concentrations.

The IC₅₀ of the reconstituted Tween 80-containing bevacizumab nanoparticles was similar to that of the unprocessed bevacizumab particles, which was estimated to be approximately 1.8 μM. However, the surfactant-free bevacizumab nanoparticle formulation was not as effective as the other forms, and that produced an IC₅₀ of approximately 3.3 μM. This result correlates well with the data from the anti-VEGF studies using HUVECs. Similar results were obtained for both Tween 20- and Brij 97-containing nanoparticles.

### SE-HPLC

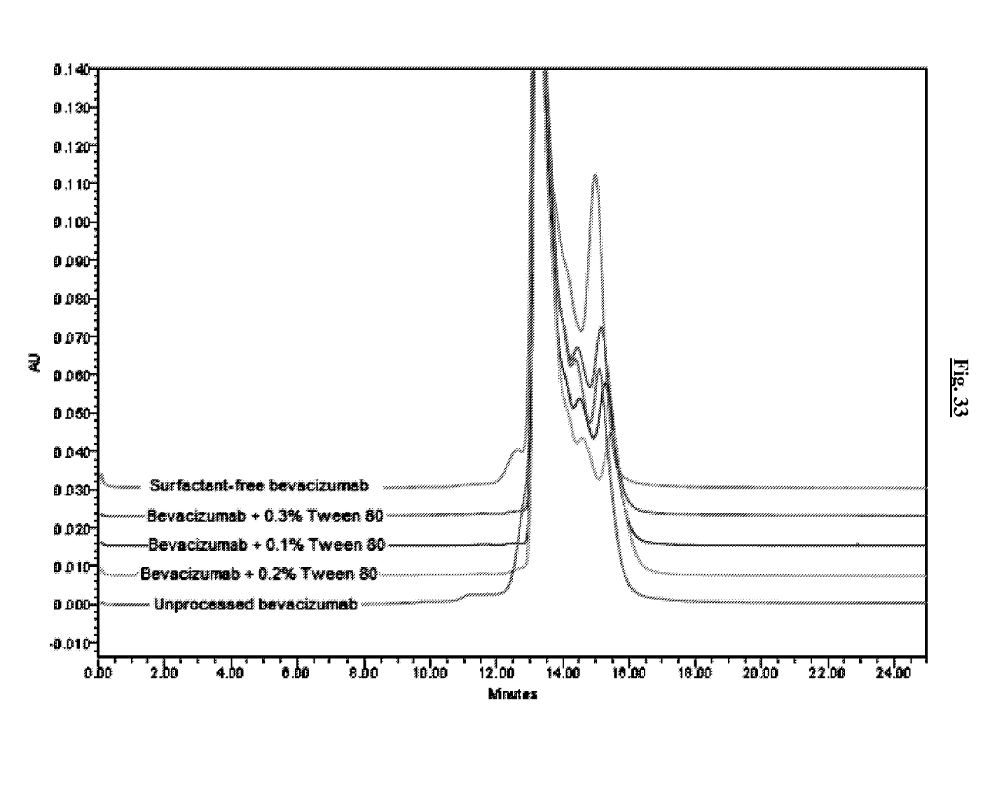

The presence of monomers, soluble aggregates and fragments in the reconstituted antibody nanoparticles was investigated using SE-HPLC (FIG. 33). Soluble aggregates in reconstituted protein formulations are undesirable because of their immunogenicity. Further, the presence of fragments in the reconstituted formulation suggests structural instability in the protein. The nanoparticles were reconstituted at pH 5 prior to SE-HPLC because it is believed that this is the pH that the nanoparticles would go into dissolution in the cellular endosomes (with pH range 4-6).

TABLE 9
SE-HPLC analysis of reconstituted bevacizumab nanoparticles produced
from 5 mg/ml solutions. Control samples showed no discernible peaks.

| | % | % | % | % | % Relative |
|---|---|---|---|---|---|
| Sample | Monomer* | dimer* | HMW* | Fragment* | recovery^{a} |
| Unprocessed bevacizumab | 94.9 ± 0.4 | 0.0 | 0.0 | 5.1 ± 0.1 | 100.0 ± 0.0 |
| Particles precipitated | 94.6 ± 0.2 | 0.0 | 0.0 | 5.5 ± 0.2 | 112.9 ± 2.0 |
| without surfactant | | | | | |
| (negative control) | | | | | |
| Particles precipitated | 93.9 ± 0.1 | 0.0 | 0.0 | 6.0 ± 0.1 | 103.6 ± 1.5 |
| with 0.1% Tween 80 | | | | | |
| Particles precipitated | 85.2 ± 0.4 | 0.8 ± 0.0 | 0.0 | 14.0 ± 0.4 | 110.6 ± 1.9 |
| with 0.2% Tween 80 | | | | | |
| Particles precipitated | 93.6 ± 0.1 | 0.0 | 0.0 | 6.4 ± 0.1 | 98.3 ± 1.4 |
| with 0.3% Tween 80 | | | | | |
| Particles precipitated | 93.7 ± 0.5 | 0.0 | 0.0 | 6.3 ± 0.2 | 107.1 ± 1.5 |
| with 0.1% Tween 20 | | | | | |
| Particles precipitated | 92.7 ± 0.6 | 0.0 | 0.0 | 7.3 ± 0.4 | 106.0 ± 3.8 |
| with 0.2% Tween 20 | | | | | |
| Particles precipitated | 91.9 ± 0.6 | 0.0 | 0.0 | 8.2 ± 0.5 | 105.0 ± 0.5 |
| with 0.3% Tween 20 | | | | | |
| Particles precipitated | 94.8 ± 0.9 | 0.0 | 0.0 | 5.1 ± 1.0 | 116.7 ± 6.1 |
| with 0.1% Brij 97 | | | | | |
| Particles precipitated | 84.1 ± 0.6 | 1.2 ± 0.0 | 0.0 | 15.0 ± 0.3 | 106.3 ± 0.6 |
| with 0.2% Brij 97 | | | | | |
| Particles precipitated | 93.0 ± 1.0 | 0.0 | 0.0 | 7.0 ± 1.0 | 109.1 ± 1.8 |
| with 0.3% Brij 97 | | | | | |

*Data presented as Mean + SD, n = 3
^{a}Relative recovery = area under the curve for dissolved nanoparticle/area under the curve for the unprocessed.

### Secondary Structure Analysis Using Far-UV Circular Dichroism (CD)

Far UV CD was used to probe for perturbations in the secondary structure of the antibodies following reconstitution in acetate buffer (pH 6). This was done to test whether the secondary structure of the antibodies was still intact, as major perturbation in the secondary structure of proteins may lead to loss or reduction of biological activity. Far-UV CD spectra (FIG. 19) suggested that the secondary structure of bevacizumab was mainly β-sheet. Further, the spectra show that the nanoprecipitation process did not lead to any major perturbations in the structure of bevacizumab when the precipitation was done in the presence of the non-ionic surfactants. However, major perturbations were observed in the secondary structure of the surfactant-free bevacizumab nanoparticles following reconstitution. These perturbations in the surfactant-free bevacizumab may be responsible for the reduced activity observed for this formulation in the bioactivity study. To further investigate the perturbations in the secondary structure, K2D2 software was used to estimate the secondary structure of the bevacizumab nanoparticles using data obtained from the far-UV analysis.

TABLE 10

| Sample | % alpha helix | % β-sheet |
|---|---|---|
| Avastin unprocessed | 7.52 | 32.19 |
| Surfactant-free | 1.82 | 47.71 |
| 0.1% T80 | 7.86 | 32.03 |
| 0.2% T80 | 7.82 | 32.07 |
| 0.3% T80 | 7.82 | 32.07 |
| 0.1% T20 | 7.82 | 32.07 |
| 0.2% T20 | 7.52 | 33.12 |
| 0.3% T20 | 7.44 | 33.12 |
| 0.1% B97 | 7.44 | 32.28 |
| 0.2% B97 | 7.82 | 32.01 |
| 0.3% B97 | 7.82 | 32.01 |

### Cellular Internalization Studies

Using non-small lung cancer cell line A459, the internalization behavior of these nanoparticles was accessed and compared to that of the unprocessed bevacizumab particles. The internalization studies were performed using techniques such as fluorescence, microscopy, TEM and flow cytometry to address two questions: (1) whether the particles were internalized by these cells despite their negative charges in comparison to the unprocessed particles, (2) to gain a preliminary insight into the mechanisms of internalization.

### Indirect Labeling of Nanoparticles

The cells were grown to 80% confluence and then incubated with 0.1% Tween 80: bevacizumab (as a proof of concept) for 6 minutes. The cells were then thoroughly washed. To label the nanoparticles inside the cell, the cells were treated with anti-human IgG monoclonal antibody conjugated to FITC following permeabilization with saponin. DAPI was used for nuclear staining while wheat germ agglutinin conjugated to Alexa-fluor-55 was used to stain the cell membrane.

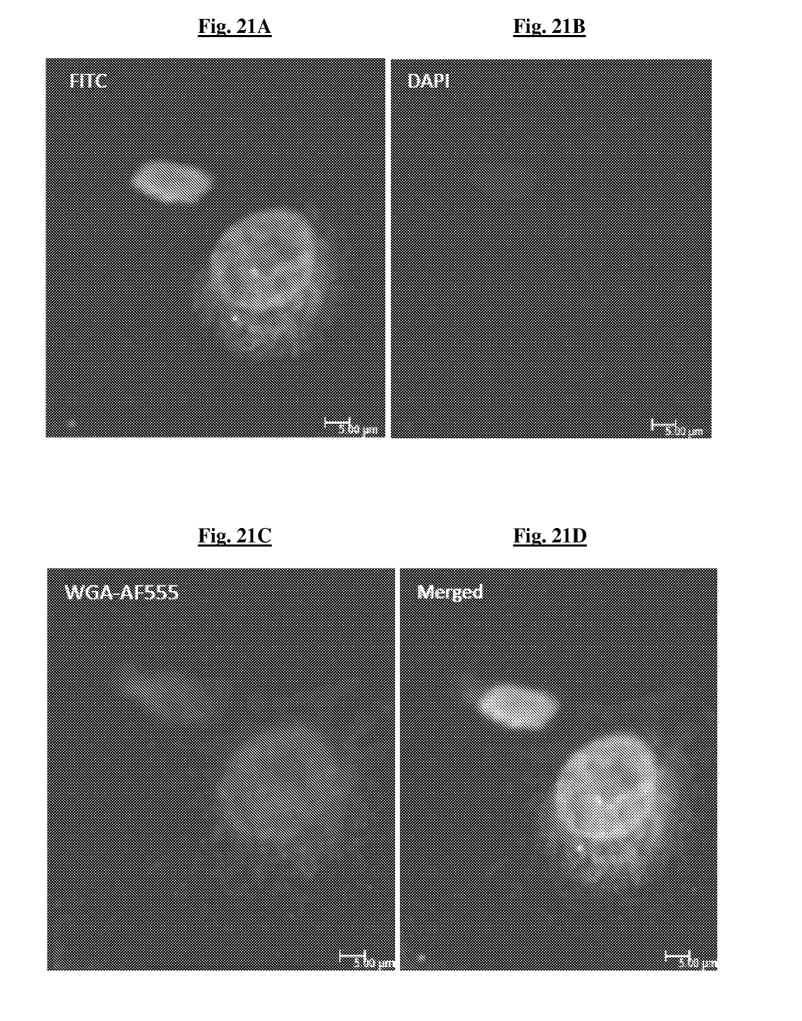

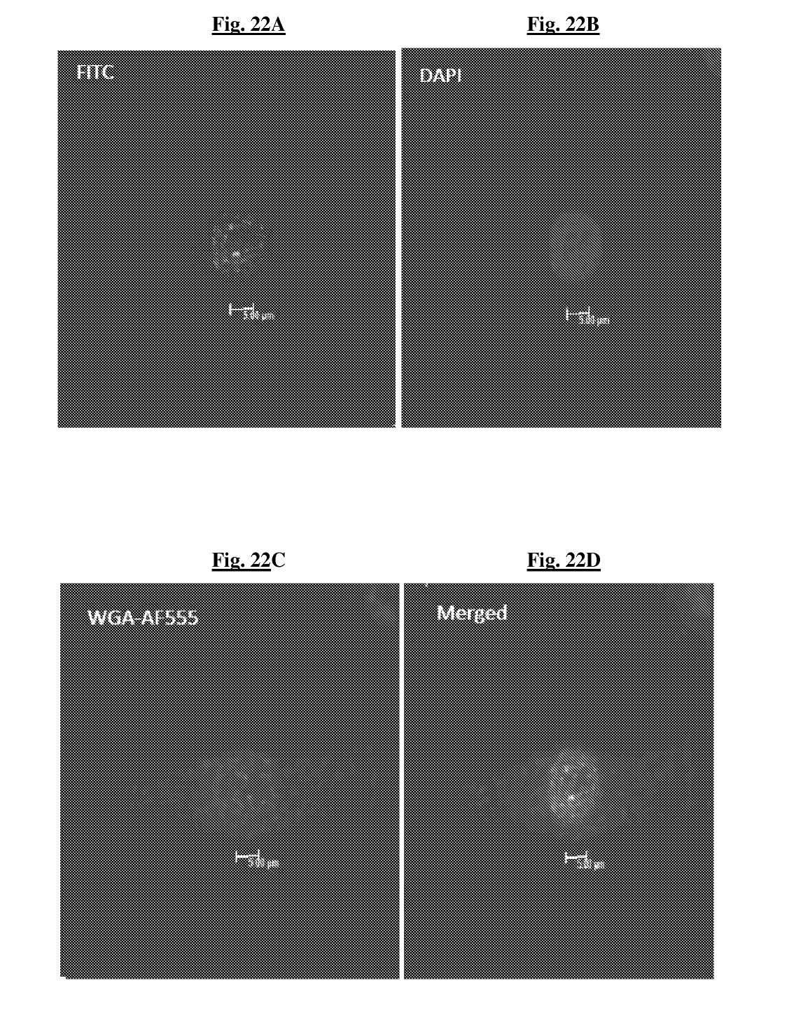

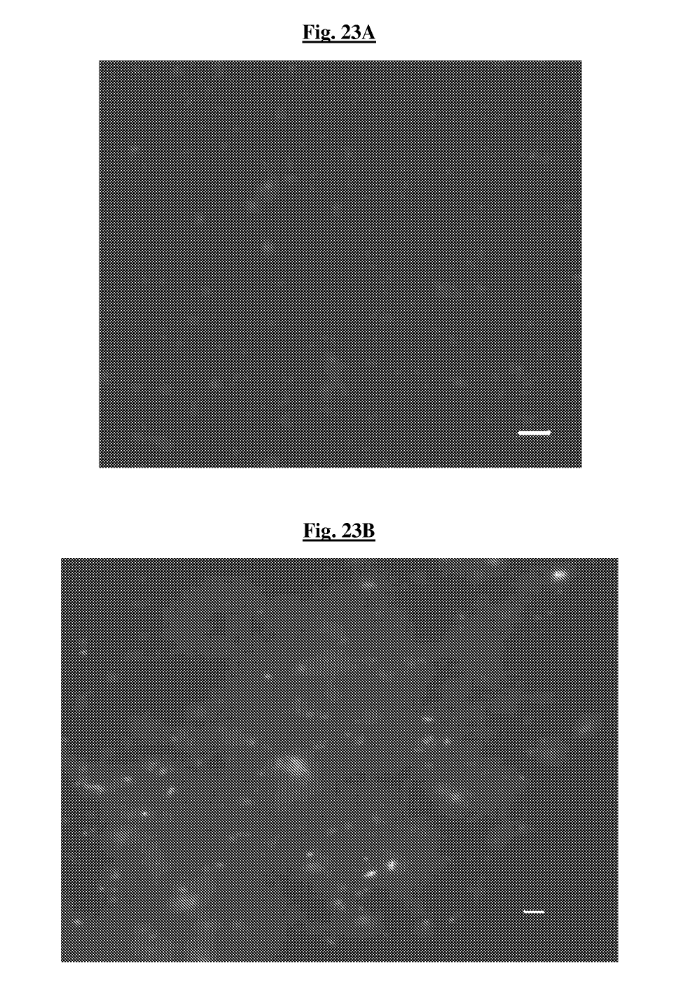

FIG. 21 depicts nanoparticles in the cell. The FITC panel in FIG. 22A suggests the presence of the unprocessed bevacizumab in the nucleus of the cell; however, the indirect labeling method makes it difficult to draw any conclusions since anti-human IgG-FITC has been shown to indiscriminately label the nucleus of cells. Further, the green stain could also be product of noise. To have a better understanding of the internalization behavior and to avoid controversy in terms of data interpretation, direct labeling of the particles with FITC prior to incubation with the cells was attempted.

### Direct Labeling of Nanoparticles

The nanoparticles and the unprocessed bevacizumab particles were successfully labeled with FITC as shown in the fluorescence micrographs in FIG. 23.

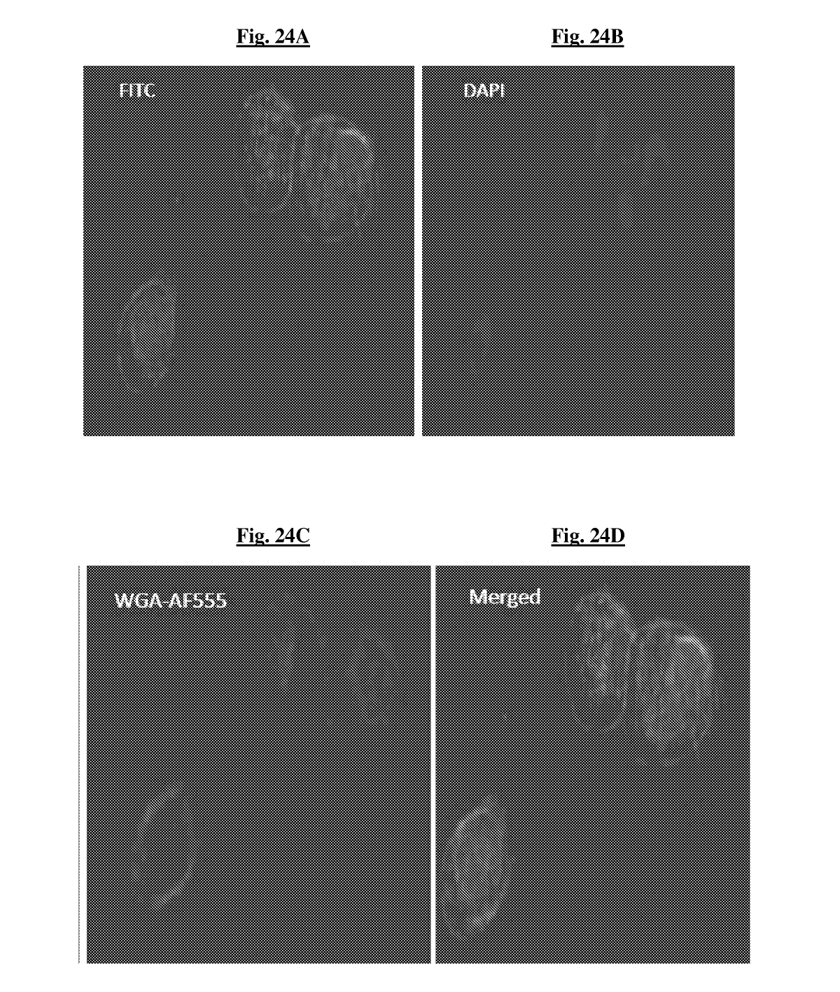

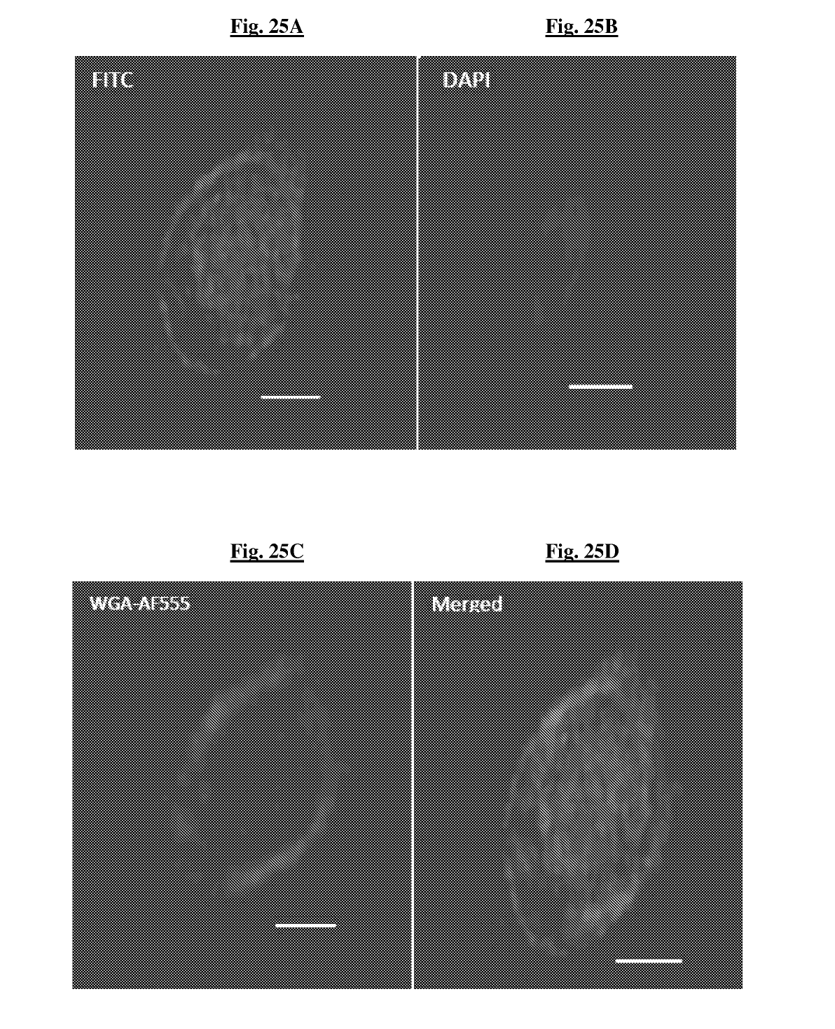

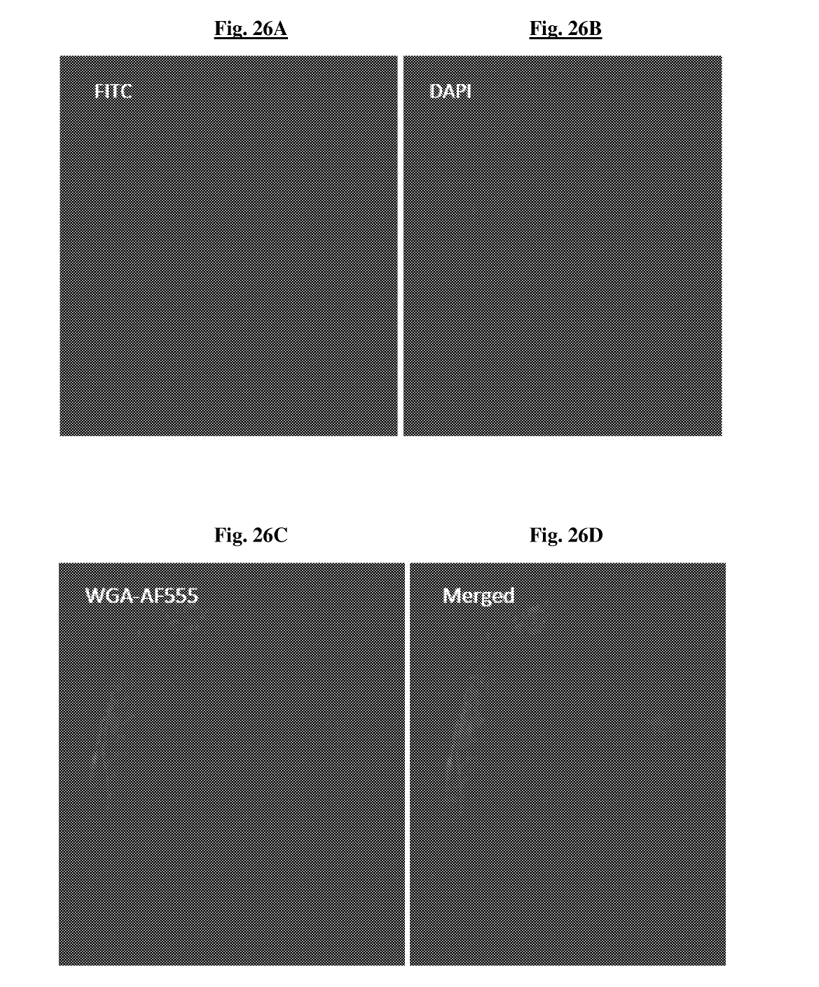

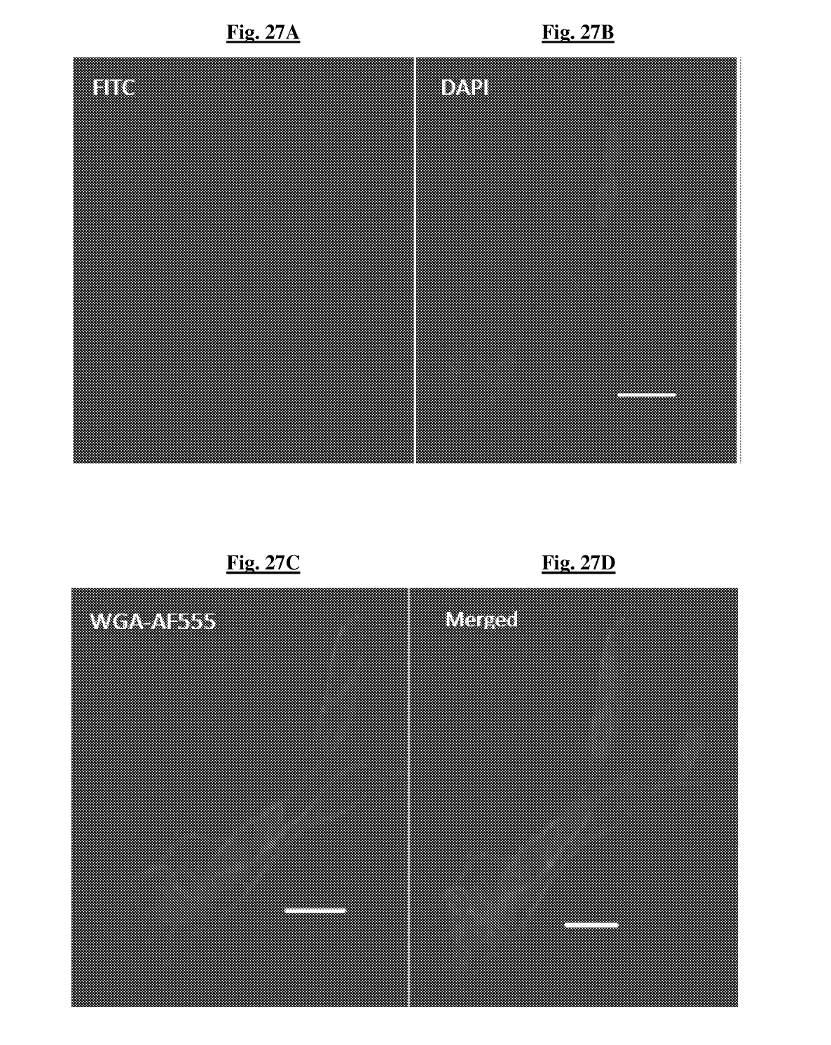

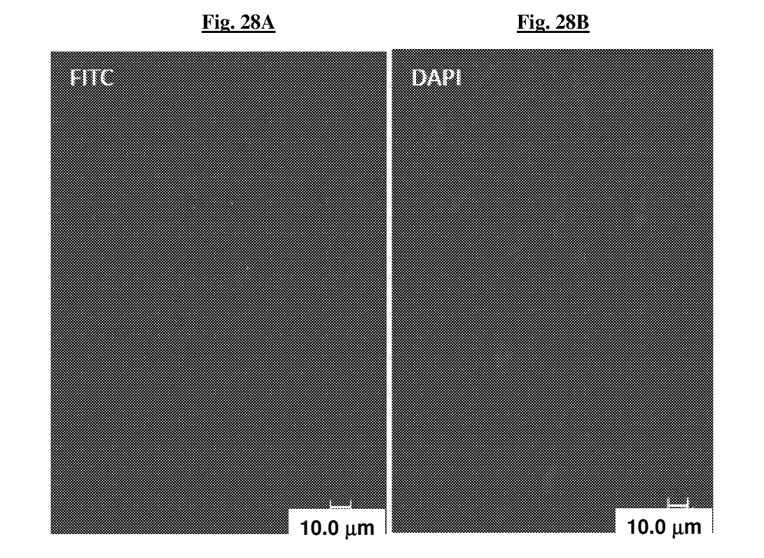

Following the incubation of the cells with FITC-labeled nanoparticles, the micrographs illustrated in FIGS. 24-27 were obtained from the fluorescence studies.

### Selectivity Between Cancer Cells (A549) and Normal Lung Fibroblast (MRC-5)

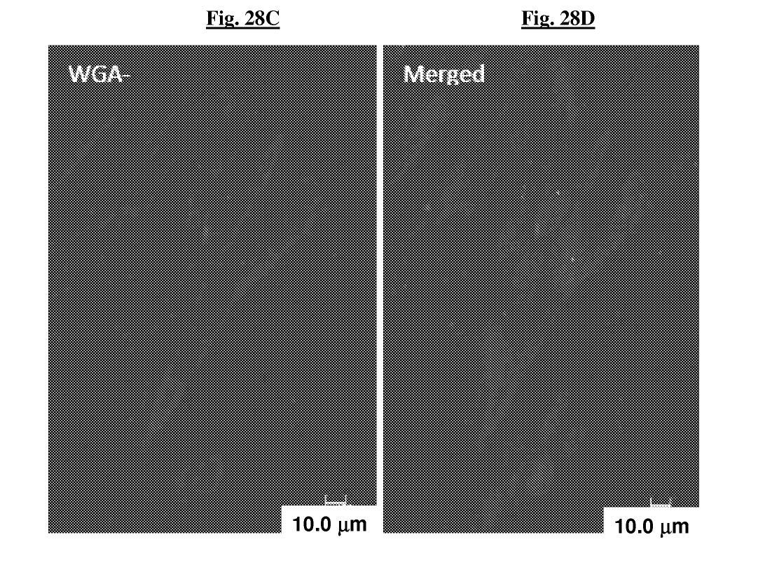

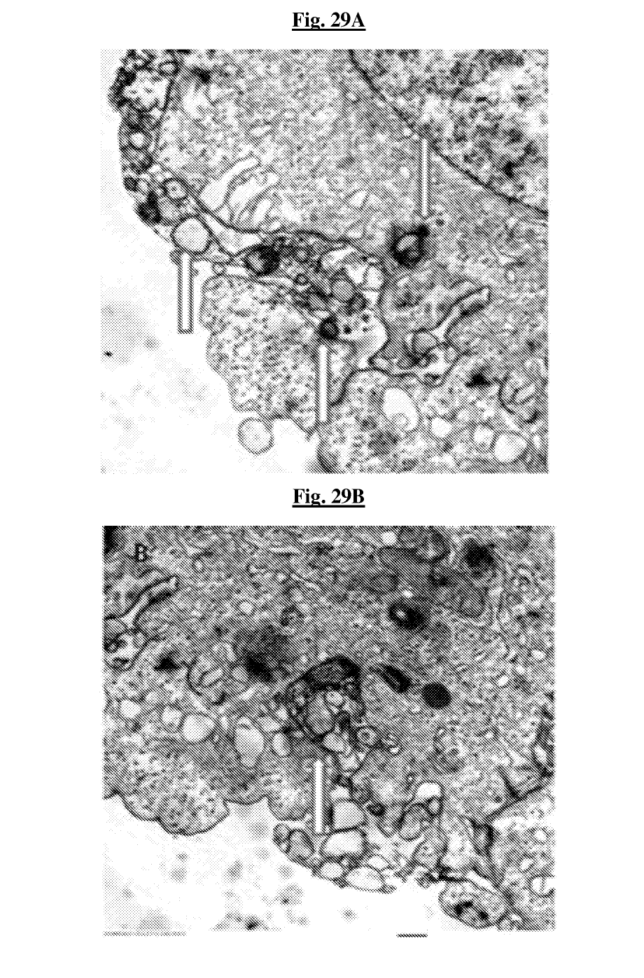

MRC-5 was incubated with the same concentration of nanoparticles used in the A549 experiment in order to investigate whether the nanoparticles selectively accumulate in cancer cells in comparison to normal lung fibroblast cells (FIG. 28).

### Transmission Electron Microscopy (TEM)

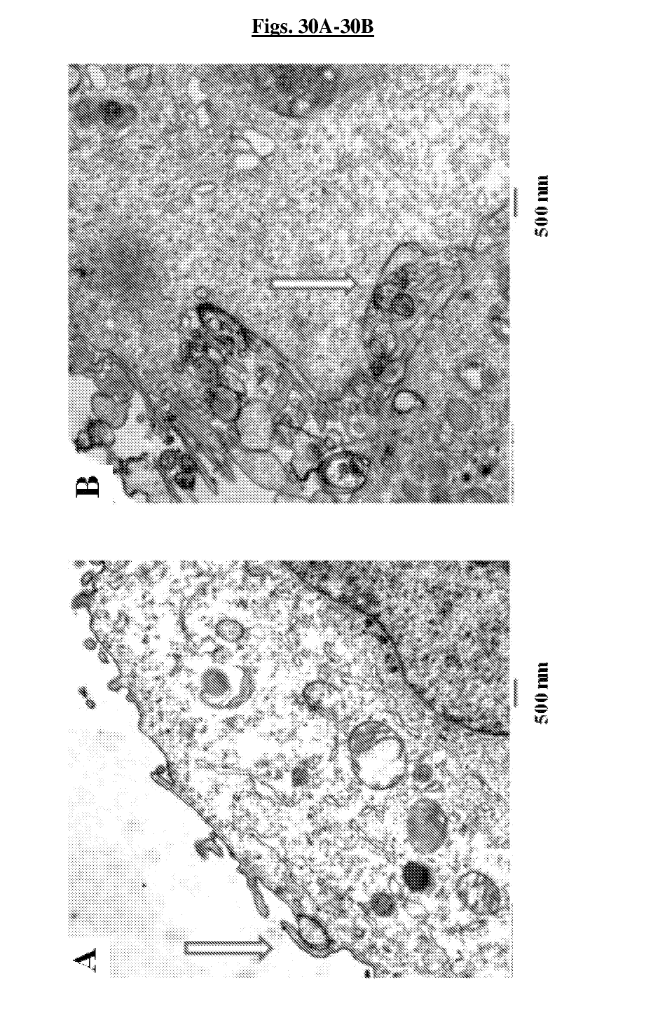

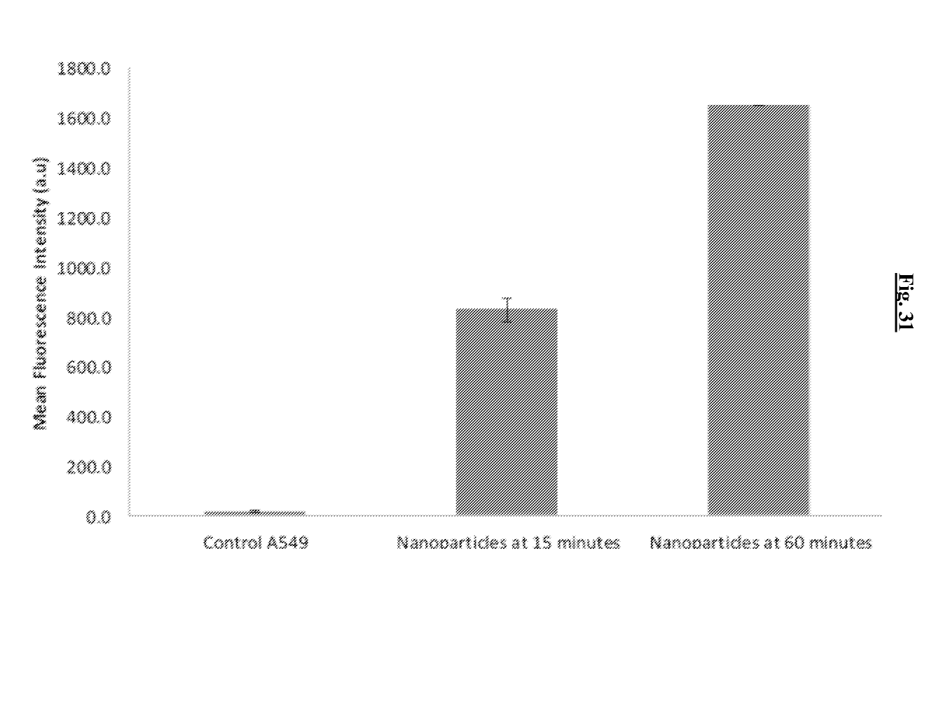

In order to have a preliminary understanding of the mechanisms of internalization of the nanoparticles by the A549 cells, the cells were incubated with the nanoparticles and were observed by TEM after 15 min, and 60 min (FIGS. 29 and 30).

The location of the nanoparticles in the endosome of cancer cells was further revealed in FIG. 30B. TEM data confirmed the data gained from flow cytometric analysis showing the involvement of macro-pinocytosis in the internalization process.

### Flow Cytometry

Statistical analysis using Student's t test shows no significant difference (P>0.05) in the uptake of FITC-labeled nanoparticles by the A549 cells initially treated with excess concentration of unlabeled bevacizumab and the cells not treated with excess concentration of unlabeled bevacizumab.

Flow cytometer was used to quantify the % particle internalized following incubation of cells with FITC labeled nanoparticles and to compare internalization between A549 and MRC-5.

A549 and MRC-5 cell lines were incubated with FITC-labeled nanoparticles for 15 and 60 minutes. The cells were then washed with D-PBS and extracellular florescence was quenched with trypan blue so that only internalized cells were analyzed. 10,000 cells were measured in each sample.

The amount of internalized nanoparticles was determined by measuring the mean fluorescence intensity (MFI) and converting it to % cellular internalization by normalizing with the sample with the highest MFI i.e. 0.1% Tween 80 bevacizumab nanoparticles after 60 minutes incubation with A549 (FIG. 31). Two controls were used in this experiment: (1) Cells (A549 and MRC-5) incubated with PBS and 01% Tween 80; and (2) Unstained cells (A549 and MRC-5) as instrumental controls.

FIG. 31 illustrates the lack of fluorescence in the control sample, indicating that the fluorescence observed in the nanoparticle incubated cells were indeed due to the nanoparticles. Further, increase in fluorescence was observed as the incubation increase from 15 minute to 60 minutes.

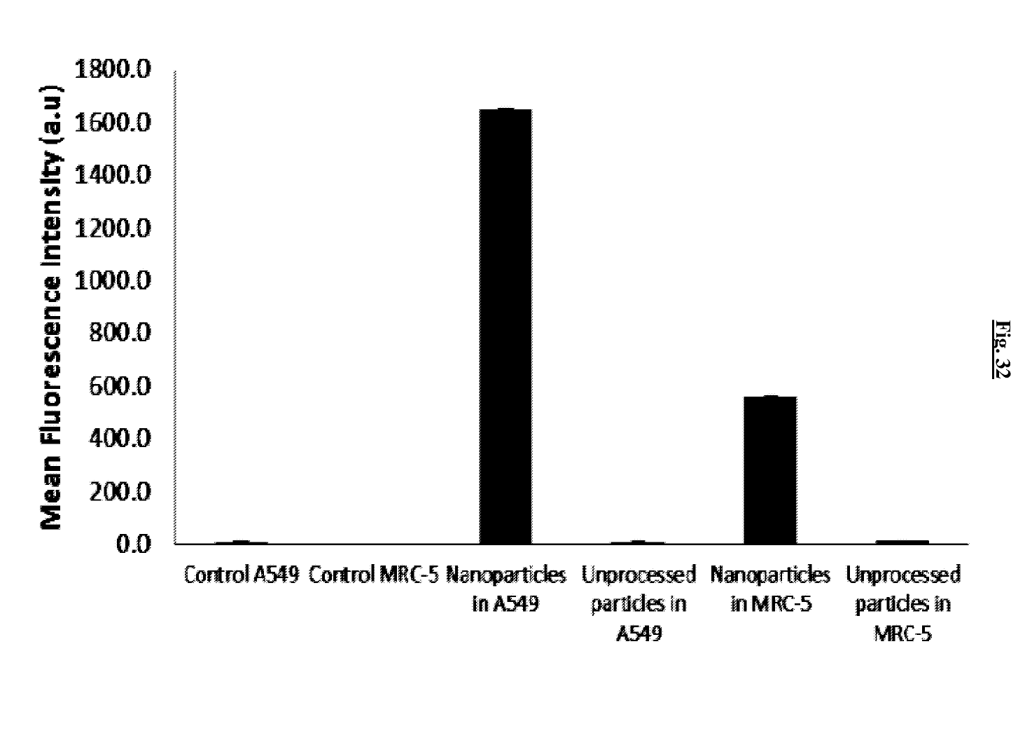

FIG. 32 illustrates selective accumulation of nanoparticles in A549 in comparison to MRC-5 cells.

### Internalization Pathways

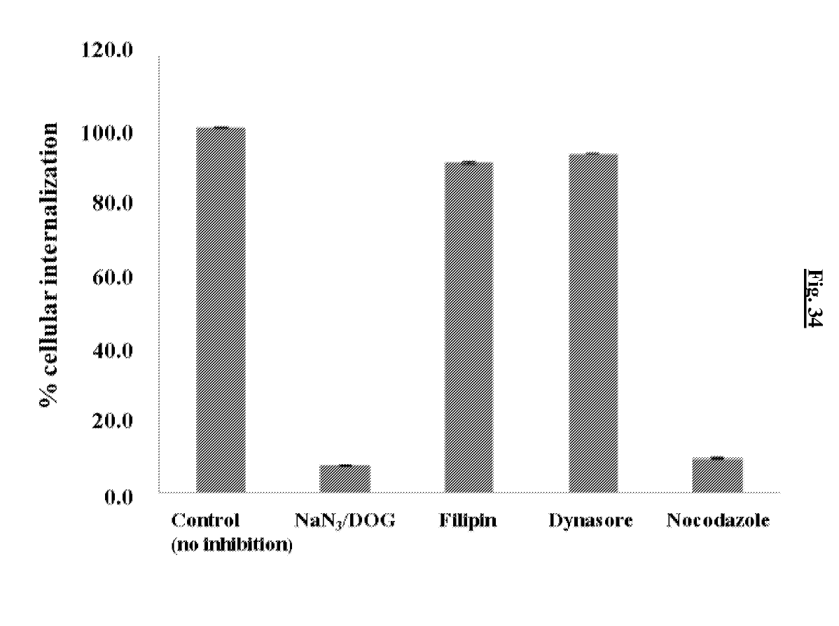

Elucidation of the pathways involved in the internalization of bevacizumab nanoparticles by A549 cells was carried out by using different chemical inhibitors (FIG. 34). NaN3/2-deoxyglucose (NaN₃/DOG) was used to assess energy-dependent pathway (Gratton et al., 2008, Pharrm. Res. 25:2845-2852). Filipin, a polyene antibiotic that sequesters plasma membrane cholesterol, was used to investigate particle internalization by a caveolae-mediated mechanism (Gratton et al., 2008, Pharrm. Res. 25:2845-2852). Dynasore, a cell permeable reversible inhibitor of dynamin, was used to probe internalization via clathrin-coated pits (Gratton et al., 2008, Pharrm. Res. 25:2845-2852). Nocodazole was used to inhibit macro-pinocytosis (Gratton et al., 2008, Pharrm. Res. 25:2845-2852). The mean fluorescence intensity (MFI) from the control (uninhibited A549 cells) was used to normalize the MFIs from the inhibited studies. FIG. 34 reveals that both energy-dependent endocytosis and macropinocytosis were the major pathways involved in the internalization of the nanoparticles by A549 cells.

Self-associated bevacizumab nanoparticles that could potentially be used for targeting an intracellular pool of VEGF in NSCLC were successfully prepared by a nanoprecipitation process. Bevacizumab was dissolved in 0.01 N HCl in order to facilitate the dissolution of bevacizumab. Due to the fact that the isoelectric point of bevacizumab is 8.4, an aqueous system with pH as far away from the isoelectric point as much as possible was used as to prevent uncontrolled precipitation of the protein. However, when subjecting mAbs or other proteins to such an extreme pH, a stabilizer/protectant may be added to the solution to prevent denaturation/degradation of the protein. In this case, non-ionic surfactants such as Tween 80, Tween 20, and Brij 97 were added to the system to help in the protection of the mAb against the low pH.

Nonionic surfactants may act as stabilizing agents for proteins by any of the following mechanisms. The stabilization may be attributed to the hydrophobic interaction of the non-ionic surfactants with protein molecules. The surfactants may form micelles around the bevacizumab molecules during the precipitation process, thereby protecting them from the harsh precipitating environment.

The increase in the size of bevacizumab nanoparticles as the surfactant concentration increased could be attributed to the hydrophobic interaction between the surfactants and the antibodies. Due to the fact that surfactants have a natural tendency to self-assemble into micelles at concentrations above the CMC, they may aid the self-association of the antibodies by including them in the self-assembly process since they exhibit hydrophobic interaction with the antibodies. The density of the negative charges on the particles also increased as the concentration of the surfactants increased. This is due to the preferential adsorption of the non-ionic surfactants on the positive charges in the antibody molecules leading to an increase in negative charges as the surfactant concentration increases. The zeta potential for the Tween 80 controls was approximately −13 mV, confirming that the surfactants were negatively charged under the precipitating conditions. The negative charge may be due to deprotonation (removal of H⁺) from the surfactant by the basic environment surrounding the surfactants.

The structural stability of bevacizumab following the nanoparticle formation process is relevant to the retention of their biological activity. A significant change in the 517 conformational structure of a protein often leads to a reduction or loss of biological activity. Perturbations in the secondary structure of bevacizumab following the reconstitution of the nanoparticles in 0.1 M acetate buffer at pH 5 were probed using far-UV circular dichroism (CD). pH 5 was chosen for reconstitution based on the hypothesis that the antibody nanoparticles would go into dissolution in the endosome of cancer cells at this pH (the pH of cellular endosomes has been reported to range between 4 and 6; Luzio et al., 2007, Nat. Rev. Mol. Cell Biol. 8:622-632; Blanchette et al., 2009, PLoS One 4:e6056, DOI: 10.1371/journal.pone.0006056; Cain et al., 1989, Proc. Natl. Acad. Sci. U.S.A. 86:544-548). The unprocessed bevacizumab had a predominantly β-sheet secondary structure. All the bevacizumab nanoparticles showed similar β-sheet secondary structure following reconstitution in 0.1 M acetate buffer (pH 5). Nevertheless, the surfactant-free bevacizumab nanoparticles showed major perturbations in the secondary structure as compared to the unprocessed bevacizumab, suggesting that the non-ionic surfactants are needed for maintaining the secondary structure of the antibody during the nanoparticle formation process and dissolution at pH 5. Nonionic surfactants are known to stabilize proteins against harsh conditions such as extreme pH under which the nanoparticles were produced. In certain embodiments, the perturbations in the secondary structure of the surfactant-free bevacizumab nanoparticles were due to the absence of the stabilizing effect of the non-ionic surfactants.

SE-HPLC was used to further investigate the physical stability of bevacizumab in the nanoparticles. The presence of aggregates and fragments in protein formulations is undesirable as it may signify the loss of stability. Further, the presence of aggregates in a reconstituted protein has been associated with immunogenicity. Prior to SE-HPLC analysis, bevacizumab nanoparticles were reconstituted in acetate buffer pH 5 so as to mimic the pH of the endosome where the nanoparticles are expected to go into dissolution. The levels of aggregates and fragments present in bevacizumab in all the surfactant-containing nanoparticles following reconstitution were minimal and comparable to that of the unprocessed bevacizumab. However, the surfactant-free bevacizumab nanoparticles contained a relatively higher level of fragments (14%), suggesting that the bevaicizumab in these nanoparticles was degraded/fragmented during the nanoprecipitation process due to the absence of surfactants. This result is consistent with the data obtained from CD spectroscopy, which showed some perturbation in the secondary structure of the surfactant-free bevacizumab. Fragment formation may be attributed to the hydrolysis of the peptide bonds by the HCl that was used to precipitate the particles.

Further, the stability of bevacizumab nanoparticles was confirmed by in vitro anti-VEGF activity of the reconstituted bevacizumab nanoparticles in HUVECs treated with hVEGF. The result suggested that surfactant is needed for maintaining the stability of bevacizumab during the nanoparticle formation process. The anti-VEGF activity of reconstituted bevacizumab nanoparticles was similar to that of unprocessed bevacizumab particles. The surfactant-free bevacizumab nanoparticles did not show a complete loss of activity as they seem to still retain some β-sheet in their secondary structure despite the perturbations. The secondary structure of the surfactant-free bevacizumab nanoparticles now contains a combination of random coil and β-sheet. However, these perturbations were not observed in the secondary structure of the surfactant-containing nanoparticles. Comparing the CD data to the activity data shows a correlation between the two.

Antiproliferative effect of reconstituted bevacizumab nanoparticle was confirmed in A549 cells. The in vitro effect of bevacizumab on the viability of the A549 NSCLC cell line was evaluated using a standard MTT assay. Bevacizumab nanoparticles produced cytotoxicity similar to that of the unprocessed bevacizumab in A549 cells. However, the surfactant-free bevacizumab nanoparticle formulation was not as effective. The presence of non-ionic surfactants in the surfactant-containing bevacizumab nanoparticles helps to maintain the integrity of the bevacizumab thereby retaining its cytotoxicity. However, surfactant-free bevacizumab lacks the stabilizing/protective ability of the non-ionic surfactants, and the loss of integrity as shown by CD and SE-HPLC may have impacted their cytotoxicity. Thus, a higher concentration of bevacizumab was needed for efficacy. This data is consistent with the results obtained from the in 600 vitro anti-VEGF assay.

Particle internalization was monitored using fluorescence 602 microscopy, TEM, and flow cytometry. Bevacizumab nanoparticles were substantially internalized in A594 cells. In these experiments both plasma membrane and the nucleus were stained to aid in differentiating membrane bound particles and internalized ones. The bevacizumab nanoparticles were not efficiently internalized by MRC-5 normal lung fibroblast cells. Fluorescence microscopy data correlated well with data generated from flow cytometry, which demonstrate that bevacizumab nanoparticles were internalized three times more in the A549 cells in comparison to the MRC-5 cells. The preferential accumulation of bevacizumab nanoparticles in the A549 cells could be attributed to the increased membrane permeability observed in cancer cells in comparison to normal cells. Compared to normal cells, the absence or dysfunction of tight junctions (TJ) in confluent cancer cells and the marked amounts of intracellular materials released from cancer cells into surrounding fluid provide corroborative evidence of increased permeability of the cancer cell membrane.

The nonspecificity of the uptake of self-associated bevacizumab nanoparticles was confirmed by initially treating the cells with excess concentration of unlabeled bevacizumab before treating with FITC-labeled nanoparticles. Treatment with excess bevacizumab would lead to the occupation of any targets on the cell membrane that could be responsible for the uptake of these nanoparticles. Subsequent treatment with FITC-labeled nanoparticles should have led to a significant reduction in the uptake of the FITC-labeled nanoparticles. However, this was not the case. The implication of this data is that other mAbs may be internalized as self-associated nanoparticles by cancer cells.

A series of chemical inhibitors were used to elucidate the endocytic pathways used by the A549 cells to internalize these self-associated bevacizumab nanoparticles. The results obtained suggest that a mixture of both energy-dependent endocytosis and macropinocytosis was involved in the internalization process. Energy-dependent process as inhibited by NaN₃/DOG and macropinocytosis as inhibited by nocodazole were found to decrease drastically by approximately 95%. However, clathrin-coated pit pathway (inhibited by dynasore) and caveolae-mediated pathways were only inhibited by approximately 10%. The fact that both caveolae and clathrin pathways were not involved in the internalization process is in total agreement with current understanding that these two pathways have an upper limit of 200 nm. It should be noted that additional mechanisms of internalization exist such as non-clathrin, non-caveolae mediated endocytosis, which were not screened in the inhibition studies. Such mechanisms could account for supplementary pathways of internalization.

Further insight into the cellular internalization behavior of these associated bevacizumab nanoparticles was gained by TEM. The extension of the plasma membrane around the nanoparticles in a one-handed manner as the particles are internalized indicates macropinocytosis. The noninvolvement of phagocytosis in the internalization process may be attributed to the fact that A549 cells are nonphagocytic.

In one aspect, self-associated bevacizumab nanoparticles were successfully prepared by a nanoprecipitation process. The stability of bevacizumab in these nanoparticles was confirmed using far-UV 663 CD and SE-HPLC. Retained in vitro anti-VEGF activity of bevacizumab in these nanoparticles was investigated and found to be comparable to that of the unprocessed bevacizumab particles. The bevacizumab nanoparticles were found to be taken up by A549 cells three times more than MRC-5 cells. This study presents evidence that uncoated mAb nanoparticles can be selectively delivered to cancer cells while avoiding normal cells.

### Example 4

### Survivin

Survivin is a unique member of the inhibitor of apoptosis (IAP) protein family that interferes with post-mitochondrial events including activation of caspases in cancer. The isoelectric point (pI) of anti-survivin mAb was measured using isoelectric focusing, and was determined to be 7.4. Using the methods described elsewhere herein, self-associated nanoparticles of anti-survivin mAb with particle size approximately 200 nm were prepared.

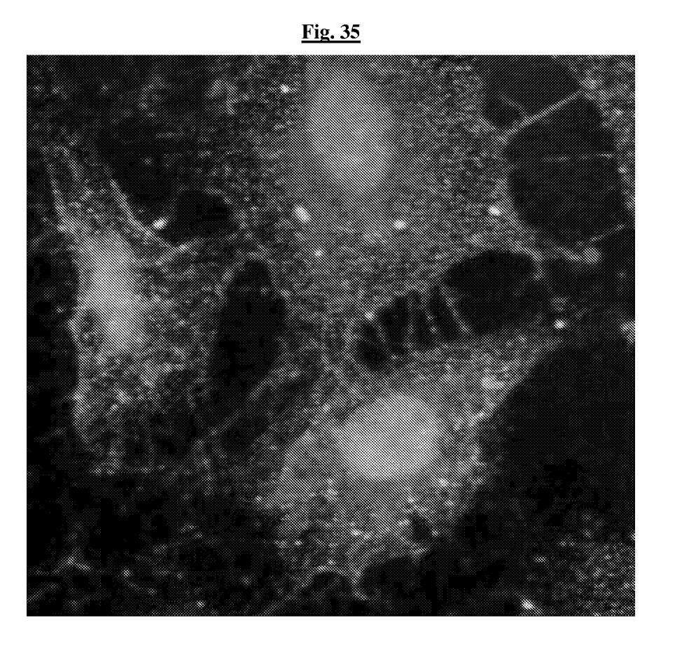

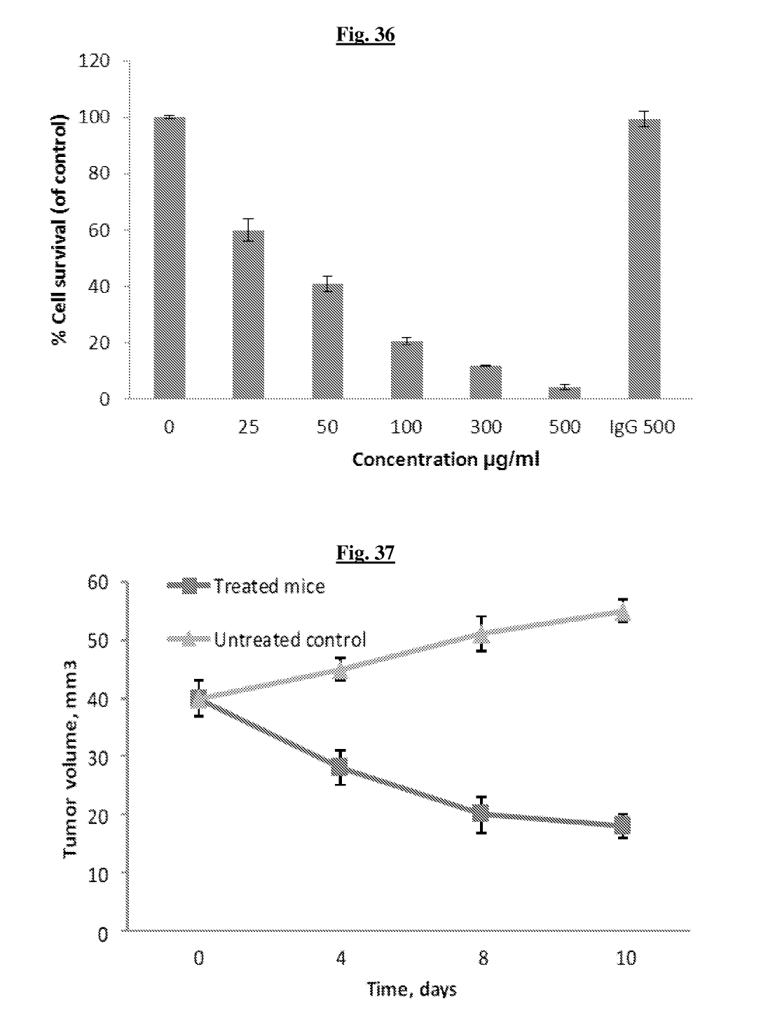

The uptake of anti-survivin nanoparticles by A549 cells was evaluated using confocal micrography (FIG. 35). The results indicate that self-associated anti-survivin mAb nanoparticles labeled with FITC were internalized by the cells. FIG. 36 illustrates the dose-dependent in vitro anti-proliferative effect of the anti-survivin nanoparticles against A549 cells. FIG. 37 illustrates the time-dependent in vivo anti-tumor effect of injected anti-survivin nanoparticles in the mouse.

### Example 5

### Carrier System

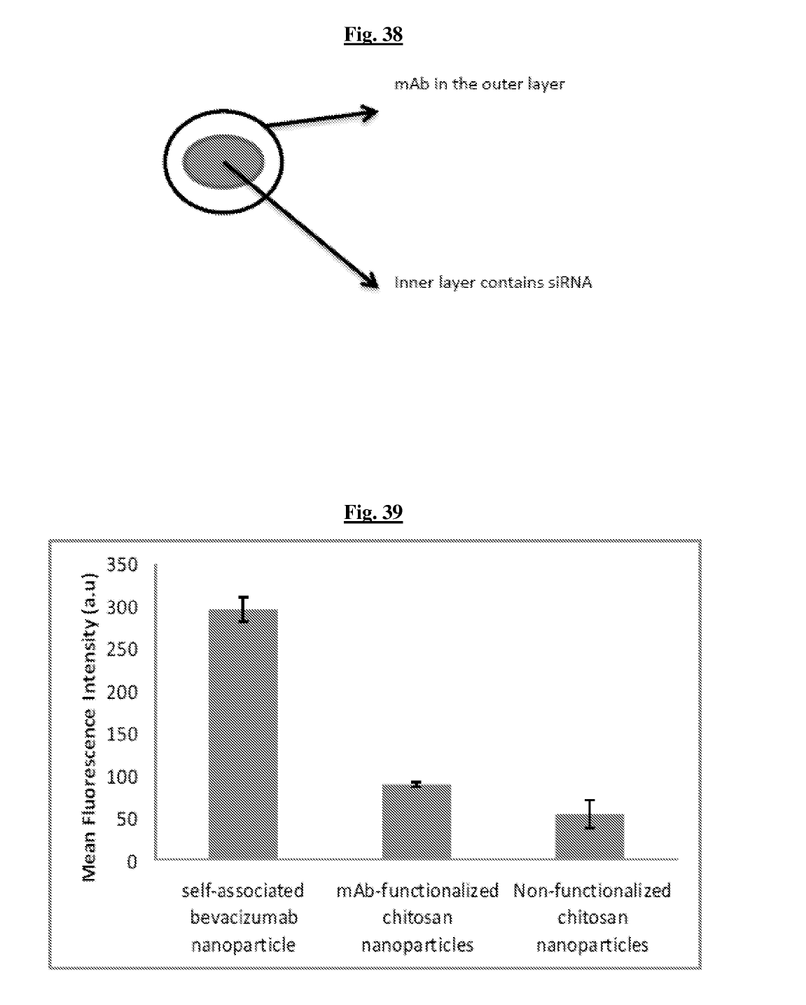

The compositions of the present invention may be employed a carrier system for delivering a composition to a cell. In certain embodiments, the composition comprises a nucleic acid or a small molecule. A non-limiting schematic of a siRNA-loaded mAb nanoparticles is illustrated in FIG. 38.

The uptake of self-associated MAb nanoparticles by A549 cells was compared to the uptake of chitosan nanoparticles by the same cells (FIG. 39). A549 cells were incubated with the same concentrations (100 μg/ml) of nanoparticles before flow cytometry analysis. As illustrated in FIG. 39, the uptake of self-associated mAb nanoparticles was higher than that of mAb-functionalized chitosan nanoparticles or non-functionalized chitosan nanoparticles.

### Example 6

### Non-Limiting Illustration of the Preparation and Characterization of Self-Associated Nanoparticles of Monoclonal Antibodies and/or Proteins

With the objective of preparing and characterizing self-associated nanoparticles of monoclonal antibodies and/or proteins, the precise isoelectric point of the monoclonal antibody and/or protein is determined using isoelectric focusing. All excipients are removed from the antibody by using 30 kDa cutoff centrifugal ultrafilters (Millipore Corporation) at 4000 g and 10° C. for 15 minutes. Double distilled deionized water is added to the antibody left in the inner tube of the dialysis, and the antibody and/or protein is lyophilized to get dry powder.

Alternatively, the antibody and/or protein is provided in powder form and free of any excipient.

Distinct concentrations of polysorbate 80 (Tween 80), polysorbate 20 (Tween 20), and/or Brij 97 are dissolved in 0.01N hydrochloric acid. In certain embodiments, the concentration of surfactant used is 0.1-0.3% w/v. Antibody (5 mg/ml or 7.5 mg/ml or 10 mg/ml) is added to the mixture. In certain embodiments, 5 mg/ml antibody is added to the mixture.

The mixture is slowly titrated to the pH of the isoelectric point of the antibody and/or protein with 0.01N sodium hydroxide solution. In certain embodiments, the mixture turns slightly cloudy, confirming the formation of the nanoparticles. The colloidal suspension is then centrifuged using a microcentrifuge at 6,500 rpm for 5 min.

The supernatant is decanted, and the pellet formed is rinsed thrice with double distilled deionized water. The nanoparticles are resuspended by adding double distilled deionized water and vortexing is used to resuspend the nanoparticles. The nanoparticles are dried by freeze drying or spray drying. The dried nanoparticles may be formulated into any dosage form needed.

A high concentration colloid formulation may be prepared by suspending the required concentration of antibody in phosphate buffered saline. The dry powder formulation may be stored as such.

The stability of the antibody and/or protein in the nanoparticles may be determined using CD, SEC-HPLC, FTIR or mass spectrometry. The particle size of the nanoparticles formed can be measured using zetasizer.

### Example 7

### Stable and Efficient Transfection of siRNA for Mutated KRAS Silencing

### Methods

Preparation of siRNA-Loaded Nanoparticles

15 mg of excipient-free human IgG were dissolved in 0.01 N HCl containing 6 mg of poloxamer-188 and 45 μg of siRNA to make a 3 ml total solution. This was then slowly titrated with 0.01N NaOH to bring the pH of the mixture to 7, which is the isoelectric point (pI) of human IgG, as determined using isoelectric focusing. The mixture was continuously mixed on a magnetic stirrer. At the pI, siRNA-loaded nanoparticles spontaneously precipitated.

The colloidal suspension was then centrifuged with a microcentrifuge (Eppendorf Centrifuge 5418) at 2000 rpm for 5 min. The supernatant was decanted, and the nanoparticles rinsed thrice with double distilled deionized water. Nanoparticles were then redispersed in water before being snap-frozen using liquid nitrogen. This was then loaded into a freeze-dryer (Labconco FreezeZone 4.6) and lyophilizer was performed for 48 h.

### Nanoparticle Characterization

Particle size and zeta potential of the nanoparticles were measured by photon correlation spectroscopy (PCS) using ZetaSizer Nano ZS (Malvern Instruments, UK). Pellets formed after centrifugation and rinsing were redispersed in deionized water and sonicated for approximately 5 min. Intensity autocorrelation was measured at a scattering angle (θ) of 173°. The Z-average and polydispersity index (PDI) were recorded in triplicate. For zeta potential, samples were taken in a universal dip cell (Malvern Instruments) and the zeta potential recorded in triplicate.

The morphology and internal arrangements of the components of the nanoparticles were characterized by transmission electron microscopy (TEM). A drop of nanoparticle suspension was made on to a copper grid coated with carbon membrane and then air dried. The nanoparticles were observed using a FEI Tecnai 12 TEM. Electron micrographs were captured with an AMT XR111 11 megapixel CCD camera.

In Vitro siRNA Release from Nanoparticles

Release of siRNA from both IP-nanoparticles and IgG-nanoparticles was investigated using pH 5 and 7 acetate buffer and PBS respectively. The freeze dried nanoparticles were suspended in 0.5 mL of a buffered solution in a tubular cellulose dialysis membrane secured tightly at both ends, and the system was then incubated in 5 mL buffered solution reservoirs at 37° C. while gently agitating the reservoir. The amount of siRNA released at different time points was analyzed and quantified for percentage cummulative release using ion-pair HPLC.

Ion pair HPLC

siRNA analysis was performed using an Alliance HPLC system; Waters 2695 separation module combined with a Water 2998 photodiode array detector (Waters, Milford, Mass., USA). A Waters XSELECT™ HSS C18 column XP (4.6×150 mm) was used. 1 μl of siRNA sample was injected using 20 Mm triethylamine-acetic acid (pH 7) and 5-12% acetonitrile, gradient elusion as mobile phase. Analysis was performed at a flow rate of 0.2 mL/min. UV detection was performed at 269 nm and chromatogram were recorded using Empower Pro software.

siRNA Stability in Serum

IgG-poloxamer-188 nanoparticles (IP-nanoparticles) loaded with siG12S, IgG nanoparticles loaded with siG12S and naked siG12S were incubated in F12K medium containing 50% FBS at 37° C. Aliquots of 5 μl samples were taken at time points up to 48 h. Samples were immediately mixed with 2% SDS, and 5 μl TBE urea sample buffer. The mixture was then heated at 70° C. for 3 minutes in order to deactivate the nuclease enzyme in the serum. The integrity of the siRNA was then analyzed using 15% TBE-ureal gel. siRNA bands were visualized by ethidium bromide staining.

### Cellular Uptake and Intracellular Trafficking of Nanoparticles

Cellular uptake and trafficking of siRNA-loaded nanoparticles was monitored by loading the nanoparticles with siGLO-Green (6-FAM-labelled), and incubating with A549 lung cells. A549 cells (2×10⁴/well) were seeded in 8 well coated slides (Discovery Labware, USA) and incubated for 24 h. The medium was then aspirated. PBS washed cells were incubated with siGLO-loaded nanoparticles for a total of 6 h. Cells were washed with PBS and fixed with 4% paraformaldehyde at 1, 2, 4 and 6 h. PBS washed cells were then blocked with 5% BSA for 30 min at room temperature. PBS washed cells were stained with LysoTracker® Red and DAPI. Cells were mounted and observed under a Leica DMI 6000B fluorescence microscope (Leica Microsystems, Exton, Pa.).

### In Vitro Gene Silencing Studies

A549 cells were transfected 24 h after seeding in a 6-well plate at density of 2×10⁵/well. Naked siG12S and control siRNA (Thermal Scientific, Amarillo, Tex.) were transfected with lipofectamine RNAimax transfection reagent (Invitrogen). siRNA-loaded IP and IgG nanoparticles were incubated with the cells at 37° C. in a humidified air environment with 5% carbon dioxide for 96 h. The total concentration of siRNA used in all experiments was 50 nM.

### Western Blot Analysis

Cells were washed twice with ice-cold PBS and then lysed in M-Per mammalian lysis buffer (Thermo Scientific). The protein concentration of the lysates was determined with the Bradford reagent (Bio Rad), and equal amounts of protein were subjected to SDS-PAGE using a 10% gel. Separated proteins were transferred to a nitrocellulose membrane, which was then exposed to 5% nonfat dried milk in TBS containing 0.1% Tween 20 (0.1% TBST) for 1 h at room temperature and incubated overnight at 4° C. with antibodies against KRAS (Abcam, Cambridge, Mass.) or actin (Sigma). The membranes were then washed with 0.1% TBST before being incubated with horseradish peroxidase-conjugated goat antibodies to rabbit (Santa Cruz Biotechnology). Immune complexes were detected with chemiluminescence reagents (Perkin-Elmer Life Science).

Quantitative Real Time Polymerase Chain Reaction (qRT-PCR)

A549 cells were seeded at a density of 2.5×10⁵ in 6 well plate, incubated at 37° C. in a humidified air environment for 24 h. PBS-washed cells were then treated with the respective nanoparticles and incubated for another 72 h. Total RNA was harvested using RNAase isolation kit (Qiagen Inc.). Complementary DNA (cDNA) was synthesized from 200 ng of total RNA using verso cDNA kit (Thermo Fischer Scientific, MA, USA). Complementary DNA was used to carry out the real time PCR of kRAS by TaqMan gene expression assay system (Applied Biosystems, Life Technologies) according to the manufacturer's instructions. Wild-type KRAS siRNA was used as scramble (control) for this experiment Glyceraldehyde 3-phosphate dehydrogenase (GAPDH) expression was used to normalize mRNA expression.

### Cytotoxicity Assay

MTT assay was used to determine the effect of siG12S loaded nanoparticles on the proliferation of A549 cells. Chemosensitivity of siG12S-treated cells to erlotinib was also assessed. Cells (1×10⁴ per well) were seeded in 96 well plates and incubated at 37° C. in a humidified atmosphere with 5% carbon dioxide for 24 h. The cells were then treated with different formulations of nanoparticles, siG12S and erlotinib. Cells were incubated for 72 h. 10 μL of 12 Mm MTT reagent were then added to each well. This was then incubated at 37° C. for 4 h. The medium was aspirated and 50 μL of sterile DMSO was added to each well and mixed thoroughly with pipet. The cells were then incubated at 37° C. for 10 min. The plate was read at 540 and 650 nm.

### Immunogenicity and Inflammatory Reaction in Murine Macrophages

RAW 264.7 murine macrophage cells were seeded in a 96 well plate at a density of 1.5×10⁵/well at 37° C. in humidified air with 5% carbon dioxide for 24 h. The cells were then treated with 100 μg/mL of LPS, naked siG12S, siG12S-loaded IP-nanoparticles, siG12S-loaded IgG-nanoparticles and culture medium seperately for 24 h. TNF-α and IL-6 concentrations in supernatant from cultured cells were analyzed using the respective ELISA kit according to the manufacturer's instructions.

### Phagocytosis in Murine Macrophages

Phagocytosis in RAW 264.7 murine macrophage was investigated by seeding 2×10⁴ cells/well in 8 well coated slides (Discovery Labware, USA) and incubated for 24 h. The cells were then treated with Fluospheres® Red beads (positive control) and FITC-labeled nanoparticles for a total of 4 h. Cells were washed with PBS and fixed with 4% paraformaldehyde at 2 and 4 h. PBS washed cells were blocked with 5% BSA for 30 min at room temperature. PBS washed cells were stained DAPI. Cells were mounted and observed under a Leica DMI 6000B fluorescence microscope (Leica Microsystems, Exton, Pa.).

### Statistical Analysis

Results are expressed as mean±standard deviation, unless otherwise indicated. Statistically significant difference between two groups was determined by two-tailed Student's t test. A p-value of 0.005 was taken as statistically significantly.

### Experimental Examples

### Characterization of Nanoparticle

Nanoparticles were prepared using the methods described herein. As seen in Table 11, IgG-nanoparticles prepared without siRNA had particle size of 249.4±23.7 nm and a zeta potential of 19.7±1.0 mV. Following the loading of siRNA, the particle size increased to 426.4±19.9 nm and a shift in surface charge to 17.4±0.4 mV. IgG-poloxamer-188 (IP) nanoparticles produced a particle size of 412.5±34.2 nm with a zeta potential of 19.4±2.7 mV. The loading of siRNA led to an increase in particle size (672.4±17.9 nm) and a shift in surface charge to 17.1±0.8.

TABLE 11
Particle size, PDI, and Zeta potential analysis of nanoparticles
measured by photon correlation spectroscopy (PCS)

| Nanoparticle | Particles size (nm)^{a} | PDI^{a} | Zeta Potential (mV)^{a} |
|---|---|---|---|
| IgG | 249.4 ± 23.7 | 0.08 ± 0.03 | 19.7 ± 1.0 |
| siRNA-IgG | 426.4 ± 19.9 | 0.30 ± 0.03 | 17.4 ± 0.3 |
| IP | 412.5 ± 34.2 | 0.06 ± 0.01 | 19.4 ± 2.7 |
| siRNA-IP | 672.4 ± 17.9 | 0.08 ± 0.02 | 17.1 ± 0.8 |

^{a}Data presented as mean ± SD

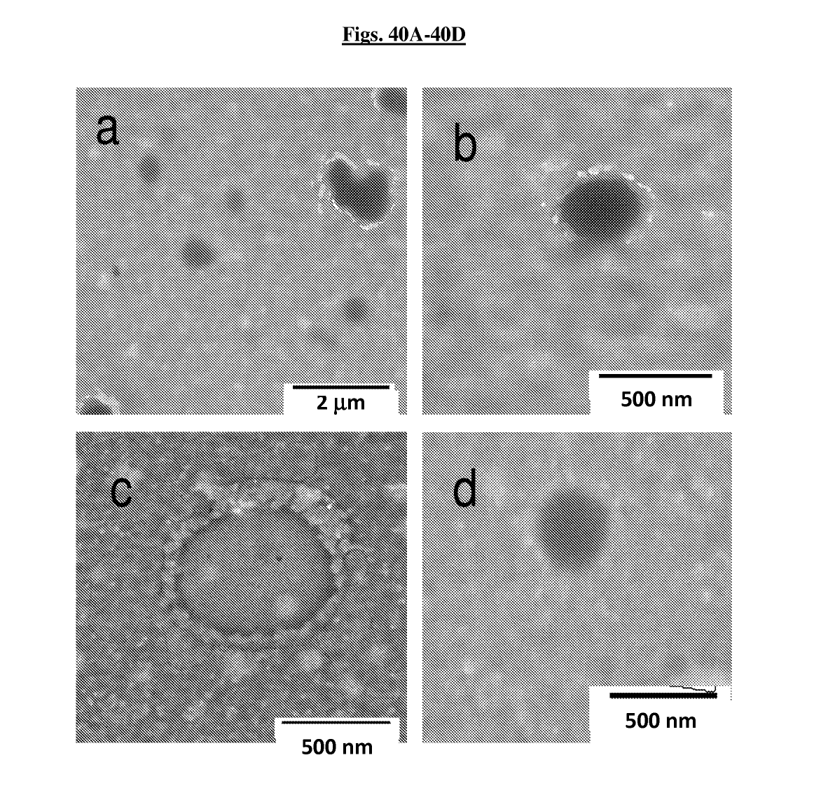

TEM micrographs in FIG. 40 illustrate that the nanoparticles were generally spherical in shape. FIG. 40A illustrates the nanoparticles at lower magnification, while FIG. 40B illustrates siRNA-free IgG-nanoparticle. FIG. 40C illustrates siRNA-PI nanoparticles with the siRNA in the inner-core, while the next layer contain IgG surrounded by poloxamer-188 on the outside layer.

siRNA Encapsulation Efficiency

Encapsulation efficiency (EE) and loading capacity (LC) of siRNA in the nanoparticles were measured using ion-pair-high performance liquid chromatography (IP-HPLC). Linearity was established for siRNA for concentrations ranging from 0 to 50 μM. Following the preparation of nanoparticles, filtrates were taken from the centrifuged samples and analyzed using IP-HPLC. EE and LC were calculated from the following equations:

% EE=(A−B)/A×100 and % LC=(A−B)/C×100

where A=total amount of siRNA, B=free siRNA, and C=weight of nanoparticles in grams.

The encapsulation efficiency of siRNA was approximately 41% while loading capacity was approximately 0.70% irrespective of the type of nanoparticles.

### Assay of Serum Stability

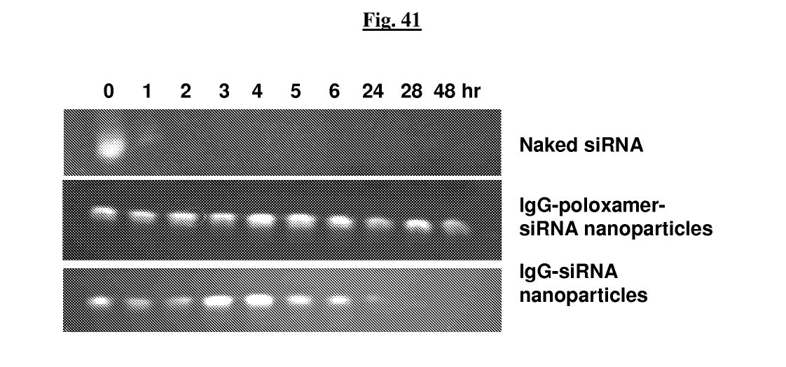

Serum stability was assessed by incubating the naked siRNA and siRNA-loaded nanoparticles in 50% serum and the integrity of the siRNA assessed using gel electrophoresis at different time points. FIG. 41 illustrates the finding that naked siRNA was intact immediately after incubation but degraded after 1 h. In contrast, siRNA in IP nanoparticles was intact after 48 h. siRNA in IgG-nanoparticles was also intact up to 24 h. However, IgG-nanoparticles were not able to protect the siRNA after 24 h.

In vitro siRNA Release from Nanoparticles

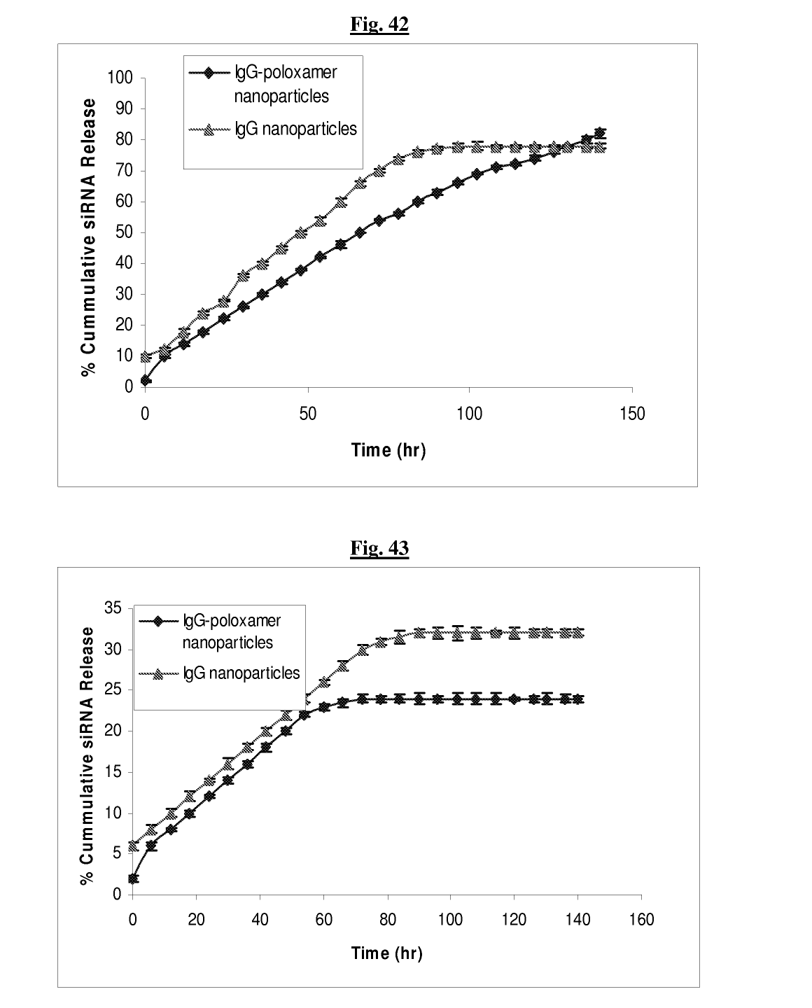

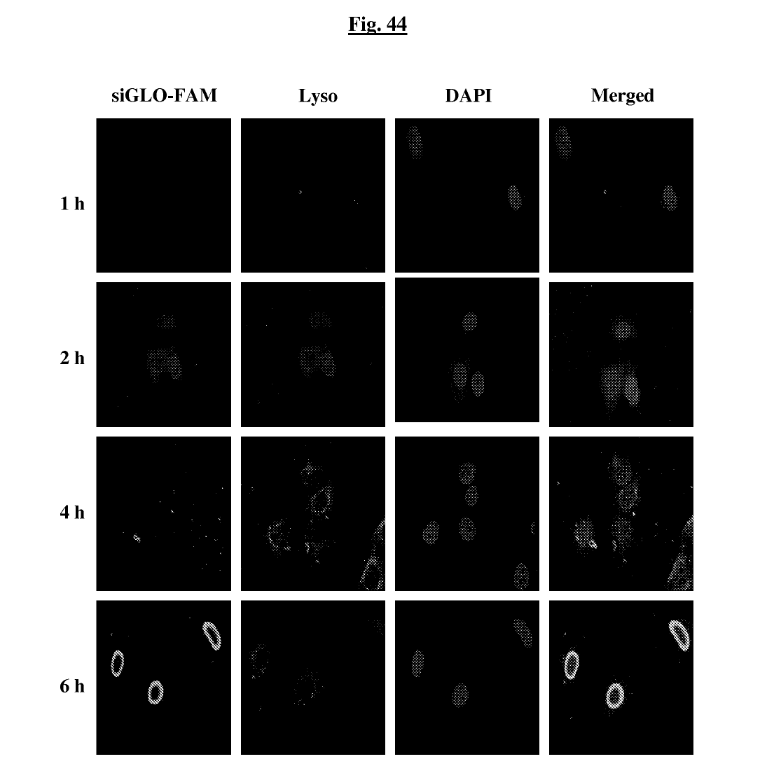

At pH 5, the addition of poloxamer-188 in the IP-nanoparticles drastically reduced the initial burst experienced in the IgG-nanoparticles (FIG. 42). While 10% of siRNA release was observed at 0 h for IgG-nanoparticles, only 2% release was observed for IP-nanoparticles. Over a period of 30 h, more than 36% siRNA was released from IgG-nanoparticles, while only 26% was released from IP-nanoparticles. Similar trend was observed at pH 7 (FIG. 43) although the overall release of siRNA was significantly reduced when compare to pH 5.

### Intracellular Trafficking of Nanoparticles

Intracellular location of siGLO-IP nanoparticles was examined by tracking the siGLO-FAM with fluorescent microscopy (FIG. 44). Lysotracker Red was used to stain late endosomes/lysosome. At 1 h post incubation, there were no siGLO observed in the cells. However, at 2 hr post incubation, maximum level of co-localization of siGLO and lysotracker was observed, suggesting the presence of the siGLO-IP nanoparticles in the late endosome. After 4 h, the siGLO-FAM was observed to be leaving the late endosome and moving into the cytoplasm, as indicated by the green color surrounded by lysotracker-red. Full localization of siGLO inside and around the nucleus was observed after 6 h, completing the transfection of siGLO-FAM.

In vitro Gene Silencing

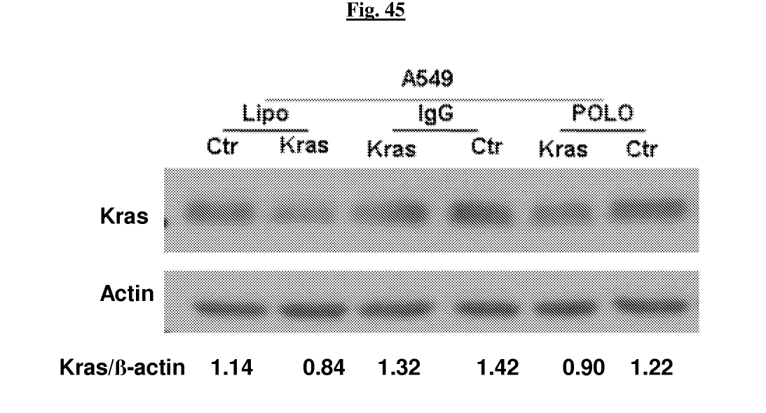

KRAS levels in A549 cells were measured by western blot analysis after treatment with different nanoparticle formulations. As illustrated in FIG. 45, a major reduction in KRAS expression was observed in the siG12S-treated cells when compared to the control-treated cells. Lipofectamine, a commonly used transfection agent was used as a comparative control. Importantly, no significant difference was observed in the efficiency of lipofectamine and that of IP-nanoparticles in the knockdown of KRAS expression.

qRT-PCR

FIG. 45 demonstrates that siG12S-IP nanoparticles significantly (P<0.005) reduced the expression of KRAS-mRNA compared to the control which was the untreated cells. The scramble-IP nanoparticles did not significantly (P>0.005) reduce the expression of KRAS-mRNA in comparison to the control.

### Cytotoxicity

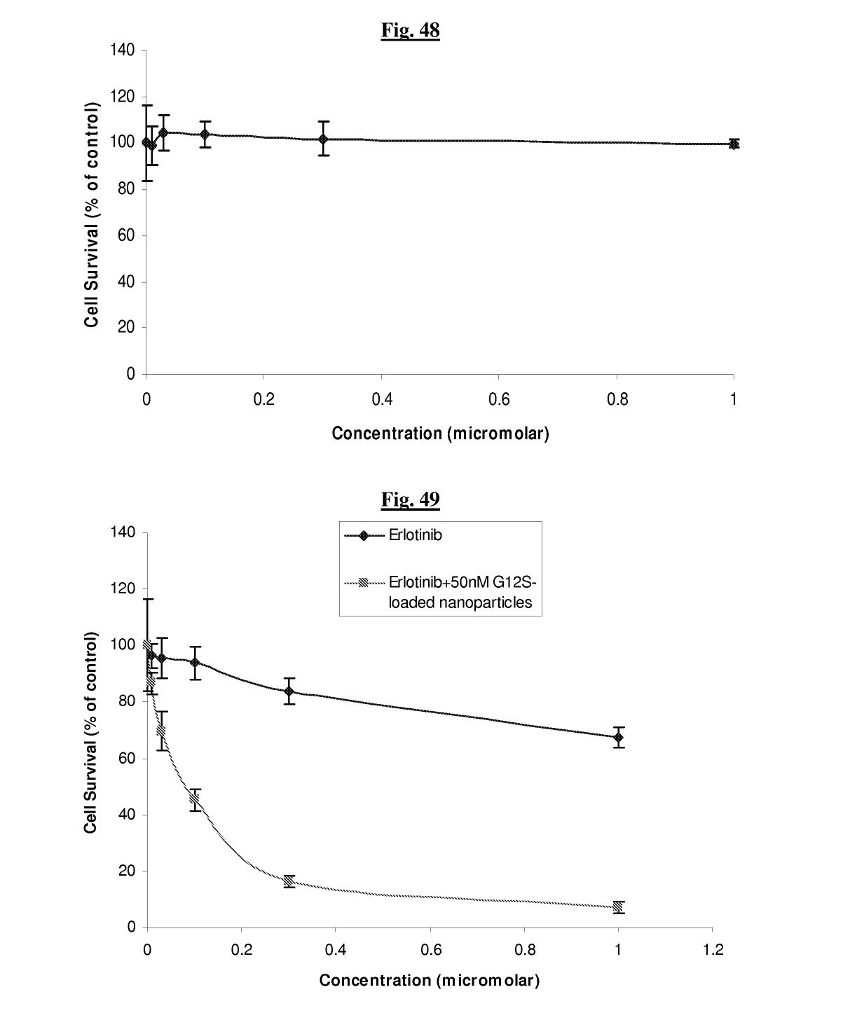

MTT assay was used to investigate the cytotoxicity of various siRNA/nanoparticle formulations. All formulations contained 50 nM of siRNA. Chemisensitivity of siG12S-treated A549 cells to erlotinib was also investigated. FIG. 47 illustrates the finding that IP-scramble nanoparticles did not inhibit the growth of A549 cells. Likewise, naked siG12S did not inhibit the growth of the cells. However, the growth of IP-siG12S treated cells was significantly (P<0.005) inhibited as compared to the control. FIG. 48 illustrates the non-toxicity of plain IP-nanoparticles. Cytotoxicity of different concentrations of erlotinib was compared to that of a combination of erlotinib and siG12S-IP nanoparticles to determine the sensitivity of A549 cells to erlotininb following KRAS knockdown by siG12S. FIG. 49 illustrates the finding that A549 cells were resistant to erlotinib if applied alone. However, following the knockdown of KRAS in A549 cells by siG12S-IP nanoparticles, a significant sensitivity to erlotinib was demonstrated by A549 cells.

### Immunogenicity and Inflammatory Reaction in Murine Macrophages

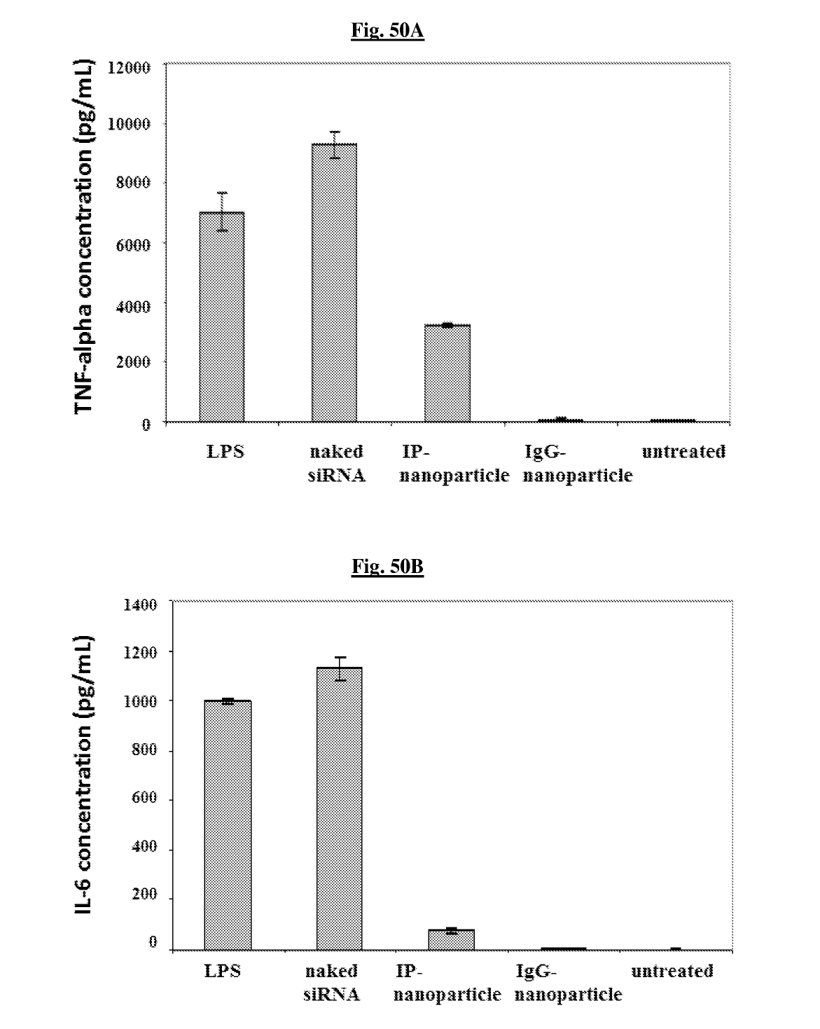

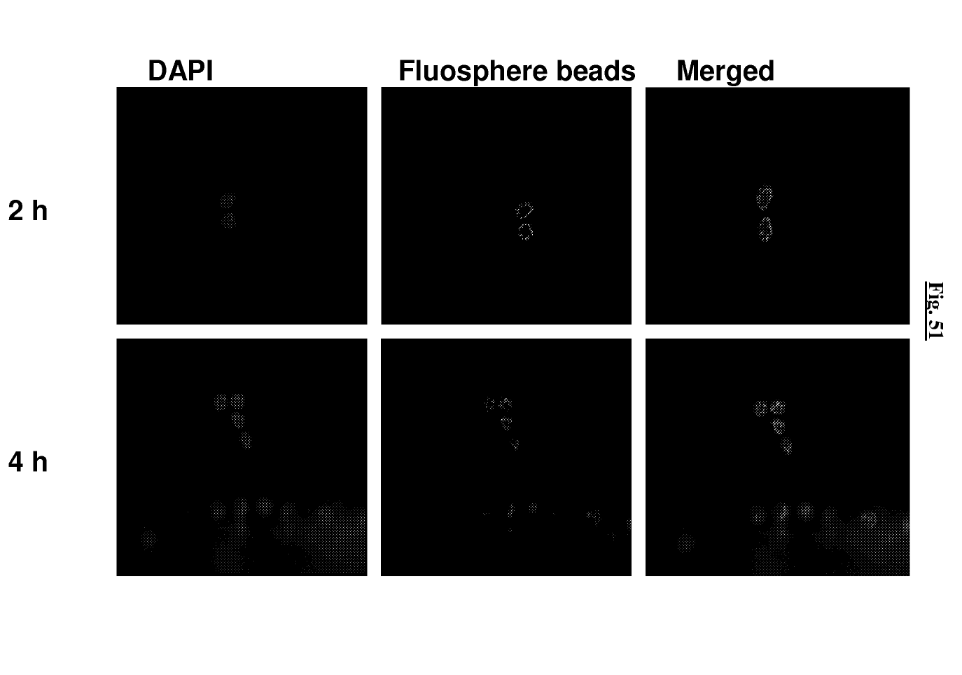

The immuno-stimulatory effects of naked siG12S, siG12S-IP and siG12S-IgG nanoparticles were evaluated and compared to that of LPS, which is a known immuno-stimulatory agent. FIG. 50 illustrates the finding that naked siG12S upgraded the production of both TNF-α and IL-6 even more so than LPS. However, siG12S-IP and siG12S-IgG nanoparticles significantly (P<0.005) downgraded the production of the two agents.

### Phagocytosis in Murine Macrophages

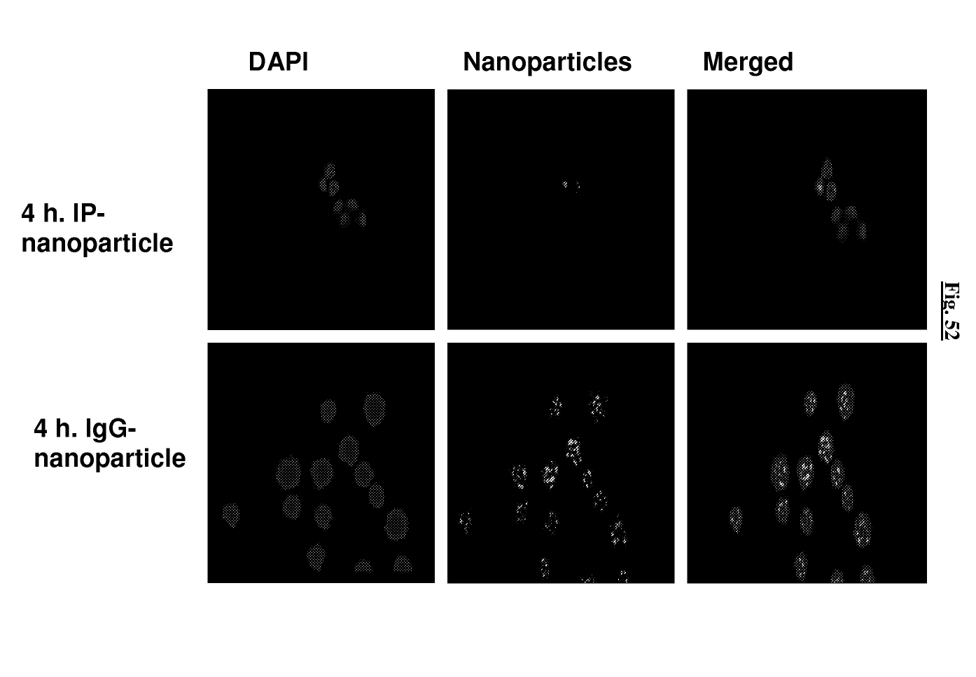

FIG. 51 illustrates the finding that the latex beads (fluospheres) were phagocytosed in large numbers by the macrophages after 2 h, and even more so after 4 h. However, the IP-nanoparticles and IgG-nanoparticles were not phagocytosed at all after 2 h, as no green dye was observed in the fluorescence image. However, after 4 h, very minor amount of IP-nanoparticles was engulfed by the macrophages (FIG. 52). In contrast, IgG-nanoparticles were engulfed in large numbers after 4 h by the macrophages (FIG. 52)

As demonstrated herein, a novel hybrid nanoparticle delivery system for stable and efficient delivery of siG12S for effective knockdown of mutant KRAS in lung carcinoma cells was developed. KRAS encodes a GTP-binding protein that is involved in cellular processes such as proliferation, differentiation and apoptosis. However, KRAS mutations are detected in more than 25% of lung adenocarcinomas. These mutations are associated with poor prognosis in non-small lung cancer patients (NSCLC). Further, KRAS mutation is associated with poor response to EGFR-TKIs therapy. Thus, mutant KRAS inhibition is critical to successful NSCLC treatment. Several strategies such as farnesyltransferase inhibitors have been explored as possible inhibitors of KRAS in lung adenocarcinomas. To date, none of these strategies have proved to be successful mainly due to non-specificity. Often times the undesirable inhibition of the wild type KRAS is also achieved.

RNAi using siRNA is effective and specific in the knockdown of specific mutant KRAS. Nevertheless, issues still remain on the ideal delivery system for safe and effective delivery of these siRNAs to cancer cells. Lipid nanoparticles undergo endocytic recycling leading to the loss of approximately 70% of the internalized siRNA. Lack of effective transfection, stimulation of immune/inflammatory reaction, clearance by RES and degradation by endonuclease in the blood are some of the other limitations of other delivery systems that have been recently investigated.

To achieve a successful delivery of siG12S, a novel hybrid nanoparticles comprising human IgG and poloxamer-188 was developed. siG12S is an siRNA designed specifically for the knockdown of mutant KRAS_{G12S} that is normally expressed in A549 cells. Nanoparticles were prepared based on the fact that proteins have minimum solubility but maximum precipitation at the isoelectric point. In certain embodiments, a concentration of 5 mg/ml antibody was used as the starting material.

TEM data (FIG. 40) confirmed that a multilayer nanoparticle system with poloxamer-188 forming the outermost layer of the nanoparticle was achieved. The multilayered nature of the nanoparticles provided extra protection against serum nuclease, making it possible for the encapsulated siG12S to survive nuclease degradation for at least 48 h. Encapsulated siG12S was also protected by IgG-nanoparticles, but not as effectively as observed in IP-nanoparticles. The lack of protection for the naked siG12S (FIG. 41) was mainly responsible for the quick degradation observed in this formulation. In certain embodiments, a multilayered nanoparticle system allows for an effective protection of siRNAs against blood nuclease.

In vitro release studies were carried at pH values 5 and 7.2, in order to simulate some of the conditions to be encountered by this formulation in vitro. pH 5 represents the acidic condition of the endosome/lysosome. Extracellular fluids are known to have a neutral pH (approximately 7). The release of siG12S from IgG-nanoparticles at pH 5 (FIG. 42) showed an initial burst, which was prevented by the addition of poloxamer-188 to the IP-nanoparticles. Without wishing to be limited by any theory, the initial burst in the IgG-nanoparticles could be attributed to the adsorption of some of the siG12S on the surface of the nanoparticles, which was released quite easily by diffusion. However, the presence of poloxamer-188 in the IP-nanoparticles probably helped to prevent the adsorption of siG12S on the surface of the nanoparticles.

FIG. 43 demonstrates the pH sensitivity of the nanoparticle formulations. At pH 7, only 22% of siG12S was released from IP-nanoparticles, while approximately 34% was release from IgG-nanoparticles. Without wishing to be limited by any theory, yhe limited release of siG12S at pH 7 could be attributed to the reduced/limited solubility of IgG at pH. Proteins are known to have limited solubility at pH values close to their isoelectric point (pI), and the solubility of IgG at neutral pH values is quite limited (pI of IgG is 7). This makes it almost impossible for encapsulated siRNAs to be released extracellularly.

Intracellular trafficking of IP-nanoparticles was monitored using fluorescence microscopy. Following internalization of siRNA-loaded nanoparticles, the loaded siRNA should find its way into the cytoplasm of the host cell. However, the majority of internalized materials are targeted to the lysosomes for degradation, and endocytic recycling accounts for the loss of approximately 70% of internalized lipid nanoparticles due to exocytosis from the endosome. FIG. 44 illustrates the finding that siGLO was successfully released into the cytoplasm of A549 cells at approximately 4 h, while the siGLO found its way to the nuclease at approximately 6 h. This was possible due to the buffering capacity of the IP-nanoparticles in the endosome. Without wishing to be limited by any theory, the solubility of IgG and poloxamer-188 at acidic pH, as demonstrated by the in vitro release data in FIG. 42, made it possible for the dissolved IgG in the endosome to activate the proton pump that raises osmotic pressure in the endosome, subsequently leading to the swelling and subsequent escape of siGLO from the endosomes into the cytoplasm.

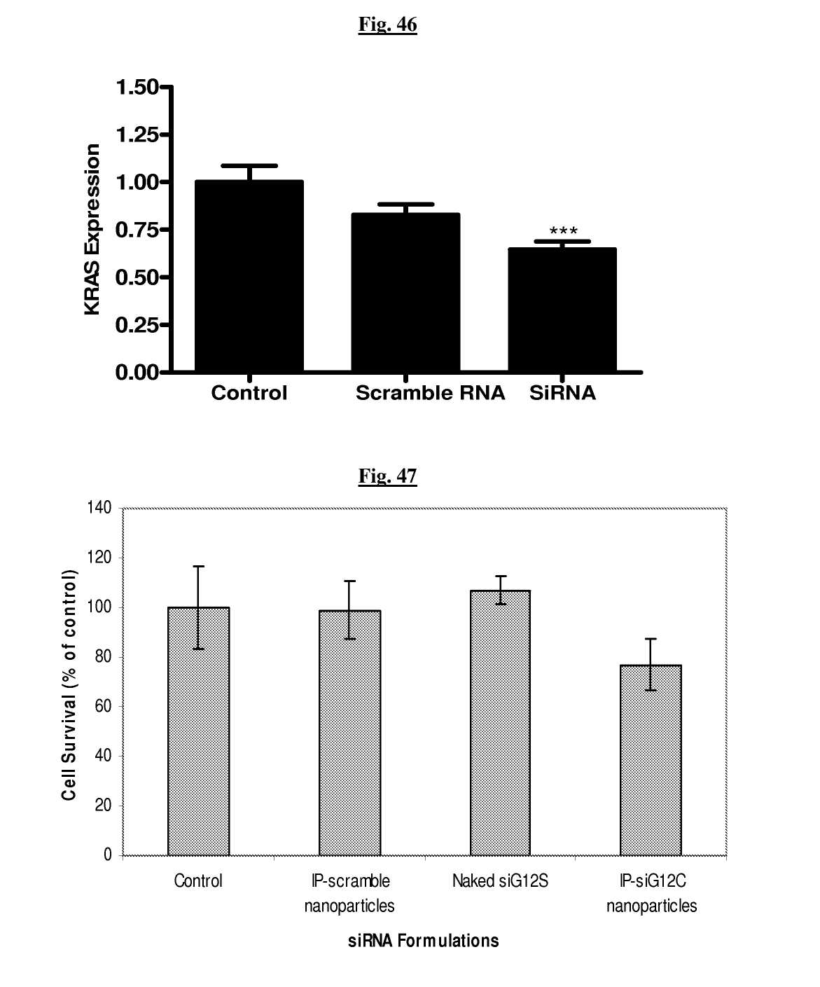

In vitro gene silencing capability of encapsulated siG12S in IP-nanoparticles was demonstrated to be comparable to the universally used lipofectamine. IgG-nanoparticles were not as effective in silencing KRAS^{G12S} probably because of the relative susceptibility of encapsulated siG12S to nuclease in the FBS used in the culture medium. A total knockdown of KRAS could not be achieved probably due to the concentration of siG12S (50 nM) used in these experiments; an increase in concentration could have led to a total knockdown. RT-PCR was also used to determine the level of mRNA-KRAS in the treated A549 cells compared to the untreated cells. A significant knockdown of mRNA-KRAS by siG12S-IP-nanoparticles was observed. siRNA against the wild-type KRAS (WT-KRAS) was used as scrambled-control in this experiment. WT-KRAS siRNA was used so as to determine whether this siRNA would also lead to an efficient knockdown of mRNA-KRAS in mutant-KRAS expressing A549 cells. As shown in FIG. 46, a non-significant knockdown of mRNA-KRAS was observed, suggesting that some WT-KRAS was still being expressed by A549 cells. It should be noted that some NSCLC cell lines carry both mutant and WT-KRAS, and siRNA against mutant-KRAS did not lead to complete loss of cell viability in resistant NSCLC due to retained expression of WT-KRAS, which permits the survival of NSCLC cells through normal regulation of signal transduction.

KRAS mutation in NSCLC causeses increased resistance to EGFR-TKIs. Hence, a knockdown of mutant-KRAS in A549 cells may lead to increased sensitivity to EGFR-TKIs. FIG. 49 illustrates the finding that A459 cells had increased sensitivity to erlotinib following concurrent treatment with siG12S-IP-nanoparticles. FIG. 47 illustrates the finding that siRNA against mutant-KRAS did not lead to complete loss of cell viability in resistant NSCLC. Further, as illustrated in FIG. 48, the IP-nanoparticles alone were non-toxic to A549 cells.

The present study demonstrated that the nanoparticles of the present invention not only efficiently transfect siRNAs but to do so in a safe non-toxic way. Nanoparticles previously used to deliver siRNAs to cells were limited by their ability to stimulate immune response. The present nanoparticles comprise IgG, a part of the body's natural defense mechanism, and poloxamer-188, a stealth polymer that prevents macrophageal uptake of nanoparticles. As demonstrated herein, IgG-nanoparticles loaded with siG12S showed no immunostimulatory effect, as the level of TNF-α and IL-6 released by the IgG-nanoparticle treated macrophages cells was as low as the negative control (untreated cells). LPS is a known immunostimulatory agent hence was used as a positive control in this experiment. I P-nanoparticles loaded with siG12S had a significantly reduced immunostimulatory effect as compared to the naked siG12S LPS, probably in part because of the presence of IgG in the nanoparticle formulation. However, relatively low immunostimulatory effect observed in the IP-nanoparticles treated macrophage cells could also be attributed to the presence of poloxamer-188 in the nanoparticle formulation. Clearance by macrophages of the RES is a well-known limitation of nanoparticles in the delivery of siRNA for therapeutic purposes. In this study, inclusion of poloxamer-188 helped prevent uptake of the nanoparticles by macrophages.

The disclosures of each and every patent, patent application, and publication cited herein are hereby incorporated herein by reference in their entirety. One skilled in the art will readily appreciate that the present invention is well adapted to carry out the objects and obtain the ends and advantages mentioned, as well as those inherent therein. While the invention has been disclosed with reference to specific embodiments, it is apparent that other embodiments and variations of this invention may be devised by others skilled in the art without departing from the true spirit and scope used in the practice of the present invention. The appended claims are intended to be construed to include all such embodiments and equivalent variations.

What is claimed is:

1. A composition comprising at least one protein nanoparticle, wherein the protein nanoparticle is prepared by a method comprising adjusting the pH of a solution comprising a protein and a non-ionic surfactant to about the isoelectric point of the protein, wherein the concentration of the non-ionic surfactant in the solution ranges from about 5% to about 20,000% of the CMC of the surfactant, thereby forming a precipitate comprising the protein nanoparticle, wherein the protein nanoparticle comprises at least a fraction of the protein and at least a fraction of the non-ionic surfactant.

2. The composition of claim 1, wherein the protein comprises at least one selected from the group consisting of an antibody, hormone, immunomodulator, cytokine, interferon, interleukin, and enzyme.

3. (canceled)

4. The composition of claim 2, wherein the antibody comprises IgG or a monoclonal antibody.

5-6. (canceled)

7. The composition of claim 4, wherein the monoclonal antibody comprises at least one selected from the group consisting of bevacizumab, anatumomab, benralizumab, enokizumab, mitumomab, oxelumab, and palivizumab.

8. The composition of claim 1, wherein the solution further comprises at least one additional compound selected from the group consisting of a therapeutic agent and a cell surface receptor ligand, and the protein nanoparticle comprises at least a fraction of the at least one additional compound.

9. The composition of claim 8, wherein the at least therapeutic agent is selected from the group consisting of an organic compound, inorganic compound, pharmacological drug, radiopharmaceutical, protein, peptide, polysaccharide, nucleic acid, siRNA, RNAi, short hairpin RNA, antisense nucleic acid, ribozyme and dominant negative mutant.

10-11. (canceled)

12. The composition of claim 8, wherein the at least one ligand binds to at least one selected from the group consisting of neurotensin receptor-1, human epidermal growth factor receptor-2 (HER-2), folate receptor, insulin-like growth receptor (IGF), and epidermal growth factor receptor (EGFR).

13. The composition of claim 1, wherein the non-ionic surfactant comprises at least one selected from the group consisting of an alkyl polyethylene oxide, alkylphenol polyethylene oxide, copolymer of polyethylene oxide and polypropylene oxide, alkyl polyglucoside, fatty alcohol, cocamide MEA, cocamide DEA, and polysorbate.

14. The composition of claim 13, wherein the alkyl polyethylene oxide comprises at least one selected from the group consisting of a diethylene glycol hexadecyl ether, polyethylene glycol oleyl ether, diethylene glycol octadecyl ether, polyoxyethylene stearyl ether, polyethylene glycol hexadecyl (cetyl) ether, polyethylene glycol dodecyl (lauryl) ether, decaethylene glycol oleyl ether, polyethylene glycol octadecyl ether, and polyethylene glycol octadecyl ether.

15. The composition of claim 1, wherein the average diameter of the at least one protein nanoparticle is selected from the group consisting of: from about 1 nm to about 1,000 nm, and from about 100 nm to about 900 nm.

16. (canceled)

17. The composition of claim 1, wherein the concentration of the non-ionic surfactant in the solution is selected from the group consisting of: from about 100% to about 20,000% of the CMC of the surfactant, from about 300% to about 10,000% of the CMC of the surfactant, and from about 300% to about 5,000% of the CMC of the surfactant.

18-20. (canceled)

21. A method of preparing at least one protein nanoparticle, the method comprising the steps of:
adjusting the pH of a solution comprising a protein and a non-ionic surfactant to about the isoelectric point of the protein, wherein the concentration of the non-ionic surfactant in the solution ranges from about 5% to about 20,000% of the CMC of the surfactant, thereby forming a precipitate comprising the at least one protein nanoparticle;
wherein the at least one protein nanoparticle comprises at least a fraction of the protein and at least a fraction of the non-ionic surfactant.

22. The method of claim 21, wherein the precipitate is further purified to remove protein or non-ionic surfactant that is not associated with the at least one protein nanoparticle, thereby generating a composition comprising the at least one protein nanoparticle.

23. (canceled)

24. The method of claim 21, wherein the protein comprises at least one selected from the group consisting of an antibody, a hormone, immunomodulator, cytokine, interferon, interleukin, and enzyme.

25-35. (canceled)

36. The method of claim 21, wherein the average diameter of the at least one nanoparticle ranges from about 1 nm to about 1,000 nm.

37. The method of claim 21, wherein the concentration of the non-ionic surfactant in the solution ranges from about 100% to about 20,000% of the CMC of the surfactant.

38. (canceled)

39. A method of treating, ameliorating or preventing a disease or disorder in a subject in need thereof, comprising administering to the subject a pharmaceutically effective amount of a composition comprising at least one protein nanoparticle comprising a protein and a non-ionic surfactant,
wherein the at least one protein nanoparticle is precipitated from a solution comprising the protein and the non-ionic surfactant, further wherein the pH of the solution is equal to about the isoelectric point of the protein, and further wherein the concentration of the non-ionic surfactant in the solution ranges from about 5% to about 20,000% of the CMC of the surfactant; and
further wherein the composition is administered to the subject by an intrapulmonary, intrabronchial, inhalational, intranasal, intratracheal, intravenous, intramuscular, subcutaneous, topical, transdermal, oral, buccal, rectal, pleural, peritoneal, vaginal, epidural, otic, intraocular, or intrathecal route.

40-41. (canceled)

42. The method of claim 39, wherein the protein comprises at least one selected from the group consisting of an antibody, a hormone, immunomodulator, cytokine, interferon, interleukin, and enzyme.

43-53. (canceled)

54. The method of claim 39, wherein the disease or disorder is selected from the group consisting of colon cancer, rectum cancer, lung cancer, glioblastoma, renal cell cancer, non-small cell lung cancer, small cell lung cancer, asthma, respiratory syncytial virus (RSV) infection, and any combinations thereof.

55-61. (canceled)

62. A kit comprising a composition comprising at least one protein nanoparticle, the kit further comprising an applicator; and an instructional material for the use of the kit, wherein the instruction material comprises instructions for treating, ameliorating or preventing a disease or disorder in a subject in need thereof,
wherein the protein nanoparticle is prepared by a method comprising adjusting the pH of a solution comprising a protein and a non-ionic surfactant to about the isoelectric point of the protein, wherein the concentration of the non-ionic surfactant in the solution ranges from about 5% to about 20,000% of the CMC of the surfactant, thereby forming a precipitate comprising the protein nanoparticle, wherein the protein nanoparticle comprises at least a fraction of the protein and at least a fraction of the non-ionic surfactant.

63. The kit of claim 62, wherein the nanoparticle further comprises at least one selected from the group consisting of at least one therapeutic agent and at least one cell surface receptor ligand.

64-65. (canceled)


SEQUENCE LISTING (2 sequences)

SEQ ID NO: 1 - DNA, 21 bp, Artificial Sequence
  Chemically Synthesized
guuggagcuaguggcguagnn

SEQ ID NO: 2 - DNA, 21 bp, Artificial Sequence
  Chemically Synthesized
cuacgccacuagcuccaacnn